SEQUENCE LISTING (4 sequences)

SEQ ID NO: 1 - protein, 4 aa, Artificial Sequence
  Synthesized Peptide
MTRV

SEQ ID NO: 2 - protein, 4 aa, Artificial Sequence
  Synthesized Peptide
AQGV

SEQ ID NO: 3 - protein, 4 aa, Artificial Sequence
  Synthesized Peptide
LQGV

SEQ ID NO: 4 - protein, 4 aa, Artificial Sequence
  Synthesized Peptide
LAGV

(19) United States
(12) Patent Application Publication
(10) Pub. No.: US 2009/0042807 A1
(43) Pub. Date: Feb. 12, 2009

(54) Oligopeptide treatment of ischemia reperfusion injury

(76) Inventors: Nisar Ahmed Khan, Rotterdam (NL); Robbert Benner, Barendrecht (NL); Bartholomeus Caspar Jacobs, Rotterdam (NL)

Correspondence Address: TRASK BRITT, P.O. BOX 2550, SALT LAKE CITY, UT, 84110, US

(21) Appl. No.: 12/074,020

(22) Filed: Feb. 29, 2008

Related U.S. Application Data
(63) Continuation of application No. 11/706,176, filed on Feb. 12, 2007, now abandoned.
Application No. 11/706,176 is a continuation-in-part of application No. 10/821,256, filed on Apr. 8, 2004.
Application No. 10/821,256 is a continuation-in-part of application No. 10/409,642, filed on Apr. 8, 2003.
Application No. 10/409,642 is a continuation-in-part of application No. 10/028,075, filed on Dec. 21, 2001.
Application No. 11/706,176 is a continuation-in-part of application No. 10/029,206, filed on Dec. 21, 2001, now Pat. No. 7,175,679.
Application No. 10/029,206 is a continuation-in-part of application No. 09/821,380, filed on Mar. 29, 2001, now Pat. No. 6,844,315.
Application No. 09/821,380 is a continuation-in-part of application No. 09/716,777, filed on Nov. 20, 2000, now Pat. No. 6,921,751.
Application No. 09/716,777 is a continuation of application No. PCT/NL99/00313, filed on May 20, 1999.

(30) Foreign Application Priority Data
May 20, 1998 (EP) 98201695.8
Aug. 12, 1998 (EP) 98202706.2

(51) Int. Cl. A61K 38/08; A61K 38/07; A61P 9/10

(52) U.S. Cl. 514/17; 514/18

(57) ABSTRACT

The invention relates to the modulation of gene expression in a cell, also called gene control, in particular, in relation to the treatment of ischemic-reperfusion injury. The invention provides a method for modulating expression of a gene in a cell comprising providing the cell with a signaling molecule comprising a peptide or functional analogue thereof. The invention provides a method of treating ischemia-reperfusion injury in a subject by reducing NO production by the subject's macrophages, the method comprising: administering to the subject a composition comprising: means for reducing production of NO by a cell, together with a pharmaceutically acceptable excipient.

### CROSS-REFERENCE TO RELATED APPLICATIONS

This application is a continuation of U.S. patent application Ser. No. 11/706,176, filed Feb. 12, 2007, pending, which application is a continuation-in-part of co-pending U.S. patent application Ser. No. 10/821,256, filed Apr. 8, 2004 (which is a priority application of PCT/EP2005/003707), U.S. patent application Ser. No. 10/409,642, filed Apr. 8, 2003, which is a continuation-in-part of U.S. patent application Ser. No. 10/028,075, filed Dec. 21, 2001 (which is a priority application of PCT/NL02/00639), and of U.S. patent application Ser. No. 10/029,206, filed Dec. 21, 2001, now U.S. Pat. No. 7,175,679, issued Feb. 13, 2007, which is a continuation-in-part of U.S. patent application Ser. No. 09/821,380, now U.S. Pat. No. 6,844,315, filed on Mar. 29, 2001 (concomitantly with PCT/NL01/00259) which application itself was a continuation-in-part of U.S. patent application Ser. No. 09/716,777, filed Nov. 20, 2000, now U.S. Pat. No. 6,921,751, which application was a continuation of International Application PCT/NL99/00313, filed May 20, 1999, designating the United States of America, and claims priority from European Patent Office (EPO) 98201695.8, filed May 20, 1998 and EPO 98202706.2 filed Aug. 12, 1998 the contents of the entirety of all of which are incorporated herein by this reference.

### STATEMENT ACCORDING TO 37 C.F.R. § 1.52(e)(5)—SEQUENCE LISTING SUBMITTED ON COMPACT DISC

Pursuant to 37 C.F.R. § 1.52(e)(1)(ii), a compact disc containing an electronic version of the Sequence Listing has been submitted concomitant with this application, the contents of which are hereby incorporated by reference. A second compact disc is submitted and is an identical copy of the first compact disc. The discs are labeled “copy 1” and “copy 2,” respectively, and each disc contains one file entitled “Sequence Listing as submitted.txt” which is 51 KB and created on Feb. 12, 2007.

### TECHNICAL FIELD

The current invention relates to the body's innate way of modulating important physiological processes and builds upon insights reported in PCT International Publications and applications WO99/59717, WO01/00259 and PCT/NL02/00639. The invention relates generally to biotechnology and methods and means for treating disease. More particularly, the invention relates to the modulation of gene expression in a cell, also called “gene control,” in relation to the treatment of a variety of diseases such as caused by ischemia-reperfusion damage. The invention in particular relates to the treatment of acute ischemic-reperfusion injury such as seen after an ischemic event seen with stroke, myocardial infarction, extensive bleeding or embolic events, in general after the disruption of blood flow to (parts) of an organ or the organism as a whole.

### BACKGROUND

In the above identified applications, small gene-regulatory peptides are described that are derived from proteolytic breakdown of placental gonadotropins, such as human chorionic gonadotropin (hCG) produced during pregnancy. These peptide fragments (in their active state often at about 4 to 6 amino acids long) were shown to have unsurpassed immunological activity exerted by regulating expression of genes encoding inflammatory mediators such as cytokines. Surprisingly, it was found that breakdown of hCG provides a cascade of peptides that can help maintain a pregnant woman's immunological homeostasis. These peptides are substances that can balance the immune system. Where it was generally thought that the smallest breakdown products of proteins had no specific biological function on their own (except to serve as antigen for the immune system), it now emerges that the body in fact can utilize peptide fragments that control the expression of the body's own genes. Apparently, the body can use a gene-control system ruled by small fragments of the exact proteins encoded by its own genes.

It has been long known that, during pregnancy, the maternal system introduces a status of temporary immuno-modulation which results in suppression of maternal rejection responses directed against the fetus. Paradoxically, during pregnancy, often the mother's resistance to infection is increased and she is found to be better protected against the clinical symptoms of various auto-immune diseases such as rheumatism and multiple sclerosis. The protection of the fetus thus cannot be interpreted only as a result of immune suppression. Each of the above patent applications has provided insights by which the immunological balance between protection of the mother and protection of the fetus can be understood.

It was shown that certain short fragments of hCG (i.e., short peptides which can easily be synthesized, and, if need be, modified, and used as pharmaceutical composition) exert a major regulatory activity on pro- or anti-inflammatory cytokine cascades governed by a family of crucial transcription factors, the NF-κB family which stands central in regulating the expression of genes that shape the body's immune response.

Most of the hCG produced during pregnancy is produced by cells of the placenta, the organ where cells and tissues of mother and child most intensely meet and where immuno-modulation is most needed to fight off rejection. Being produced locally, the gene-regulatory peptides that are broken down from hCG in the placenta immediately balance the pro- or anti-inflammatory cytokine cascades found in the no-mans land between mother and child. Being produced by the typical placental cell, the trophoblast, the peptides traverse extracellular space; enter cells of the immune system, and exert their immuno-modulatory activity by modulating NF-κB-mediated expression of cytokine genes, thereby keeping the immunological response(s) in the placenta at bay.

Consequently, a novel therapeutic inroad is provided, using the pharmaceutical potential of gene-regulatory peptides and derivatives thereof. Indeed, evidence of specific up- or down-regulation of NF-κB driven pro- or anti-inflammatory cytokine cascades that are each, and in concert, directing the body's immune response was found in silico in gene-arrays by expression profiling studies, in vitro after treatment of immune cells and in vivo in experimental animals treated with gene-regulatory peptides. Also, considering that NF-κB is a primary effector of disease (A. S. Baldwin, J. Clin. Invest., 2001, 107:3-6), using the hCG derived gene-regulatory peptides offer significant potential for the treatment of a variety of human and animal diseases, thereby tapping the pharmaceutical potential of the exact substances that help balance the mother's immune system such that her pregnancy is safely maintained.

Gene control is generally thought to occur at four levels: 1) transcription (either initiation or termination), 2) processing of primary transcripts, 3) stabilization or destabilization of mRNAs, and 4) mRNA translation. The primary function of gene control in cells is to adjust the enzymatic machinery of the cell to its nutritional, chemical and physical environment.

Gene expression is generally thought to be regulated at both the level of transcription and translation. Modulation or regulation of gene expression requires factors called transcriptional factors. The term “gene control or regulation” also refers to the formation and use of mRNA. Although control can be exerted at a number of different molecular steps, differential transcription probably most frequently underlies the differential rate of protein synthesis in prokaryotes as well as eukaryotes. It is generally thought that activator proteins (also called transcription factors or transcriptional activators) bind to DNA and recruit the transcriptional machinery in a cell to a promoter, thereby stimulating gene expression. Further, differential processing of RNA transcripts in the cell nucleus, differential stabilization of mRNA in the cytoplasm, and differential translation of mRNA into protein are also important in eukaryotic gene control. These steps define the regulatory decisions in a transcriptional circuit and misregulation at any stage can result in a variety of diseases.

Where, in unicellular organisms, be they of prokaryotic or eukaryotic origin, a cell's response to its environment is influenced by many stimuli from the outside world, reflecting the often widely variable environment of the single cell, most cells in multicellular organisms experience a fairly constant environment. Perhaps for this reason, genes that are devoted to responses to environmental changes constitute a much smaller fraction of the total number of genes in multicellular organisms than in single-cell organisms.

As stated above, cells react to environmental changes, which they perceive through extracellular signals. These signals can be either physical (e.g., light, temperature, pressure and electricity) or chemical (e.g., food, hormones and neurotransmitters). Cells can both sense and produce signals. This makes it possible that they communicate with each other. In order to achieve this, there are complex signal-sensing and signal-producing mechanisms in unicellular and multicellular organisms.

Two groups of chemical signals can be distinguished: membrane-permeable and membrane-impermeable signals. The membrane-permeable signal molecules comprise the large family of steroid hormones, such as estrogens, progesterone, and androgens. Steroids pass the plasma membrane and bind to specific receptors, which are localized in the cytoplasm or nucleus of the cell. After binding of the hormone, the receptor undergoes a conformational change. The receptor is then able to bind to DNA itself or to proteins which can in turn interact with DNA. In general, steroid hormones can directly regulate gene expression by means of this process. The membrane-impermeable signal molecules include acetylcholine, growth factors, extracellular matrix components, thrombin, lysophosphatidic acid, the yeast mating factors and, for the social amoeba Dictyostellium discoideum, folic acid, and cyclic AMP. They are recognized by receptors, which are localized on the plasma membrane of the cell. The receptors are specific for one particular signal molecule or a family of closely related signal molecules. Upon binding of their ligands, these receptors transduce the signals by several mechanisms.

The most characteristic and exacting requirement of gene control on multicellular organisms is the execution of precise developmental decisions so that the right gene is activated in the right cell at the right time. These developmental decisions include not only those related to the development of an organism per se, as, e.g., can be seen during embryogenesis and organogenesis, or in response to disease but also relate to the differentiation or proliferation or apoptosis of those cells that merely carry out their genetic program essentially without leaving progeny behind.

Such cells, such as skin cells, precursors of red blood cells, lens cells of the eye, and antibody-producing cells, are also often regulated by patterns of gene control that serve the need of the whole organism and not the survival of an individual cell.

It is generally reasoned that there are at least three components of gene control: molecular signals, levels and mechanisms. First, it is reasoned that specific signaling molecules exist to which a specific gene can respond. Second, control is exerted on one or more levels (i.e., the step or steps) in the chain of events leading from the transcription of DNA to the use of mRNA in protein synthesis. Third, at each of those levels, specific molecular mechanisms are employed to finally exert the control over the gene to be expressed.

Many genes are regulated not by a signaling molecule that enters the cells but by molecules that bind to specific receptors on the surface of cells. Interaction between cell-surface receptors and their ligands can be followed by a cascade of intracellular events including variations in the intracellular levels of so-called second messengers (diacylglycerol, Ca²⁺, cyclic nucleotides). The second messengers in turn lead to changes in protein phosphorylation through the action of cyclic AMP, cyclic GMP, calcium-activated protein kinases, or protein kinase C, which is activated by diaglycerol.

Many of the responses to binding of ligands to cell-surface receptors are cytoplasmatic and do not involve immediate gene activation in the nucleus. Some receptor-ligand interactions, however, are known to cause prompt nuclear transcriptional activation of a specific and limited set of genes. For example, one proto-oncogene, c-fos, is known to be activated in some cell types by elevation of almost every one of the known second messengers and also by at least two growth factors, platelet-derived growth factor and epidermal growth factor. However, progress has been slow in determining exactly how such activation is achieved. In a few cases, the transcriptional proteins that respond to cell-surface signals have been characterized.

One of the clearest examples of activation of a pre-existing inactive transcription factor following a cell-surface interaction is the nuclear factor (NF)-κB, which was originally detected because it stimulates the transcription of genes encoding immunoglobulins of the kappa (κ) class in B-lymphocytes. The binding site for NF-κB in the kappa gene is well defined (see, e.g., P. A. Baeuerle and D. Baltimore, 1988, Science 242:540), providing an assay for the presence of the active factor. This factor exists in the cytoplasm of lymphocytes complexed with an inhibitor. Treatment of the isolated complex in vitro with mild denaturing conditions dissociates the complex, thus freeing NF-κB to bind to its DNA site. Release of active NF-κB in cells is now known to occur after a variety of stimuli including treating cells with bacterial lipopolysaccharide (LPS) and extracellular polypeptides as well as chemical molecules (e.g., phobol esters) that stimulate intracellular phosphokinases. Thus, a phosphorylation event triggered by many possible stimuli may account for NF-κB conversion to the active state. The active factor is then translocated to the cell nucleus to stimulate transcription only of genes with a binding site for active NF-κB.

The inflammatory response involves the sequential release of mediators and the recruitment of circulating leukocytes that become activated at the inflammatory site and release further mediators (Nat. Med. 7:1294; 2001). This response is self-limiting and resolves through the release of endogenous anti-inflammatory mediators and the clearance of inflammatory cells.

Considering that NF-κB is thought by many to be a primary effector of disease (A. S. Baldwin, J. Clin. Invest., 2001, 107:3-6), numerous efforts are underway to develop safe inhibitors of NF-κB to be used in treatment of both chronic and acute disease situations. Specific inhibitors of NF-κB should reduce side effects associated with drugs such as NSAIDS and glucocorticoids and would offer significant potential for the treatment of a variety of human and animal diseases. Specific diseases or syndromes where patients would benefit from NF-κB inhibition vary widely, and range from rheumatoid arthritis, atherosclerosis, multiple sclerosis, chronic inflammatory demyelinating polyradiculoneuritis, asthma, inflammatory bowel disease, to Helicobacter pylori-associated gastritis and other inflammatory responses, and a variety of drugs that have effects on NF-κB activity, such as corticosteroids, sulfasalazine, 5-aminosalicylic acid, aspirin, tepoxalin, leflunomide, curcumin, antioxidants and proteasome inhibitors. These drugs are considered to be non-specific and often only applicable in high concentrations that may end up being toxic for the individual treated.

Inactive cytoplasmatic forms of transcription factors can thus be activated by removal of an inhibitor, as is the case with NF-κB or, alternatively, by association of two (or more) proteins, neither of which is active by itself as in the case of interferon-α-stimulated factor (D. E. Levy et al., 1989, Genes and Development 3:1362). After interferon-α attaches to its cell-surface receptor, one of the proteins is changed within a minute or less, and the two can combine. The active (combined) factor is then translocated to the cell nucleus to stimulate transcription only of genes with a binding site for the protein. Considering that interferon-α is a mediator of responses of the body directed at pathogens and self-antigens, modulating regulation of genes that are under influence of the interferon-α-stimulated factor would contribute to the treatment of a variety of human and animal diseases.

Other typical examples of signaling molecules that affect gene expression via cell-surface receptor interaction are polypeptide hormones such as insulin, glucagon, various growth factors such as EGF, VEGF, and so on.

The steroid hormones and their receptors represent one of the best understood cases that affect transcription. Because steroid hormones are soluble in lipid membranes, they can diffuse into cells. They affect transcription by binding to specific intracellular receptors that are site-specific DNA-binding molecules. Other examples of signaling molecules that enter the cell and act intra-cellularly are thyroid hormone (T₃), vitamin D and retinoic acid, other small lipid-soluble signaling molecules that enter cells and modulate gene expression. The characteristic DNA-binding sites for the receptors for these signaling molecules are also known as response elements.

Another example of a small molecule that is involved in regulation of gene expression is ethylene, a gas that, e.g., induces the expression of genes involved in fruit ripening. Also, small plant hormones, known as auxins and cytokinins, regulate plant growth and differentiation directly by regulating gene expression.

Given the critical role of regulatory factors in gene regulation, the development of artificial or synthetic counterparts that could be used in methods to rectify errors in gene expression has been a long-standing goal at the interface of chemistry and biology.

### DISCLOSURE OF THE INVENTION

In certain embodiments, the invention provides a method for modulating gene expression in a cell, the method comprising providing the cell with a signaling molecule comprising an oligopeptide, functional analogue, or derivative thereof. Such a molecule is herein also called “NMPF” and may be referenced by number. Since peptides, and functional analogues and derivatives of relatively short amino acid sequences, are easily synthesized these days, the invention provides a method to modulate gene expression with easily obtainable synthetic compounds such as synthetic peptides or functional analogues or derivatives thereof.

In certain embodiments, also provided is a method for the treating an inflammatory condition seen after ischemic-reperfusion injury, the method comprising administering to a subject determined or believed to be in need of such treatment a molecule comprising an oligopeptide, functional analogue, or derivative thereof, the molecule able to reduce production of NO by a cell, in particular wherein the molecule additionally is able to modulate translocation and/or activity of a gene transcription factor present in a cell, especially wherein the gene transcription factor comprises an NF-κB/Rel protein. Advantageously, in certain embodiments, provided is a method wherein the modulating translocation and/or activity of a gene transcription factor that allows modulation of TNF-α production by the cell, in particular wherein the TNF-α production is reduced.

Considering that TNF-α production is central to almost all, if not all, inflammatory conditions, reducing TNF-α production can greatly alleviate, or mitigate, a great host of inflammatory conditions that are described herein. In particular, the invention provides a method wherein the inflammatory condition comprises an acute inflammatory condition, especially when considering that with reperfusion injury, both NO and TNF-α reduction will greatly mitigate the course of disease. Table 6 lists oligopeptides according to the invention that have such modulatory effect. In particular, provided is a method of treatment wherein the treatment comprises administering to the subject a pharmaceutical composition comprising an oligopeptide or functional analogue or derivative thereof capable of reducing production of NO by a cell, preferably wherein the composition comprises at least two oligopeptides or functional analogues or derivatives thereof capable of reducing production of NO by a cell; examples of such combinations can be selected under guidance of Table 6, whereby it suffices to select two, or more, with a desired effect, such as wherein the at least two oligopeptides are selected from the group of SEQ ID NO:1, SEQ ID NO:2, and SEQ ID NO:3. Table 6 describes various “means for modulating translocation and/or activity of a gene transcription factor present in a cell.”

Also provided is an isolated, preferably synthetic, oligopeptide or functional analogue or derivative thereof or mixture of such oligopeptides or analogues or derivatives capable of reducing production of NO by a cell. Such cell is preferably of a macrophage or DC lineage, considering the central role these cells play in the inflammatory process. Also provided is a pharmaceutical composition comprising an oligopeptide or functional analogue or derivative according to the invention or comprising at least two oligopeptides or functional analogues or derivatives thereof capable of reducing production of NO by a cell. Furthermore, provided is the use of an oligopeptide or functional analogue or derivative thereof able to reduce NO production by a cell for producing a pharmaceutical composition for the treatment of an inflammatory condition by the reduction of NO production by macrophages or DC in the subject to be treated.

A functional analogue or derivative of a peptide is an amino acid sequence, or other sequence monomers, which has been altered such that the functional properties of the sequence are essentially the same in kind, but not necessarily in amount. An analogue or derivative can be provided in many ways, for instance, through conservative amino acid substitution. Also peptidomimetic compounds can be designed that functionally or structurally resemble the original peptide taken as a starting point but that are, e.g., composed of non-naturally occurring amino acids or polyamides. With conservative amino acid substitution, one amino acid residue is substituted with another residue with generally similar properties (size, hydrophobicity, etc.), such that the overall functioning is likely not to be seriously affected. However, it is often much more desirable to improve a specific function. A derivative can also be provided by systematically improving at least one desired property of an amino acid sequence. This can, for instance, be done by an Ala-scan and/or replacement net mapping method. With these methods, many different peptides are generated, based on an original amino acid sequence but each containing a substitution of at least one amino acid residue. The amino acid residue may either be replaced by alanine (Ala-scan) or by any other amino acid residue (replacement net mapping). This way, many positional variants of the original amino acid sequence are synthesized. Every positional variant is screened for a specific activity. The generated data are used to design improved peptide derivatives of a certain amino acid sequence.

A derivative or analogue can also, for instance, be generated by substitution of an L-amino acid residue with a D-amino acid residue. This substitution, leading to a peptide which does not naturally occur in nature, can improve a property of an amino acid sequence. It is, e.g., useful to provide a peptide sequence of known activity of all D-amino acids in retro inversion format, thereby allowing for retained activity and increased half-life values. By generating many positional variants of an original amino acid sequence and screening for a specific activity, improved peptide derivatives comprising such D-amino acids can be designed with further improved characteristics.

A person skilled in the art is well able to generate analogous compounds of an amino acid sequence. This can, for instance, be done through screening of a peptide library. Such an analogue has essentially the same functional properties of the sequence in kind, but not necessarily in amount. Also, peptides or analogues can be circularized (for example, by providing them with (terminal) cysteines, dimerized or multimerized, for instance, by linkage to lysine or cysteine or other compounds with side-chains that allow linkage or multimerization, brought in tandem or repeat configuration, conjugated or otherwise linked to carriers known in the art, if only by a labile link that allows dissociation.

In certain embodiments, the invention also provides a signaling molecule for modulating expression of a gene in a cell comprising a small peptide or functional analogue or derivative thereof. Surprisingly, a small peptide acts as a signaling molecule that can modulate signal transduction pathways and gene expression. A functional analogue or derivative of a small peptide that acts as such a signaling molecule for modulating expression of one or more genes in a cell can be identified or obtained by at least one of various methods for finding such a signaling molecule as provided herein.

For example, one method as provided herein for identifying or obtaining a signaling molecule comprising a peptide or functional derivative or analogue thereof capable of modulating expression of a gene in a cell comprises providing the cell with a peptide or derivative or analogue thereof and determining the activity and/or nuclear translocation of one or more gene transcription factors. Such activity can be determined in various ways using means and/or methods honed to the specific transcription factor(s) under study. In the detailed description, it is provided to study NF-κB/Rel protein translocation and/or activity, but it is, of course, also easily possible to study translocation and/or activity of any other transcription factor for which such tools are available or can be designed. One such other transcription factor is, e.g., the interferon-α-stimulated factor as discussed above. Other useful transcription factors to study in this context comprise c-Jun, ATF-2, Fos, and their complexes, ELK-1, EGR-1, IRF-1, IRF-3/7, AP-1, NF-AT, C/EBPs, Sp1, CREB, PPARγ, and STAT proteins to name a few. Considering that many proteins are subject to proteolytic breakdown whereby oligopeptide fragments are generated, many already before the full protein even has exerted a function, it is hereby established that oligopeptide fragments of such proteins (of which a non-extensive list is given in the detailed description, but one can, e.g., think of MAPKK-2 that can give rise to a peptide MLARRKPVLPALTINP (SEQ ID NO:4), and subsequently to a peptide comprising MLARRKP (SEQ ID NO:5) or MLAR (SEQ ID NO:6) or VLPALT (SEQ ID NO:7) or VLPAL (SEQ ID NO:8), but also of nitric oxide synthase that can give rise to peptides FPGC (SEQ ID NO:9) or PGCP (SEQ ID NO:10), GVLPAVP (SEQ ID NO:11), LPA, VLPAVP (SEQ ID NO:12), or PAVP (SEQ ID NO:13) after proper proteolysis) are involved in feedback mechanisms regulating gene expression, likely by the effect of transcription factors on gene expression. In addition, oligopeptide fragments of proteins (of which a non-extensive list is given in the detailed description) can also modulate the activity of extracellular components such as factor XIII (examples of oligopeptide fragments obtained from factor XIII are LQGV (SEQ ID NO:1), LQGVVPRGV (SEQ ID NO:14), GVVP (SEQ ID NO:15), VPRGV (SEQ ID NO:16), PRG, PRGV (SEQ ID NO:17)) or activated protein C (APC) and thereby eventually lead to the modulation of intracellular signal transduction pathways and gene(s) expression.

As said, the invention provides active oligopeptides acting as a signaling molecule. To allow for improved bio-availability of such a signaling molecule (which is useful as a pharmacon, especially when produced artificially), the invention also provides a method for determining whether a small peptide or derivative or analogue thereof can act as a functional signaling molecule according to the invention, the method further comprising determining whether the signaling molecule is membrane-permeable.

In certain embodiments, the invention provides a method for obtaining information about the capacity or tendency of an oligopeptide, or a modification or derivative thereof, to regulate expression of a gene comprising: contacting the oligopeptide, or a modification or derivative thereof, with at least one cell; determining the presence of at least one gene product in or derived from the cell. It is preferred that the oligopeptide comprises an amino acid sequence corresponding to a fragment of a naturally occurring polypeptide, such as hCG, or MAPKK (SEQ ID NO:18), or another kinase, be it of plant or animal cell, or of eukaryotic or prokaryotic origin, or a synthase of a regulatory protein in a cell, such as wherein the regulatory protein is a (pro-) inflammatory mediator, such as a cytokine. Several candidate proteins and peptide fragments are listed in the detailed description which are a first choice for such an analysis from the inventors' perspective, but the person skilled in the art, and working in a specific field of interest in biotechnology, shall immediately understand which protein to select for such analyses for his or her own purposes related to his or her field.

In particular, it is provided to perform a process according to the invention further comprising a further step comprising determining the presence of the gene product in or derived from a cell which has not been contacted with the oligopeptide, or a modification or derivative thereof, and determining the ratio of gene product found in step b to gene product found in step c, as can easily be done with the present-day genechip technology (see, e.g., the detailed description herein) and related methods of expression profiling known in the art.

Another method provided herein for identifying or obtaining information on a signaling molecule, (or for that matter, the signaling molecule itself, considering that the next step of synthesizing the molecule, generally being a short peptide, is whole within the art) comprising a peptide or functional derivative or analogue thereof capable of modulating expression of a gene in a cell comprises providing the cell with a peptide or derivative or analogue thereof and determining relative up-regulation and/or down-regulation of at least one gene expressed in the cell. The up-regulation can classically be studied by determining via, e.g., Northern or Western blotting or nucleic acid detection by PCR or immunological detection of proteins whether a cell or cells make more (in the case of up-regulation) or less (in the case of down-regulation) of a gene expression product such as mRNA or protein after the cell or cells have been provided with the peptide or derivative or analogue thereof. Of course, various methods of the invention can be combined to better analyze the functional analogue of the peptide or derivative or analogue under study. Furthermore, relative up-regulation and/or down-regulation of a multitude or clusters of genes expressed in the cell can be easily studied as well, using libraries of positionally or spatially addressable predetermined or known relevant nucleic acid sequences or unique fragments thereof bound to an array or brought in an array format, using, e.g., a nucleic acid library or so-called gene-chip expression analysis systems. Lysates of cells or preparations of cytoplasma and/or nuclei of cells that have been provided with the peptide or derivative or analogue under study are than contacted with the library, and relative binding of, e.g., mRNA to individual nucleic acids of the library is then determined, as further described herein in the detailed description.

A functional analogue or derivative of a small peptide that can act as a signaling molecule for modulating expression of a gene in a cell can also be identified or obtained by a method for identifying or obtaining a signaling molecule comprising an oligopeptide or functional derivative or analogue thereof capable of modulating expression of a gene in a cell comprising providing a peptide or derivative or analogue thereof and determining binding of the peptide or derivative or analogue thereof to a factor related to gene control. Such a factor related to gene control can be any factor related to transcription (either initiation or termination), processing of primary transcripts, stabilization or destabilization of mRNAs, and mRNA translation.

Binding of a peptide or derivative or analogue thereof to such a factor can be determined by various methods known in the art. Classically, peptides or derivatives or analogues can be (radioactively) labeled and binding to the factor can be determined by detection of a labeled peptide-factor complex, such as by electrophoresis, or other separation methods known in the art. However, for determining binding to such factors, array techniques, such as used with peptide libraries, can also be employed, comprising providing a multitude of peptides or derivatives or analogues thereof and determining binding of at least one of the peptides or derivatives or analogues thereof to a factor related to gene control.

In a preferred embodiment, the factor related to gene control comprises a transcription factor, such as an NF-κB-Rel protein or another transcription factor desired to be studied. When binding of a functional analogue according to the invention to such factor has been established, it is of course possible to further analyze the analogue by providing a cell with the peptide or derivative or analogue thereof and determining the activity and/or nuclear translocation of a gene transcription factor in the cell, and/or by providing a cell with the peptide or derivative or analogue thereof and determining relative up-regulation and/or down-regulation of at least one gene expressed in the cell.

The invention thus provides a signaling molecule useful in modulating expression of a gene in a cell and/or useful for reducing NO production by a cell and identifiable or obtainable by employing a method according to the invention. Useful examples of such a signaling molecule can be selected from the group of oligopeptides LQG, AQG, LQGV (SEQ ID NO:1), AQGV (SEQ ID NO:2), LQGA (SEQ ID NO:19), VLPALPQVVC (SEQ ID NO:20), VLPALP (SEQ ID NO:3), ALPALP (SEQ ID NO:21), VAPALP (SEQ ID NO:22), ALPALPQ (SEQ ID NO:23), VLPAAPQ (SEQ ID NO:24), VLPALAQ (SEQ ID NO:25), LAGV (SEQ ID NO:26), VLAALP (SEQ ID NO:27), VLPALA (SEQ ID NO:28), VLPALPQ (SEQ ID NO:29), VLAALPQ (SEQ ID NO:30), VLPALPA (SEQ ID NO:31), GVLPALP (SEQ ID NO:32), GVLPALPQ (SEQ ID NO:33), LQGVLPALPQVVC (SEQ ID NO:34), VVCNYRDVRFESIRLPGCPRGVNPVVSYAVALSC QCAL (SEQ ID NO:35), RPRCRPINATLAVEK (SEQ ID NO:36), EGCPVCITVNTTICAGYCPT (SEQ ID NO:37), SKAPPPSLPSPSRLPGPS (SEQ ID NO:38), SIRLPGCPRGVNPVVS (SEQ ID NO:39), LPGCPRGVNPVVS (SEQ ID NO:40), LPGC (SEQ ID NO:41), MTRV (SEQ ID NO:42), MTR, VVC, QVVC (SEQ ID NO:43) and functional analogues or derivatives thereof.

A preferred size of a signaling molecule according to the invention is at most 30 to 40 amino acids, although much smaller molecules, in particular of oligopeptide size, have been shown to be particularly effective. Surprisingly, the invention provides here the insight that gene expression can be modulated or regulated by small peptides that are most likely breakdown products of larger polypeptides such as chorionic gonadotrophin (CG) and growth hormones or growth factors such as fibroblast growth factor, EGF, VEGF, RNA 3′ terminal phosphate cyclase and CAP18. In principle, such regulating peptide sequences can be derived from a part of any protein of polypeptide molecule produced by prokaryotic and/or eukaryotic cells, and the invention provides the insight that breakdown products of polypeptides, preferably oligopeptides at about the sizes as provided herein, are naturally involved as signaling molecule in modulation of gene expression. In particular, as signaling molecule, a (synthetic) peptide is provided obtainable or derivable from beta-human chorionic gonadotrophin (beta-HCG), preferably from nicked beta-HCG. It was thought before that breakdown products of nicked-beta hCG were involved in immuno-modulation (PCT International Patent Publication WO99/59671) or in the treatment of wasting syndrome (PCT International Patent Publication WO97/49721), but a relationship with modulation of gene expression was not forwarded in these publications. Of course, such an oligopeptide or functional equivalent or derivative thereof is likely obtainable or derivable from other proteins that are subject to breakdown or proteolysis and that are close to a gene regulatory cascade. Preferably, the peptide signaling molecule is obtained from a peptide having at least ten amino acids such as a peptide having an amino acid sequence MTRVLQGVLPALPQVVC (SEQ ID NO:44), SIRLPGCPRGVNPVVS (SEQ ID NO:39), VVCNYRDVRFESIRLPGCPRGVNPVVSYAVALSCQCAL (SEQ ID NO:35), RPRCRPINATLAVEKEGCPVCITVNTTICAGYCPT (SEQ ID NO:45), CALCRRSTTDCGGPKD HPLTC (SEQ ID NO:46), SKAPPPSLPSPSRLPGPS(SEQ ID NO:38), CRRSTTDCGGPKDHPLTC (SEQ ID NO:47), TCDDPRFQDSSSSKAPPPSLPSPSRLPGPSDTPILPQ (SEQ ID NO:48) or functional fragment (e.g., a breakdown product) or functional analogue thereof. “Functional analogue” herein relates to the signaling molecular effect or activity as, e.g., can be measured by measuring nuclear translocation of a relevant transcription factor, such as NF-κB in an NF-κB assay, or AP-1 in an AP-1 assay, or by another method as provided herein. Fragments can be somewhat (i.e., one or two amino acids) smaller or larger on one or both sides, while still providing functional activity.

Not wishing to be bound by theory, it is postulated herein that an unexpected mode of gene regulation has been uncovered. Polypeptides, such as endogenous CG, EGF, etc., but also polypeptides of pathogens, such as viral, bacterial or protozoal polypeptides, are subject to breakdown into distinct oligopeptides, e.g., by intracellular proteolysis. Distinct proteolytic enzymes are widely available in the cell, e.g., in eukaryotes in the lysosomal or proteasomal system. Some of the resulting breakdown products are oligopeptides of three to 15, preferably four to nine, most preferably four to six, amino acids long that are surprisingly not without any function or effect to the cell but, as demonstrated herein, may be involved, possibly via a feedback mechanism in the case of breakdown of endogenous polypeptides, as signaling molecules in the regulation of gene expression, as demonstrated herein by the regulation of the activity or translocation of a gene transcription factor such as NF-κB by, e.g., peptide LQGV (SEQ ID NO:1), VLPALPQVVC (SEQ ID NO:20), LQGVLPALPQ (SEQ ID NO:49), LQG, GVLPALPQ (SEQ ID NO:33), VLPALP (SEQ ID NO:3), VLPALPQ (SEQ ID NO:29), GVLPALP (SEQ ID NO:32), VVC, MTRV (SEQ ID NO:42), MTR. Synthetic versions of these oligopeptides, as described above, and functional analogues or derivatives of these breakdown products are herein provided to modulate gene expression in a cell and be used in methods to rectify errors in gene expression or the treatment of disease. Oligopeptides such as LQG, AQG, LQGV (SEQ ID NO:1), AQGV (SEQ ID NO:2), LQGA (SEQ ID NO:19), VLPALP (SEQ ID NO:3), ALPALP (SEQ ID NO:21), VAPALP (SEQ ID NO:22), ALPALPQ (SEQ ID NO:23), VLPAAPQ (SEQ ID NO:24), VLPALAQ (SEQ ID NO:25), LAGV (SEQ ID NO:26), VLAALP (SEQ ID NO:27), VLPALA (SEQ ID NO:28), VLPALPQ (SEQ ID NO:29), VLAALPQ (SEQ ID NO:30), VLPALPA (SEQ ID NO:31), GVLPALP (SEQ ID NO:32), GVLPALPQ (SEQ ID NO:33), LQGVLPALPQVVC (SEQ ID NO:34), SIRLPGCPRGVNPVVS (SEQ ID NO:39), SKAPPPSLPSPSRLPGPS (SEQ ID NO:38), LPGCPRGVNPVVS (SEQ ID NO:40), LPGC (SEQ ID NO:41), MTRV (SEQ ID NO:42), MTR, VVC, or functional analogues or derivatives (including breakdown products) of the longer sequences thereof, are particularly effective.

By using the insight as expressed herein, in a preferred embodiment, the invention provides a method for modulating expression of a gene in a cell comprising providing the cell with a signaling molecule comprising an oligopeptide or functional analogue or derivative thereof wherein the signaling molecule is membrane-permeable in that it enters the cell. Most small peptides as described herein have already an inherent propensity to become intracellularly involved, but signaling molecules as provided herein can also be provided with additional peptide sequences, such as arginine- or lysine-rich stretches of amino acids, that allow for improved internalization across a lipid bilayer membrane, and may possibly be cleaved off later by internal proteolytic activity.

In a preferred embodiment, the invention provides a method for modulating expression of a gene in a cell comprising providing the cell with a signaling molecule comprising a small peptide (amino acid sequence) or functional analogue or derivative thereof, wherein the signaling molecule modulates NF-κB/Rel protein conversion or translocation. As said, NF-κB was originally identified as a gene transcription factor that bound to an enhancer element in the gene for the Igκ light chain and was believed to be B-cell-specific. However, subsequent studies revealed that NF-κB/Rel proteins are ubiquitously expressed and play a central role as transcription factor in regulating the expression of many genes, particularly those involved in immune, inflammatory, developmental and apoptotic processes. NF-κB-related gene transcription factors can be activated by different stimuli such as microbial products, proinflammatory cytokines, T- and B-cell mitogens, and physical and chemical stresses. NF-κB in turn regulates the inducible expression of many cytokines, chemokines, adhesion molecules, acute phase proteins, and antimicrobial peptides.

NF-κB represents a group of structurally related and evolutionarily conserved gene transcription factors. So far, five mammalian NF-κB proteins named Rel (c-Rel), RelA (p65), RelB, NF-κB1 (p50 and its precursor p105), and NF-κB2 (p52 and it precursor p100) have been described. NF-κB proteins can exist as homo- or heterodimers, and although most NF-κB dimers are activators of transcription, the p50/p50 and p52/p52 homodimers often repress the transcription of their target genes. In Drosophila, three NF-κB homologs named Dorsal, Dif, and Relish have been identified and characterized. Structurally, all NF-κB/Rel proteins share a highly conserved NH₂-terminal Rel homology domain (RHD) that is responsible for DNA binding, dimerization, and association with inhibitory proteins known as IκBs. In resting cells, NF-κB/Rel dimers are bound to IκBs and retained in an inactive form in the cytoplasm. Like NF-κB, IκBs are also members of a multigene family containing seven known mammalian members including IκBα, IκBβ, Iκbγ, IκBε, Bcl-3, the precursor Rel-proteins, p100, and p105, and one Drosophila IκB named Cactus. The IκB family is characterized by the presence of multiple copies of ankyrin repeats, which are protein-protein interaction motifs that interact with NF-κB via the RHD. Upon appropriate stimulation, IκB is phosphorylated by IκB kinases (IKKs), polyubiquitinated by a ubiquitin ligase complex, and degraded by the 26S proteosome. Consequently, NF-κB is released and translocates into the nucleus to initiate gene expression.

NF-κB-related transcription factors regulate the expression of a wide variety of genes that play critical roles in innate immune responses. Such NF-κB target genes include those encoding cytokines (e.g., IL-1, IL-2, IL-6, IL-12, TNF-α, LTα, LTβ, and GM-CSF), adhesion molecules (e.g., ICAM, VCAM, endothelial leukocyte adhesion molecule [ELAM]), acute phase proteins (e.g., SAA), and inducible enzymes (e.g., iNOS and COX-2). In addition, it has been demonstrated that several evolutionary conserved antimicrobial peptides, e.g., β-defensins, are also regulated by NF-κB, a situation similar to Drosophila. Besides regulating the expression of molecules involved in innate immunity, NF-κB also plays a role in the expression of molecules important for adaptive immunity, such as MHC proteins, and the expression of critical cytokines such as IL-2, IL-12 and IFN-γ. NF-κB plays an important role in the overall immune response by affecting the expression of genes that is critical for regulating the apoptotic process, such as c-IAP-1 and c-IAP-2, Fas ligand, c-myc, p53, and cyclin D1.

Under normal conditions, NF-κB is rapidly activated upon microbial and viral invasion, and this activation usually correlates with resistance of the host to infection. However, persistent activation of NF-κB may lead to the production of excessive amounts of pro-inflammatory mediators such as IL-12 and TNF-α, resulting in tissue damage, as in insulin-dependent diabetes mellitus, atherosclerosis, Crohn's disease, organ failure, and even death of the host, as in bacterial infection-induced septic shock. It is interesting to note that in order to survive in the host, certain pathogens, such as Bordetella, Yersinia, Toxoplasma gondii and African Swine Fever Virus, have evolved mechanisms to counteract or escape the host system by inhibiting NF-κB activation. On the other hand, some viruses, including HIV-1, CMV and SV-40, take advantage of NF-κB as a host factor that is activated at sites of infection.

Furthermore, in certain embodiments, the invention provides a method to explore alterations in gene expression in antigen-presenting cells such as dendritic cells in response to microbial exposure by analyzing a gene-expression profile of dendritic cells in response to microorganisms such as, e.g., bacteria such as E. coli, or other pathogenic bacteria, fungi or yeasts such as C. albicans, viruses such as influenza virus and the effect of (simultaneous) treatment of these diseases with a signaling molecule according to the invention. For example, human monocyte-derived dendritic cells are cultured with one or more pathogens for one to 36 hours, and gene expression is analyzed using an oligonucleotide array representing a (be it large or small) set of genes. When the pathogens regulate the expression of a core set of a distinct number of genes, these genes may be classified according to their kinetics of expression and function. Generally, within four hours of pathogen exposure, genes associated with pathogen recognition and phagocytosis will be down-regulated, whereas genes for antigen processing and presentation are up-regulated eight hours post-exposure. Treatment of such dendritic cells with a signaling molecule according to the invention (be it simultaneous or before or after the treatment of the cells with the pathogen) allows studying the effect of a signaling molecule on the effect a pathogen has on an antigen-presenting cell.

In short, the invention surprisingly provides a signaling molecule capable of modulating expression of a gene in a cell, the molecule being a short peptide, preferably of at most 30 amino acids long or a functional analogue or derivative thereof. In a much preferred embodiment, the peptide is an oligopeptide of from about three to about 15 amino acids long, preferably four to twelve, more preferably four to nine, most preferably four to six, amino acids long, or a functional analogue or derivative thereof. Of course, such signaling molecule can be longer, e.g., by extending it (N- and/or C-terminally, with more amino acids or other side groups, which can, e.g., be (enzymatically) cleaved off when the molecule enters the place of final destination. Such extension may even be preferable to prevent the signaling molecule from becoming active in an untimely fashion; however, the core or active fragment of the molecule comprises the aforementioned oligopeptide or analogue or derivative thereof.

In particular, the invention provides a modulator of NF-κB/Rel protein activation comprising a signaling molecule according to the invention. Such modulators are widely searched after these days. Furthermore, the invention provides use of a signaling molecule according to the invention for the production of a pharmaceutical composition for the modulation of gene expression.

Also, the invention provides a method for the treatment of bone disease such as osteoporosis comprising administering to a subject in need of such treatment a molecule comprising an oligopeptide peptide or functional analogue thereof, the molecule capable of modulating production of NO and/or TNF-α by a cell. Such a method of treatment is particularly useful in post-menopausal women that no longer experience the benefits of being provided with a natural source of several of the signaling molecules as provided herein, hCG and its breakdown products. Furthermore, the invention provides a method for the treatment of an inflammatory condition associated with TNF-α activity of fibroblasts, such as seen with chronic arthritis or synovitis, comprising administering to a subject in need of such treatment a molecule comprising an oligopeptide peptide or functional analogue thereof wherein the molecule is capable of modulating translocation and/or activity of a gene transcription factor present in a cell, in particular of the NF-κB factor. Such a treatment can be achieved by systemic administration of a signaling molecule according to the invention, but local administration in joints, bursae or tendon sheaths is provided as well. The molecule can be selected from Table 6 or identified in a method according to the invention. It is preferred when the treatment comprises administering to the subject a pharmaceutical composition comprising an oligopeptide or functional analogue thereof also capable of reducing production of NO by a cell, e.g., wherein the composition comprises at least two oligopeptides or functional analogues thereof, each capable of reducing production of NO and/or TNF-α by a cell, in particular wherein the at least two oligopeptides are selected from the group SEQ ID NO:1, SEQ ID NO:2, and SEQ ID NO:3.

Furthermore, the invention provides use of an oligopeptide or functional analogue thereof capable of reducing production of NO and/or TNF-α by a cell for the production of a pharmaceutical composition for the treatment of an inflammatory condition or a post-menopausal condition, or a bone disease such as osteoporosis, or for the induction of weight loss. The term “pharmaceutical composition” as used herein is intended to cover both the active signaling molecule alone or a composition containing the signaling molecule together with a pharmaceutically acceptable carrier, diluent or excipient. Acceptable carrier, diluent or excipient of an oligopeptide as described herein include, e.g., physiological salt solutions or PBS solutions.

In one embodiment of the invention, a signal molecule is administered in an effective concentration to an animal or human systemically, for instance, by intravenous, intramuscular or intraperitoneal administration. Another way of administration comprises perfusion of organs or tissue, be it in vivo or ex vivo, with a perfusion fluid comprising a signal molecule according to the invention. Topical administration, e.g., in ointments or sprays, may also apply, e.g., in inflammations of the skin or mucosal surfaces of, e.g., mouth, nose and/or genitals. Local administration can occur in joints, bursae, tendon sheaths, in or around the spinal cord at locations where nerve bundles branch off, at the location of hernias, in or around infarcted areas in brain or heart, etc. The administration may be done as a single dose, as a discontinuous sequence of various doses, or continuously for a period of time sufficient to permit substantial modulation of gene expression. In the case of a continuous administration, the duration of the administration may vary depending upon a number of factors that would readily be appreciated by those skilled in the art.

The administration dose of the active molecule may be varied over a fairly broad range. The concentrations of an active molecule that can be administered would be limited by efficacy at the lower end and the solubility of the compound at the upper end. The optimal dose or doses for a particular patient should and can be determined by the physician or medical specialist involved, taking into consideration well-known relevant factors such as the condition, weight and age of the patient, etc.

The active molecule may be administered directly in a suitable vehicle, such as, e.g., PBS, or as solutions in alcohol or DMSO. Pursuant to preferred embodiments of the invention, however, the active molecule is administered through a single-dose delivery using a drug-delivery system, such as a sustained-release delivery system, which enables the maintenance of the required concentrations of the active molecule for a period of time sufficient for adequate modulation of gene expression. A suitable drug-delivery system would be pharmacologically inactive or at least tolerable. It should preferably not be immunogenic nor cause inflammatory reactions, and should permit release of the active molecule so as to maintain effective levels thereof over the desired time period. A large variety of alternatives is known as suitable for purposes of sustained release and is contemplated as within the scope of the invention. Suitable delivery vehicles include, but are not limited to, the following microcapsules or microspheres; liposomes and other lipid-based release systems; viscous instillates; absorbable and/or biodegradable mechanical barriers and implants; and polymeric delivery materials, such as polyethylene oxide/polypropylene oxide block copolymers, polyesters, cross-linked polyvinylalcohols, polyanhydrides, polymethacrylate and polymethacrylamide hydrogels, anionic carbohydrate polymers, etc. Useful delivery systems are well known in the art.

A highly suitable formulation to achieve the active molecule release comprises injectable microcapsules or microspheres made from a biodegradable polymer, such as poly (dl-lactide), poly (dl-lactide-co-glycolide), polycaprolactone, polyglycolide, polylactic acid-co-glycolide, poly (hydroxybutyric acid), polyesters or polyacetals. Injectable systems comprising microcapsules or microspheres having a diameter of about 50 to about 500 micrometers offer advantages over other delivery systems. For example, they generally use less active molecules and may be administered by paramedical personnel. Moreover, such systems are inherently flexible in the design of the duration and rate of separate drug release by selection of microcapsule or microsphere size, drug loading and dosage administered. Further, they can be successfully sterilized by gamma irradiation.

The design, preparation and use of microcapsules and microspheres are well within the reach of persons skilled in the art and detailed information concerning these points is available in the literature. Biodegradable polymers (such as lactide, glycolide and caprolactone polymers) may also be used in formulations other than microcapsules and microspheres; for example, pre-made films and spray-on films of these polymers containing the active molecule would be suitable for use in accordance with the invention. Fibers or filaments comprising the active molecule are also contemplated as within the scope of the invention.

Another highly suitable formulation for a single-dose delivery of the active molecule in accordance with the invention involves liposomes. The encapsulation of an active molecule in liposomes or multilamellar vesicles is a well-known technique for targeted drug delivery and prolonged drug residence. The preparation and use of drug-loaded liposomes is well within the reach of persons skilled in the art and well documented in the literature.

Yet another suitable approach for single dose delivery of an active molecule in accordance with the invention involves the use of viscous instillates. In this technique, high molecular weight carriers are used in admixture with the active molecule, giving rise to a structure that produces a solution with high viscosity. Suitable high molecular weight carriers include, but are not limited to, the following: dextrans and cyclodextrans; hydrogels; (cross-linked) viscous materials, including (cross-linked) viscoelastics; carboxymethylcellulose; hyaluronic acid; and chondroitin sulfate. The preparation and use of drug-loaded viscous instillates is well known to persons skilled in the art.

Pursuant to yet another approach, the active molecule may be administered in combination with absorbable mechanical barriers such as oxidized, regenerated cellulose. The active molecule may be covalently or non-covalently (e.g., ionically) bound to such a barrier, or it may simply be dispersed therein. A pharmaceutical composition as provided herein is particularly useful for modulating gene expression by inhibiting NF-κB/Rel protein activation.

NF-κB/Rel proteins are a group of structurally related and evolutionarily conserved proteins (Rel). Well known are c-Rel, RelA (p65), RelB, NF-κB1 (p50 and its precursor p105), and NF-κB2 (p52 and its precursor p100). Most NF-κB dimers are activators of transcription; p50/p50 and p52/p52 homodimers repress the transcription of their target genes. All NF-κB/Rel proteins share a highly conserved NH₂-terminal Rel homology domain (RHD). RHD is responsible for DNA binding, dimerization, and association with inhibitory proteins known as IκBs. In resting cells, NF-κB/Rel dimers are bound to IκBs and retained in an inactive form in the cytoplasm. IκBs are members of a multigene family (IκBα, IκBβ, IκBγ, IκBε, Bcl-3, the precursor Rel-proteins, p100 and p105). The presence of multiple copies of ankyrin repeats to interact with NF-κB via the RHD (protein-protein interaction). Upon appropriate stimulation, IκB is phosphorylated by IκB Kinase (IKKs), polyubiquitinated by ubiquitin ligase complex, and degraded by the 26S proteosome. NF-κB is released and translocates into the nucleus to initiate gene expression.

NF-κB regulation of gene expression includes innate immune responses: such as regulated by cytokines IL-1, IL-2, IL-6, IL-12, TNF-α, LTα, LT-β, GM-CSF; expression of adhesion molecules (ICAM, VCAM, endothelial leukocyte adhesion molecule [ELAM]), acute phase proteins (SAA), inducible enzymes (iNOS and COX-2) and antimicrobial peptides (beta-defensins). For adaptive immunity, MHC proteins, IL-2, IL-12 and IFN-α are regulated by NF-κB. Regulation of overall immune response includes the regulation of genes critical for regulation of apoptosis (c-IAP-1 and c-IAP-2, Fas Ligand, c-myc, p53 and cyclin D1).

Considering that NF-κB and related transcription factors are cardinal pro-inflammatory transcription factors, and considering that the invention provides a signaling molecule, such as an oligopeptide and functional analogues or derivatives thereof that are capable of inhibiting NF-κB and likely also other pro-inflammatory transcription factors, herein also called NF-κB inhibitors, the invention provides a method for modulating NF-κB activated gene expression, in particular for inhibiting the expression and thus inhibiting a central pro-inflammatory pathway.

The consequence of this potency to inhibit this pro-inflammatory pathway is wide and far-reaching. In certain embodiments, the invention provides a method to mitigate or treat inflammatory airway disease such as asthma. Generally, asthmatic patients show persistent activation of NF-κB of cells lining the respiratory tract. Providing these patients, e.g., by aerosol application, with a signaling molecule according to the invention, such as SEQ ID NO:1, SEQ ID NO:2, or SEQ ID NO:3 or a functional analogue or derivative thereof, alleviates the inflammatory airway response of these individuals by inhibiting NF-κB activation of the cells. Such compositions can advantageously be made with signaling molecules that are taken up in liposomes.

As said, inflammation involves the sequential activation of signaling pathways leading to the production of both pro- and anti-inflammatory mediators. Considering that much attention has focused on pro-inflammatory pathways that initiate inflammation, relatively little is known about the mechanisms that switch off inflammation and resolve the inflammatory response. The transcription factor NF-κB is thought to have a central role in the induction of pro-inflammatory gene expression and has attracted interest as a new target for the treatment of inflammatory disease. However, NF-κB activation of leukocytes recruited during the onset of inflammation is also associated with pro-inflammatory gene expression, whereas such activation during the resolution of inflammation is associated with the expression of anti-inflammatory genes and the induction of apoptosis. Inhibition of NF-κB during the resolution of inflammation protracts the inflammatory response and prevents apoptosis. This shows that NF-κB has an anti-inflammatory role in vivo involving the regulation of inflammatory resolution. The invention provides a tool to modulate the inflammation at the end phase; a signaling molecule or modulator as provided herein allows the modulation of the NF-κB pathway at different stages of the inflammatory response in vivo. In a particular embodiment, the invention provides a modulator of NF-κB for use in the resolution of inflammation, e.g., through the regulation of leukocyte apoptosis.

In certain embodiments, the invention also provides a method to mitigate or treat neonatal lung disease, also called chronic lung disease of prematurity, a condition often seen with premature children who develop a prolonged pulmonary inflammation or bronchopulmonary dysplasia. Treating such premature children with an NF-κB inhibitor, such as oligopeptide LQGV (SEQ ID NO:1), or functional analogue or derivative thereof, as provided herein, allows such lung conditions to be prevented or ameliorated as well.

Recent advances in bone biology provide insight into the pathogenesis of bone diseases. The invention also provides a method of treatment of a post-menopausal condition such as osteoporosis comprising modulation and inhibition of osteoclast differentiation and inhibiting TNF-α-induced apoptosis of osteoblasts, thereby limiting the dissolution of bone structures, otherwise so prominent in post-menopausal women that no longer have a natural source of hCG and thus lack the modulatory effect of the signal molecules that are derived from hCG as shown herein. The invention thus also provides a method of treatment of a bone disease, such as osteoporosis (which is often, but not exclusively seen with post-menopausal women). Furthermore, NO and TNF-α modulators as provided herein inhibit the inflammatory response and bone loss in periodontitis. Furthermore, considering that there is a correlation between TNF-α activity and severity of clinical manifestations in ankylosing spondylitis, the invention provides the treatment of spondylitis by use of a signaling molecule as provided herein.

Furthermore, considering that an important pathogenic component in the development of insulin-dependent diabetes mellitus (type 1) comprises over-activation of the NF-κB pathway as seen in dendritic cells, treatment with an NF-κB inhibitor according to the invention will lead to reduced symptoms of diabetes, or at least to a prolonged time to onset of the disease. Particularly effective oligopeptide signaling molecules according to the invention in this context are GVLPALPQ (SEQ ID NO:33), LQGV (SEQ ID NO:1), MTRV (SEQ ID NO:42), VLPALPQVVC (SEQ ID NO:20), VLPALP (SEQ ID NO:3), VLPALPQ (SEQ ID NO:29), LPGCPRGVNPVVS (SEQ ID NO:40), LPGC (SEQ ID NO:41), VVCNYRDVRFESIRLPGCPRGVNPVVSYA VALSCQCAL (SEQ ID NO:35), CPRGVNPVVS (SEQ ID NO:50), which were shown herein to postpone onset of diabetes in a Non-obese Diabetic Mouse (NOD). Another approach to treatment of diabetes, in particular, insulin-independent diabetes (type 2), comprises inhibition of the PPARγ cascade with an oligopeptide signaling molecule or functional analogue or derivative thereof.

Another use that is provided relates to a method for combating or treating auto-immune disease. A non-limiting list of immune diseases includes:

Hashimoto's thyroiditis, primary myxedema thyrotoxicosis, pernicious anemia, autoimmune atrophic gastritis, Addison's disease, premature menopause, insulin-dependent diabetes mellitus, stiff-man syndrome, Goodpasture's syndrome, myasthenia gravis, male infertility, pemphigus vulgaris, pemphigoid, sympathetic ophthalmia, phagocogenic uveitis, multiple sclerosis, autoimmune hemolytic anemia, idiopathic thrombocytopenic purpura, idiopathic leucopenia, primary biliary cirrhosis, active chronic hepatitis, autoimmune deafness, cryptogenic cirrhosis, ulcerative colitis, Sjögren's syndrome, rheumatoid arthritis, dermatomyositis, polymyositis, scleroderma, mixed connective tissue disease, discoid lupus erythematosus, and systemic lupus erythematosus.

Another use that is provided relates to a method for combating or treating infections caused by microorganisms, in particular those infections that are caused by microorganisms that activate the NF-κB pathway during infections.

Such microorganisms are manifold, including bacteria, viruses, fungi, protozoa, but other pathogens (e.g., worms) can have the same effect. Activation of the NF-κB pathway by a microbial infection, in general, occurs via activation of the Toll-like receptor pathway. The invention provides a method to modulate and, in particular, to inhibit parts of gene expression that are related to the inflammatory responses of an organism that are generally activated through one of the Toll-like receptor pathways.

Toll-like receptor-mediated NF-κ-B activation is central in recognition of pathogens by a host. Such recognition of pathogens generally occurs through germline-encoded molecules, the so-called pattern recognition receptors (PRRs). These PRRs recognize widespread pathogen-associated molecular patterns (PAMPs). The pattern recognition receptors are expressed as either membrane-bound or soluble proteins. They include CD14, β2-integrins (CD11/CD18), C-type lectins, macrophage scavenger receptors, complement receptors (CR1/CD35, CR2/CD21) and Toll-like receptors (TLRs). TLRs are distinguished from other PRRs by their ability to recognize and discriminate between different classes of pathogens. TLRs represent a family of transmembrane proteins that have an extracellular domain comprising multiple copies of leucine-rich repeats (LRRs) and a cytoplasmic: Toll/IL-1R (TIR) motif that has significant homology to the intracellular signaling domain of the type I IL-1 receptor (IL-1RI). Therefore, TLRs are thought to belong to the IL-1R superfamily.

Pathogen-associated molecular patterns (PAMPS) are not expressed by hosts but are components of the pathogenic microorganism. Such PAMPS comprise bacterial cell wall components such as lipopolysaccharides (LPS), lipoproteins (BLP), peptidoglycans (PGN), lipoarabinomannan (LAM), lipoteichoic acid (LTA), DNA containing unmethylated CpG motifs, yeast and fungal cell wall mannans and beta-glucans, double-stranded RNA, several unique glycosylated proteins and lipids of protozoa, and so on.

Recognition of these PAMPS foremost provides for differential recognition of pathogens by TLRs. For example, TLR2 is generally activated in response to BLPs, PGNs of gram-positive bacteria, LAM of mycobacteria, and mannans of yeasts, whereas TLR4 is often activated by LPS of gram-negative bacteria and LTA of gram-negative bacteria; also a secreted small molecule MD-2 can account for TLR4 signaling.

Several oligopeptides capable of modulating gene expression according to the invention have earlier been tested, both ex vivo and in vivo, and in small animals, but a relationship with modulation of gene expression was not brought forward. A beneficial effect of these oligopeptides on LPS-induced sepsis in mice, namely the inhibition of the effect of the sepsis, was observed. Immunomodulatory effects with these oligopeptides have been observed in vitro and in ex vivo such as in T-cell assays showing the inhibition of pathological Th1 immune responses, suppression of inflammatory cytokines (MIF), increase in production of anti-inflammatory cytokines (IL-10, TGF-β) and immunomodulatory effects on antigen-presenting cells (APC) like dendritic cells, monocytes and macrophages.

Now that the insight has been provided that distinct synthetic oligopeptides or functional analogues or derivatives thereof, e.g., those that resemble breakdown products which can be derived by proteolysis from endogenous proteins such as hCG, can be used to modulate gene expression, e.g., by NF-κB inhibition, such oligopeptides find much wider application. Release of active NF-κB in cells is now known to occur after a variety of stimuli including treating cells with bacterial lipopolysaccharide (LPS) and the interaction with a Toll-like receptor (see, e.g., Guha and Mackman, Cell. Sign. 2001, 13:85-94). In particular, LPS stimulation of dendritic cells, monocytes and macrophages induces many genes that are under the influence of activation by transcription factors such as NF-κB, p50, EGR-1, IRF-1 and others that can be modulated by a signaling molecule according to the invention. Considering that LPS induction of EGR-1 is required for maximal induction of TNF-α, it is foreseen that inhibition of EGR-1 considerably reduces the effects of sepsis seen after LPS activation. Now knowing the gene modulatory effect of the signaling molecules such as oligopeptides as provided herein allows for rational design of signal molecule mixtures that better alleviate the symptoms seen with sepsis, one such mixture, a 1:1:1 mixture of LQGV (SEQ ID NO:1), (SEQ ID NO:2) and VLPALP (SEQ ID NO:3), was administered to primates in a gram-negative-induced rhesus monkey sepsis model for prevention of septic shock and found to be effective in this primate model. Accordingly, the invention provides a pharmaceutical composition for the treatment of sepsis in a primate and a method for the treatment of sepsis in a primate comprising subjecting the primate to a signaling molecule according to the invention, preferably to a mixture of such signaling molecules. Administration of such a signaling molecule or mixture preferably occurs systematically, e.g., by intravenous or intraperitoneal administration. In a further embodiment, such treatment also comprises the use of, e.g., an antibiotic, however, only when such use is not contra-indicated because of the risk of generating further toxin loads because of lysis of the bacteria subject to the action of those antibiotics in an individual thus treated.

Another use that is contemplated relates to a method for combating or treating viral infections, in particular those infections that are caused by viruses that activate the NF-κB pathway during infections. Such virus infections are manifold; classical examples are hepatitis B virus-induced cell transformation by persistent activation of NF-κB. Use of a signaling molecule according to the invention is herein provided to counter or prevent this cell transformation.

Other diseases where persistent NF-κB activation may be advantageously inhibited by a signaling molecule include transplantation-related diseases such as transplantation-related immune responses, graft-versus-host disease, in particular with bone marrow transplants, acute or chronic xeno-transplant rejection, and post-transfusion thrombocytopenia.

Another case where persistent NF-κB activation is advantageously inhibited by a signaling molecule is found in the prevention or mitigation of ischemia-related tissue damage seen after infarcts, seen, e.g., in vivo in brain or heart, or ex vivo in organs or tissue that are being prepared or stored in preparation of further use as a transplant. Ischemia-related tissue damage can now be mitigated by perfusing the (pre)ischemic area with a signaling molecule according to the invention that inhibits NF-κB activation. An example of a condition where ischemia (also called underperfusion) plays a role is eclampsia, which can be ascribed to focal cerebral ischemia resulting from vasoconstriction, consistent with the evidence of changes detected by new cerebral imaging techniques. The liver dysfunction intrinsic to the HELLP (hemolysis, elevated liver enzymes, and low platelet count) syndrome could also be attributed to the effects of acute underperfusion. Other conditions of ischemia are seen after coronary occlusion, leading to irreversible myocardial damage produced by prolonged episodes of coronary artery occlusion and reperfusion in vivo, which has already been discussed in International Patent Appln. PCT/NL01/00259 as well.

A medical practitioner can readily diagnose ischemia-reperfusion injury. For example, to differentiate it from early acute rejection in a kidney transplant recipient, a kidney biopsy should be able to differentiate between early acute rejection and other pathologies, including ischemia-reperfusion-associated acute tubular necrosis (“ATN”). The biopsy findings for acute rejection would include tubulitis and other findings as described in the Banff criteria. The absence of findings consistent with acute rejection would rule it out; other histopathologic findings of ATN would be seen.

The invention also relates to the treatment of ischemic-reperfusion injury such as seen after an ischemic event seen with stroke, myocardial infarction, extensive bleeding or embolic events, in general after the disruption of blood flow to (parts) of an organ.

An ischemic event refers to an event in which the blood supply to a tissue is obstructed. Due to this obstruction, the endothelial tissue lining the affected blood vessels becomes “sticky” and begins to attract circulating white blood cells. The white cells bound to the endothelium eventually migrate into the affected tissue, causing significant tissue destruction. Although neither acute myocardial infarction nor stroke is directly caused by inflammation, much of the underlying pathology and the damage that occurs after an acute ischemic event are caused by acute inflammatory responses during reperfusion, the restoration of blood flow to the affected organ. Early restitution of blood flow to ischemic tissues is essential to halt the progression of cellular injury associated with decrease of oxygen supply and nutrient delivery. This fact provides the basis for the traditional view that minimizing ischemic time is the only important intervention for diminishing the extent of ischemic injury. However, it is now well recognized that reperfusion of ischemic tissues initiates a complex series of reactions that can paradoxically injure tissues. Although several mechanisms have been proposed to explain the pathogenesis of ischemia-reperfusion injury, most attention has focused on a role for reactive oxygen and nitrogen metabolites and inflammatory leukocytes. In addition to the local tissue injury, distant organs can also be affected, particularly if the intensity of the inflammatory reaction in post-ischemic tissue (e.g., intestine) is great. The remote effects of ischemia-reperfusion injury are most frequently observed in the lung and (cardio- or cerebro-)vascular system, and can result in the development of the systemic inflammatory response syndrome (SIRS) and multiple organ dysfunction syndrome (MODS), both of which account for 30-40% of the mortality in tertiary referral intensive care units (ICUs). This application, however, mostly deals with localized or systemic ischemic events and the reperfusion damage seen thereafter.

In WO 01/72831 the inventors provide a method and a pharmaceutical composition for modulating cardiovascular or circulatory disorders, such as heart failure, brain infarctions, Alzheimer's disease, thrombosis, arteriosclerosis, pregnancy-related cardiovascular or circulatory disorders and the like. It has been found that an immunoregulator as described in the application has a very beneficial effect on animals, including humans, suffering from a cardiovascular disorder. The immunoregulator according to WO 01/72831 also widens the scope of possibilities of dotter treatments. In cases where conventionally such a treatment could not be performed because of risks of an oxygen tension becoming too low, a dotter treatment in cases of myocardial infarction is feasible when combined with treatment with the immunoregulator. Accordingly, expensive and difficult bypass surgery may in many cases be avoided, and the application also suggested the same protective effect of the immunoregulator in other organs as well in circulatory related disease.

The current invention provides additional modes and means of treatment. The invention provides a method for modulating an ischemic event in a subject believed to be in need thereof comprising providing the subject with a signaling molecule comprising a short gene-regulatory peptide or functional analogue thereof, wherein the signaling molecule is administered in an amount sufficient to modulate the ischemic event. The signal molecule is preferably a short peptide, preferably of at most 30 amino acids long, or a functional analogue or derivative thereof. In a much preferred embodiment, the peptide is an oligopeptide of from about 3 to about 15 amino acids long, preferably 4 to 12, more preferably 4 to 9, most preferably 4 to 6 amino acids long, or a functional analogue or derivative thereof. Of course, such signaling molecule can be longer, for example by extending it (N- and/or C-terminally), with more amino acids or other side groups, which can for example be (enzymatically) cleaved off when the molecule enters the place of final destination. In particular, a method is provided wherein the signaling molecule modulates translocation and/or activity of a gene transcription factor. It is particularly useful when the gene transcription factor comprises an NF-κB/Rel protein or an AP-1 protein. Ischemia induces increased expression of inflammatory cytokines due to activation of NF-κB and AP-1, and in a preferred embodiment the invention provides a method translocation and/or activity of the NF-κB/Rel protein is inhibited. In one embodiment, the peptide is selected from the group of peptides LQG, AQG, LQGV (SEQ ID NO:1), (SEQ ID NO:2), LQGA (SEQ ID NO:19), VLPALP (SEQ ID NO:3), ALPALP (SEQ ID NO:21), VAPALP (SEQ ID NO:22), ALPALPQ (SEQ ID NO:23), VLPAAPQ (SEQ ID NO:24), VLPALAQ (SEQ ID NO:25), LAGV (SEQ ID NO:26), VLAALP (SEQ ID NO:27), VLPALA (SEQ ID NO:28), VLPALPQ (SEQ ID NO:29), VLAALPQ (SEQ ID NO:30), VLPALPA (SEQ ID NO:31), GVLPALP (SEQ ID NO:32), LQGVLPALPQVVC (SEQ ID NO:34), LPGCPRGVNPVVS (SEQ ID NO:40), LPGC (SEQ ID NO:41), MTRV (SEQ ID NO:42), MTR, VVC.

Now that the insight has been provided that distinct synthetic oligopeptides, e.g., those that resemble breakdown products which can be derived by proteolysis from endogenous proteins such as hCG, can be used to modulate gene expression, e.g., by NF-κB inhibition, the oligopeptides find much wider application. For example, the invention provides a method for perfusing a transplant with a perfusing fluid comprising at least one signaling molecule according to the invention; ischemic or pre-implantation damage due to activation of NF-κB in the transplant can then greatly be diminished, allowing a wider use of the transplants.

In certain embodiments, the invention provides a signaling molecule useful in modulating expression of a gene in a cell. Several examples of the use of such a signaling molecule for treating medical or veterinary conditions are herewith given. In one embodiment, the invention provides such use in the treatment of an immune-mediated disorder, in particular of those cases wherein a central role of NF-κB/Rel proteins in the immune response is found. However as said, modulating gene expression via modulating activity of other transcription factors, such as AP-1 or PPARγ, and others, is also provided, now that the gene modulating role of signaling molecules such as the oligopeptides or analogues or derivatives thereof is understood. As also said, now knowing that oligopeptides, likely breakdown products, play such a central role in modulation of gene expression, the invention provides straightforward ways for identifying further gene expression modulating oligopeptides, and provides synthetic versions of these, and analogues and derivatives thereof for use in a wide variety of disorders and for use in the preparation of a wide variety of pharmaceutical compositions. Examples of such treatment and useful pharmaceutical compositions are found in relation to conditions wherein the immune-mediated disorder comprises chronic inflammation, such as diabetes, multiple sclerosis or acute or chronic transplant rejection, in particular in those cases whereby antigen-presenting cells (APCs) or dendritic cells (DCs) are enhanced by (overactive) and persistent NF-κB-expression or wherein the immune-mediated disorder comprises acute inflammation, such as septic or anaphylactic shock or acute transplant rejection. Other immune-mediated disorders that can be treated with a pharmaceutical composition comprising a signaling molecule according to the invention comprise auto-immune disease, such as systemic lupus erythematosus or rheumatoid arthritis (in particular by inhibiting IL-8 and/or IL-15 production by inhibiting NF-κB activity on the expression of these genes), allergy, such as asthma or parasitic disease, overly strong immune responses directed against an infectious agent, such as a virus or bacterium (in particular responses that include rapid hemorrhagic disease caused by infection with organisms such as Yersinia pestis, Ebola-virus, Staphylococcus aureus (e.g., in cases of toxic shock syndrome), bacterial (such as meningococcal) or viral meningitis and/or encephalitis, and other life-threatening conditions). Such overly strong responses are seen with, e.g., pre-eclampsia, recurrent spontaneous abortions (RSA) or preterm parturition or other pregnancy-related disorders. Especially with forms of eclampsia/pre-eclampsia that are associated with genetically programmed increased production of tumor-growth factor beta-1, treatment according to the invention is recommended. Also, in situations where RSA is likely attributable to increased IL-10 levels during pregnancy, or to increased TNF-α activity, e.g., due to the presence of an unfavorable allele, in particular of a G to A polymorphism in the promoter of the gene encoding TNF-α, treatment with a pharmaceutical composition as provided herein is recommended. Treatment directed at such pregnancy-related immune disorders is herein also provided by inhibiting NF-κB activity directed at activating natural killer (NK) cell activity. Also, LPS-induced reduced fertility, or abortions, seen in pregnant sows, can be reduced by applying a signaling molecule or method as provided herein.

Such use in treatment of an immune-mediated disorder preferably comprises regulating relative ratios and/or cytokine activity of lymphocyte-, dendritic- or antigen-presenting cell subset-populations in a treated individual, in particular wherein the subset populations comprise Th1 or Th2, or DC1 or DC2 cells. Other embodiments of the invention comprise use of a signaling molecule according to the invention for the manufacture of a medicament for modulating a cardiovascular or circulatory disorder, such as coronary arterial occlusion, and also in a pregnancy-related cardiovascular or circulatory disorder.

Furthermore, the invention provides a pharmaceutical composition for modulating a cardiovascular or circulatory disorder, in particular a pregnancy-related cardiovascular or circulatory disorder, comprising a signaling molecule according to the invention or mixtures thereof. Such a composition finds use in a method for modulating a cardiovascular or circulatory disorder, in particular a pregnancy-related cardiovascular or circulatory disorder, comprising subjecting an animal (in particular a mammal) to treatment with at least one signaling molecule according to the invention. Non-pregnancy-related disorders, which are, e.g., related to hypercholesterolemia, are susceptible to treatment with a signaling molecule according to the invention as well. For example, apolipoprotein E (apoE) deficiency is associated with a series of pathological conditions including dyslipidemia, atherosclerosis, Alzheimer's disease, increased body weight and shorter life span. Inheritance of different alleles of the polymorphic apoE gene is responsible for 10% of the variation in plasma cholesterol in most populations. Individuals homozygous for one variant, apoE2, can develop type III dysbetalipoproteinemia if an additional genetic or environmental factor is present. Some much rarer alleles of apoE produce dominant expression of this disorder in heterozygous individuals. ApoE is a ligand for the LDL receptor and its effects on plasma cholesterol are mediated by differences in the affinity of the LDL receptor for lipoproteins carrying variant apoE proteins. The factors that regulate apoE gene transcription have been investigated extensively by the expression of gene constructs in transgenic mice and involve complex interactions between factors that bind elements in the 5′ promoter region, in the first intron and in 3′ regions many kilobases distant from the structural gene. Deletion of the ApoE gene is associated with changes in lipoprotein metabolism (plasma total cholesterol), HDL cholesterol, HDL/TC, and HDL/LDL ratios, esterification rate in apo B-depleted plasma, plasma triglyceride, hepatic HMG-CoA reductase activity, hepatic cholesterol content, decreased plasma homocyst(e)ine and glucose levels, and severe atherosclerosis and cutaneous xanthomatosis. The invention provides a method and a signaling molecule for the treatment of conditions that are associated with dysfunctional LDL receptors such as apoE and other members of the apolipoprotein family. In particular, use of a signaling molecule comprising GVLPALPQ (SEQ ID NO:33) and/or VLPALP (SEQ ID NO:3) or a functional analogue or derivative thereof is preferred.

The invention also provides use of a signaling molecule for the preparation of a pharmaceutical composition or medicament and methods of treatment for various medical conditions that are other than use in the preparation of a pharmaceutical composition for the treatment of an immune-mediated disorder or a method of treatment of an immune-mediated-disorder. For example, the invention provides topical application in an ointment or spray comprising a signaling molecule according to the invention, for the prevention or mitigation of skin afflictions, such as eczemas, psoriasis, but also of skin damage related to over-exposure to UV-light.

Also, use is contemplated in palliative control, whereby a gene related to prostaglandin synthesis is modulated such that COX2 pathways are affected.

In certain embodiments, the invention provides use of a signaling molecule for the preparation of a pharmaceutical composition or medicament and methods of treatment for various medical conditions that are other than use in the preparation of a pharmaceutical composition for the treatment of wasting syndrome, such as the treatment of particular individuals that are suffering from infection with HIV or a method of treatment of wasting syndrome of such individuals.

In certain embodiments, the invention provides the use of a signaling molecule according to the invention for the preparation of a pharmaceutical composition or medicament for modulating angiogenesis or vascularization, in particular during embryonal development or after transplantation, to stimulate vascularization into the transplanted organ or inhibit it in a later phase. Signaling molecules that effect angiogenesis are disclosed herein in the detailed description.

Use as provided herein also comprises regulating TNF-α receptor (e.g., CD27) expression on cells, thereby modulating the relative ratios and/or cytokine activity of lymphocyte-, dendritic- or antigen-presenting cell subset populations in a treated individual. As, e.g., described in the detailed description, a particular oligopeptide according to the invention is capable of down-regulating CD27 expression on cells of the T-cell lineage.

Down-regulating TNF-α itself is also particularly useful in septic-shock-like conditions that not only display increased TNF-α activity but display further release of other inflammatory compounds, such as NO. NO production is a central mediator of the vascular and inflammatory response. Our results show that inflammatory cells like macrophages stimulated with an inflammatory active compound such as LPS produce large amounts of NO. However, these cells co-stimulated with most of the NMPF peptides (NMPF peptides 1 to 14, 43 to 66 and 69), even in a very low dose (1 pg/ml), inhibited production of NO. Typical septic-shock-like conditions that can preferably be treated by down-regulating TNF-α and NO production comprise disease conditions such as those caused by Bacillus anthracis and Yersinia pestis toxins or infections with these microorganisms likely involved in bioterrorism. Anthrax toxin is produced by B. anthracis, the causative agent of anthrax, and is responsible for the major symptoms of the disease. Clinical anthrax is rare, but there is growing concern over the potential use of B. anthracis in biological warfare and terrorism. Although a vaccine against anthrax exists, various factors make mass vaccination impractical. The bacteria can be eradicated from the host by treatment with antibiotics, but because of the continuing action of the toxin, such therapy is of little value once symptoms have become evident. Thus, a specific inhibitor of the toxin's action will prove a valuable adjunct to antibiotic therapy. The toxin consists of a single receptor-binding moiety, termed “protective antigen” (PA), and two enzymatic moieties, termed “edema factor” (EF) and “lethal factor” (LF). After release from the bacteria as nontoxic monomers, these three proteins diffuse to the surface of mammalian cells and assemble into toxic, cell-bound complexes.

Cleavage of PA into two fragments by a cell-surface protease enables the fragment that remains bound to the cell, PA63, to heptamerize and bind EF and LF with high affinity. After internalization by receptor-mediated endocytosis, the complexes are trafficked to the endosome. There, at low pH, the PA moiety inserts into the membrane and mediates translocation of EF and LF to the cytosol. EF is an adenylate cyclase that has an inhibitory effect on professional phagocytes, and LF is a protease that acts specifically on macrophages, causing their death and the death of the host.

Down-regulating TNF-α itself, and/or a receptor for TNF-α, as is herein also provided, is also beneficial in individuals with Chagas cardiomyopathy.

Also, use of a signaling molecule for modulation of vascularization or angiogenesis in wound repair, in particular of burns, is herein provided. Also, use of a pharmaceutical composition as provided herein is provided in cases of post-operative physiological stress, whereby not only vascularization will benefit from treatment, but the general well-being of the patient is improved as well.

Another use of a signaling molecule according to the invention comprises its use for the preparation of another pharmaceutical composition for the treatment of cancer. Such a pharmaceutical composition preferably acts via modulating and up-regulating apoptotic responses that are classically down-regulated by NF-κB activity. Inhibiting the activity with a signaling molecule according to the invention allows for increased cell death of tumorous cells. Another anti-cancerous activity of a signaling molecule as provided herein comprises down-regulation of c-myb, in particular, in the case of hematopoietic tumors in humans. In this context, down-regulation of 14.3.3 protein is also provided.

A further use of a signaling molecule according to the invention comprises its use for the preparation of a further pharmaceutical composition for the treatment of cancer. Such a pharmaceutical composition preferably acts via modulating and down-regulating transferrin receptor availability, in particular, on tumorous cells. Transferrin receptors are classically up-regulated by NF-κB activity. Inhibiting the activity with a signaling molecule according to the invention allows for reduced iron up-take and increased death of tumorous cells. In particular, erythroid and thromboid cells are susceptible to the treatment.

Yet a further use of a signaling molecule according to the invention comprises its use for the preparation of yet another pharmaceutical composition for the treatment of cancer, in particular of cancers that are caused by viruses, such as is the case with retroviral-induced malignancies and other viral-induced malignancies. Such a pharmaceutical composition preferably acts via modulating and down-regulating cell-proliferative responses that are classically up-regulated by virus-induced transcriptional or NF-κB activity. Inhibiting the activity with a signaling molecule according to the invention allows for decreased proliferation and increased cell death of tumorous cells. Such a pharmaceutical composition may also act via modulating angiogenic responses induced by IL-8, whereby, e.g., inhibition of IL-8 expression via inhibition of transcription factor AP-1 or NF-κB expression results in the inhibition of vascularization-dependent tumor growth.

Furthermore, the invention provides the use of a signaling molecule for the preparation of a pharmaceutical composition for optimizing human or animal fertility and embryo survival, and a method for optimizing fertility and embryo survival. In particular, the invention provides for a method and composition allowing the down-regulation of TNF-α in the fertilized individual, optimally in combination with a composition and method for up-regulating IL-10 in the individual. Such a composition and method find immediate use in both human and veterinary medicine.

Also, the invention provides the use of a signaling molecule for the preparation of a pharmaceutical composition for modulating the body weight of an individual, in particular, by modulating gene expression of a gene under influence of peroxisome proliferator-activated receptor gamma (PPARγ) activation and lipid metabolism by applying a signaling molecule according to the invention, and a method for modulating body weight comprising providing an individual with a signaling molecule according to the invention.

A further use of a signaling molecule as provided herein lies in the modulation of expression of a gene in a cultured cell. Such a method as provided herein comprises subjecting a signaling molecule according to the invention to the cultured cell. Proliferation and/or differentiation of cultured cells (cells having been or being under conditions of in vitro cell culture known in the art) can be modulated by subjecting the cultured cell to a signaling molecule according to the invention. It is contemplated that, e.g., research into proliferation or differentiation of cells, such as stem-cell research, will benefit greatly from the understanding that a third major way of effecting gene modulation exists, and considering the ease of application of synthetic peptides and analogues or derivatives thereof.

Furthermore, it is contemplated that a signaling molecule as provided herein finds an advantageous use as a co-stimulatory substance in a vaccine, accompanying modern day adjuvants or replacing the classically used mycobacterial adjuvants, especially considering that certain mycobacteria express hCG-like proteins, of which it is now postulated that these bacteria have already made use of this third pathway found in gene modulation as provided herein by providing the host with breakdown products mimicking the signaling molecules identified herein. Treatment and use of the compositions as provided herein is not restricted to animals only; plants and other organisms are also subject to this third pathway as provided herein. Furthermore, now that the existence of such a pathway has been demonstrated, it is herein provided to make it the subject of diagnosis as well, e.g., to determine the gene modulatory state of a cell in a method comprising determining the presence or absence of a signaling molecule as provided herein or determining the presence or absence of a protease capable of generating such a signaling molecule from a (preferable endogenous) protein.

The invention in particular relates to the treatment of ischemic-reperfusion injury such as seen after an ischemic event seen with stroke, myocardial infarction, extensive bleeding or embolic events, in general after the disruption of blood flow to (parts) of an organ.

An ischemic event refers to an event in which the blood supply to a tissue is obstructed. Due to this obstruction, the endothelial tissue lining the affected blood vessels becomes “sticky” and begins to attract circulating white blood cells. The white cells bound to the endothelium eventually migrate into the affected tissue, causing significant tissue destruction. Although neither acute myocardial infarction nor stroke is directly caused by inflammation, much of the underlying pathology and the damage that occurs after an acute ischemic event are caused by acute inflammatory responses during reperfusion, the restoration of blood flow to the affected organ. Early restitution of blood flow to ischemic tissues is essential to halt the progression of cellular injury associated with decrease of oxygen supply and nutrient delivery. This fact provides the basis for the traditional view that minimizing ischemic time is the only important intervention for diminishing the extent of ischemic injury. However, it is now well recognized that reperfusion of ischemic tissues initiates a complex series of reactions that can paradoxically injure tissues. Although several mechanisms have been proposed to explain the pathogenesis of ischemia-reperfusion injury, most attention has focused on a role for reactive oxygen and nitrogen metabolites and inflammatory leukocytes. In addition to the local tissue injury, distant organs can also be affected, particularly if the intensity of the inflammatory reaction in post-ischemic tissue (e.g., intestine) is great. The remote effects of ischemia-reperfusion injury are most frequently observed in the lung and (cardio- or cerebro-)vascular system, and can result in the development of the systemic inflammatory response syndrome (SIRS) and multiple organ dysfunction syndrome (MODS), both of which account for 30-40% of the mortality in tertiary referral intensive care units (ICUs). This application, however, mostly deals with localized or systemic ischemic events and the reperfusion damage seen thereafter.

In WO 01/72831 the inventors provide a method and a pharmaceutical composition for modulating cardiovascular or circulatory disorders, such as heart failure, brain infarctions, Alzheimer's disease, thrombosis, arteriosclerosis, pregnancy-related cardiovascular or circulatory disorders and the like. It has been found that an immunoregulator as described in the application has a very beneficial effect on animals, including humans, suffering from a cardiovascular disorder. The immunoregulator according to WO 01/72831 also widens the scope of possibilities of dotter treatments. In cases where conventionally such a treatment could not be performed because of risks of an oxygen tension becoming too low, a dotter treatment in cases of myocardial infarction is feasible when combined with treatment with the immunoregulator. Accordingly, expensive and difficult bypass surgery may in many cases be avoided, and the application also suggested the same protective effect of the immunoregulator in other organs as well in circulatory related disease.

The current invention provides additional modes and means of treatment of ischemia-reperfusion injury and reperfusion injury. The invention provides a method for modulating an ischemia-reperfusion injury in a subject believed to be in need thereof comprising providing the subject with a signaling molecule comprising a short gene-regulatory peptide or functional analogue thereof, wherein the signaling molecule is administered in an amount sufficient to modulate the ischemia-reperfusion injury. The signal molecule is preferably a short peptide, preferably of at most 30 amino acids long, or a functional analogue or derivative thereof. In a much preferred embodiment, the peptide is an oligopeptide of from about 3 to about 15 amino acids long, preferably 4 to 12, more preferably 4 to 9, most preferably 4 to 6 amino acids long, or a functional analogue or derivative thereof. Of course, such signaling molecule can be longer, for example by extending it (N- and/or C-terminally), with more amino acids or other side groups, which can, for example, be (enzymatically) cleaved off when the molecule enters the place of final destination. In particular, a method is provided wherein the signaling molecule modulates translocation and/or activity of a gene transcription factor. It is particularly useful when the gene transcription factor comprises an NF-κB/Rel protein or an AP-1 protein. Ischemia induces increased expression of inflammatory cytokines due to activation of NF-κB and AP-1, and in a preferred embodiment the invention provides a method translocation and/or activity of the NF-κB/Rel protein is inhibited. In one embodiment, the peptide is selected from the group of peptides LQG, AQG, LQGV (SEQ ID NO:1), (SEQ ID NO:2), LQGA (SEQ ID NO:19), VLPALP (SEQ ID NO:3), ALPALP (SEQ ID NO:21), VAPALP (SEQ ID NO:22), ALPALPQ (SEQ ID NO:23), VLPAAPQ (SEQ ID NO:24), VLPALAQ (SEQ ID NO:25), LAGV (SEQ ID NO:26), VLAALP (SEQ ID NO:27), VLPALA (SEQ ID NO:28), VLPALPQ (SEQ ID NO:29), VLAALPQ (SEQ ID NO:30), VLPALPA (SEQ ID NO:31), GVLPALP (SEQ ID NO:32), LQGVLPALPQVVC (SEQ ID NO:34), LPGCPRGVNPVVS (SEQ ID NO:40), LPGC (SEQ ID NO:41), MTRV (SEQ ID NO:42), MTR, VVC.

### BRIEF DESCRIPTION OF FIGURES

FIGS. 1 and 2. Bone marrow (BM) cell yield of treated BALB/c mice (n=6). BM cells were isolated from treated mice and cultured in vitro in the presence of rmGM-CSF for nine days. These figures show cell yield after nine days of culture of BM cells isolated from mice treated with PBS, LPS or LPS in combination with NMPF peptides 4, 46, 7 and 60. In these figures, cell yield is expressed in relative percentage of cells compared to PBS. Each condition consists of six Petri dishes and results shown in these figures are representative of six dishes. Differences of ≧20% were considered significant and line bars represent significant data as compared to the LPS control group. A representative experiment is shown. Findings involving all experimental conditions were entirely reproduced in three additional experiments.

FIG. 3. Effect of ex vivo treatment on MHC-II expression on CD11c⁺ cells. Bone marrow (BM) cells were isolated from treated BALB/c mice (n=6) and cultured ex vivo in the presence of rmGM-CSF for nine days. This figure shows MHC-II expression expressed in mean fluorescence intensity (MFI) after nine days of culturing of BM cells isolated from PBS, LPS or LPS in combination with NMPF. Each condition consists of six Petri dishes and results shown in these figures are representative of six dishes. Differences of ≧20% were considered significant and line bars represent significant data as compared to LPS control group. A representative experiment is shown. Findings involving all experimental conditions were entirely reproduced in three additional experiments.

FIGS. 4 through 7. Bone marrow (BM) cell yield of in vitro-treated BM cultures. BM cells from BALB/c mice (n=3) were cultured in vitro and treated with either PBS, LPS (t=6), NMPF-4, 7, 46, 60 (t=0 or t=6) or combination of NMPF with LPS (t=6), in the presence of rmGM-CSF for nine days. These figures show cell yield expressed in relative percentage of cells compared to PBS after nine days of culture of BM cells. Each condition consists of six Petri dishes and results shown in these figures are representative of six dishes. Differences of ≧20% were considered significant. Line bars represent significant data as compared to LPS control group and dotted bars represent significant data as compared to PBS group. A representative experiment is shown. Findings involving all experimental conditions were entirely reproduced in three additional experiments.

FIGS. 8 through 11. Effect of in vitro treatment on MHC-II expression on CD11c⁺ cells. BM cells from BALB/c mice (n=3) were cultured in vitro and treated with either PBS, LPS (t=6), NMPF-4, 7, 46, 60 (t=0) or t=6 days) or a combination of NMPF with LPS (t=6 days), in the presence of rmGM-CSF for nine days. These figures show MHC-II expression expressed in mean fluorescence intensity (MFI) of CD11c positive cells after nine days of culturing of BM cells. Each condition consists of six Petri dishes, and results shown in these figures are representative of six dishes. Differences of ≧20% were considered significant. Line bars represent significant data as compared to LPS control group and dotted bars represent significant data as compared to PBS group. A representative experiment is shown. Findings involving all experimental conditions were entirely reproduced in three additional experiments.

FIGS. 12 through 15. Bone marrow (BM) cell yield of ex vivo-treated BALB/c mice (n=6). BM cells were isolated from treated mice and cultured ex vivo in the presence of rmGM-CSF for nine days. These figures show cell yield after nine days of culture of BM cells in suspension (unattached) and attached to Petri dish (attached). BM cells were isolated from mice treated with PBS, LPS or LPS in combination with different NMPF peptides. In these figures cell yield is expressed in relative percentage of cells compared to PBS. Each condition consists of six Petri dishes and results shown here are representative of six dishes. Differences of ≧20% were considered significant and line bars represent significant data as compared to LPS control group. A representative experiment is shown. Findings involving all experimental conditions were entirely reproduced in three additional experiments.

FIGS. 16 and 17. Bone marrow (BM) cell yield of in vitro treated BM cultures from NOD mice. BM cells from 15-week-old female NOD mice (n=3) were cultured in vitro and treated with either PBS or NMPF in the presence of rmGM-CSF for nine days. These figures show cell yield after nine days of culture of BM cells in suspension (unattached) and attached to Petri dishes (attached). In these figures cell yield is expressed in relative percentage of cells compared to PBS. Each condition consists of six Petri dishes and results shown here are representative of six dishes. Differences of ≧20% were considered significant and dotted bars represent significant data as compared to PBS control group. A representative experiment is shown. Findings involving all experimental conditions were entirely reproduced in three additional experiments.

FIGS. 18 through 30. In vivo treatment of fertilized chicken eggs with NMPF and the effect of NMPF on angiogenesis. Fertile chicken eggs (day 0) were treated with either PBS, NMPF, VEGF or VEGF in combination with NMPF. Ten eggs were injected for every condition. On day 8 of incubation, the embryos were removed from the eggs and were placed in a 100-mm Petri dish. The embryo and the blood vessels were photographed in vivo with the use of a microscope. Of each egg one overview picture was taken and four detail pictures of the blood vessels were taken. Quantification of angiogenesis (vessel branches) was accomplished by counting the number of blood vessel branches. Quantification of this vasculogenesis was accomplished by measuring the blood vessel thickness. The number of blood vessel branches and vessel thickness were measured in the pictures and were correlated to a raster (in the pictures) of 10 mm² for comparison. The mean number of branches and the mean blood vessel thickness of each condition (N=10) were calculated and compared to either the PBS or VEGF controls using a Student's T-test. Line bars represent significant (p<0.05) data as compared to PBS control group and dotted bars represent significant (p<0.05) data as compared to VEGF group. FIGS. 20 through 30 show the effect of NMPF on vessel branches. FIGS. 29 and 30 show the effect of NMPF on vessel thickness.

FIG. 31. Detection of NF-κB via EMSA. This figure shows the presence of NF-κB in the nuclear extracts of RAW264.7 cells treated with LPS or NMPF in combination with LPS for four hours. Numbers 1 through 13 correspond to nuclear extracts from cells treated with NMPF and LPS. CTL corresponds to nuclear extracts from cells treated with LPS only. Specificity of the radioactively labeled NF-κB probe is shown by competition with the unlabeled oligonucleotide (u1, u2, u3) in three different concentrations (1×, 10×, 100×) with nuclear extracts of CTL and olg corresponding to samples containing only labeled oligonucleotide (without nuclear extract). Description: (NMPF-1)VLPALPQVVC (SEQ ID NO:20), (NMPF-2)LQGVLPALPQ (SEQ ID NO:49), (NMPF-3)LQG, (NMPF-4)LQGV (SEQ ID NO:1), (NMPF-5)GVLPALPQ (SEQ ID NO:33), (NMPF-6)VLPALP (SEQ ID NO:3), (NMPF-7)VLPALPQ (SEQ ID NO:29), (NMPF-8)GVLPALP (SEQ ID NO:32), (NMPF-9)VVC, (NMPF-11)MTRV (SEQ ID NO:42), (NMPF-12)MTR.

FIG. 32. HPLC chromatograph (wavelength 206) in which data profiles obtained from the nuclear protein extracts of LPS and LPS in combination with NMPF-stimulated RAW264.7 cells are overlayed.

FIG. 33. MSn analysis of NMPF-4 peptide.

FIG. 34. MSn analysis of fraction from nuclear extract of LPS-stimulated RAW264.7 cells. Upper panel shows full spectrum of the fraction and lower panel shows the MS/MS spectrum of mass 413.13.

FIG. 35. MSn analysis of fraction from nuclear extract of LPS in combination with NMPF-4-stimulated RAW264.7 cells. Upper panel shows full spectrum of the fraction and lower panel shows the MS/MS spectrum of mass 416.07.

FIGS. 36 through 47. Effect of NMPF on septic shock syndrome in Rhesus monkeys. On the time point 70 minutes, E. coli was infused, and at the end of E. coli infusion (time point 190 minutes), the antibiotic Baytril was injected. The control monkey (monkey 429) was treated with 0.9% NaCl at the time point of 100 minutes, whereas the NMPF-treated monkeys (monkeys 459 and 427) received the NMPF treatment at the same time point as the control monkey. Heart rate (beats per minute), blood pressure (mmHg), difference between systolic and diastolic blood pressure and blood oxygen concentration (saturation in %) of the control monkey 429 (FIGS. 36 through 39), and NMPF-treated monkeys 459 (FIGS. 40 through 43) and 427 (FIGS. 44 through 47) in the time (minutes) during the experiment are shown.

FIG. 48. This figure shows the NO production of LPS-(10 μg/ml) stimulated RAW 264.7 macrophages co-stimulated with different NMPF peptides (1 pg/ml).

FIG. 49. The figure shows the NO production of LPS-(10 μg/ml) stimulated RAW 264.7 macrophages co-stimulated with different NMPF peptides with three different concentrations.

FIG. 50. This figure shows the percentage of diabetic NOD mice treated for two weeks with the various NMPF peptides.

FIG. 51. This figure shows the performed glucose tolerance test (GTT) in NOD mice treated with NMPF peptides (A), and fasting blood glucose levels (B).

FIG. 52. Hemorrhagic Shock model (HS) (*=time of administration peptide A, B or C in the peptide groups)

FIG. 53. Mean Arterial Pressure in Hemorrhagic Shock model in sham, shock, and Peptide A, B and C experiments.

FIG. 54. Hematocrit in Hemorrhagic Shock model in (from left to right) sham, shock, and Peptide A, B and C experiments.

FIG. 55. Leukocytes in Hemorrhagic Shock model during sham, shock, Peptide A, B and C experiments.

FIG. 56. Macrophages (MO) and granulocytes (GR) in Hemorrhagic Shock model in (from left to right) sham, trauma-hemorrhagic shock, and Peptide A, B and C experiments.

FIG. 57. Arterial blood flow in Hemorrhagic Shock model in (from left to right) sham, shock, and Peptide A, B and C experiments.

### DETAILED DESCRIPTION OF THE INVENTION

Cells react to environmental and intrinsic changes, which they perceive through extracellular and inter- as well as intracellular signals. The nature of these signals can be either, e.g., physical or chemical. Moreover, different classes of molecules present in blood react to each other and induce a cascade of reactions that have direct effect on other molecules and/or eventually lead to cellular responses, e.g., complement system and blood coagulation proteins.

Many genes are regulated not by a signaling molecule that enters the cells but by molecules that bind to specific receptors on the surface of cells, e.g., receptors with enzymatic activity (receptor tyrosine kinases, receptor-like protein tyrosine phosphatases, receptor serine/threonine kinases, histidine kinases, guanylyl cyclases) and receptors without enzymatic activity (cytokine receptors, integrins, and G-protein-coupled receptors). Interaction between cell-surface receptors and their ligands can be followed by a cascade of intracellular events that modulate one or more intracellular-transducing proteins, including variations in the intracellular levels of so-called second messengers (diacylglycerol, Ca²⁺, cyclic nucleotides, inositol(1,4,5) trisphosphate, phosphatidylinositol(3,4,5) trisphosphate, and phosphatidylinositol transfer protein (PITP)). This leads to the activation or inhibition of a so-called “effector protein.” The second messengers in turn lead to changes in protein, e.g., protein phosphorylation through the action of cyclic AMP, cyclic GMP, calcium-activated protein kinases, or protein kinases (for example, AGC group serine/threonine protein kinases, CAMK group serine/threonine protein kinases, CMGC group serine/threonine kinases, protein tyrosine kinase group, or others like MEK/Step 7p). Phosphorylation by protein kinases is one of the regulatory mechanisms in signal transmission that modulate different cellular pathways such as Ras/MAPK pathway, MAP kinase pathway, JAK-STAT pathway, or wnt-pathway. In many instances, this all results in altered gene expression (for example, genes for the regulation of other genes, cell survival, growth, differentiation, maturation, and functional activity).

Many of the responses to binding of ligands to cell-surface receptors are cytoplasmatic and do not involve immediate gene activation in the nucleus. In some instances, a pre-existing inactive transcription factor following a cell-surface interaction is activated, which leads to immediate gene activation. For example, the protein NF-κB, which can be activated within minutes by a variety of stimuli, including membrane receptors (for example, pattern recognition receptors like Toll-like receptor binding to pathogen-associated molecular patterns), inflammatory cytokines such as TNF-α, IL-1, T-cell activation signals, growth factors and stress inducers.

Our genomic experiment with NMPF peptide LQGV (SEQ ID NO:1) showed very immediate effects on signal transduction and gene regulation since the cells were treated with the peptide for only four hours. In this short period of time, LQGV (SEQ ID NO:1) down-regulated at least 120 genes and up-regulated at least six genes in the presence of a strong stimulator (PHA/IL-2-stimulated T-cell line (PM1)), demonstrating the profound effect on a signaling molecule according to the invention and modulatory effect on gene expression. The genes affected by LQGV (SEQ ID NO:1) include oncogenes, genes for transcription factors, intracellular enzymes, membrane receptors, intracellular receptors, signal-transducing proteins (for example, kinases) and some genes for unknown molecules. This shows that LQGV (SEQ ID NO:1) as an example of the synthetic signaling molecule (oligopeptide or functional analogue or derivative thereof), as described here, has a broad spectrum of effects at different extracellular and intracellular levels. In addition, our HPLC/MS data have shown the presence of LQGV (SEQ ID NO:1) in the nucleus of a macrophage cell line (RAW267.4) within a half hour and also indicates the direct effects on DNA level as well as at an intracellular level, which is further supported by NF-κB experiments. The ultimate modulatory effect of LQGV (SEQ ID NO:1) is dependent on, e.g., type of the cell, differentiation and maturation status of the cell, the functional status and the presence of other regulatory molecules. This was evident by a shock experiment in which different NMPF peptides had similar or different effects on the disease. The same results were obtained with DC, fertilized chicken egg experiments, and CAO experiments; NMPF effects were dependent on type of co-stimulation (GM-CSF alone or in combination with LPS or VEGF) and time of the treatment. Due to this, NMPF have the ability to modulate cellular responses at different levels.

Ischemia induces increased expression of inflammatory cytokines due to activation of NF-κB and AP-1. Inflammatory cytokines can be expressed by endothelium (for example, by trauma), perivascular cells and adherent or transmigrating leukocytes, inducing numerous pro-inflammatory and procoagulant effects. Together these effects predispose to inflammation, thrombosis and hemorrhage. Of clinical and medical interest and value, the present invention provides the opportunity to selectively control NFκB-dependent gene expression in tissues and organs in a living subject, preferably in a primate, allowing up-regulating essentially anti-inflammatory responses such as IL-10, and down-regulating essentially pro-inflammatory responses such as mediated by TNF-α, nitric oxide (NO), IL-5, IL-1β.

The invention thus provides use of an NFκB-regulating peptide or derivative thereof for the production of a pharmaceutical composition for the treatment of an ischemia-reperfusion injury, preferably in a primate, and provides a method of treatment of an ischemia-reperfusion injury, notably in a primate. It is preferred when the treatment comprises administering to the subject a pharmaceutical composition comprising an NF-κB down-regulating peptide or functional analogue thereof. Examples of useful NF-κB down-regulating peptides are VLPALPQVVC (SEQ ID NO:20), LQGVLPALPQ (SEQ ID NO:49), LQG, (SEQ ID NO:2), LQGV (SEQ ID NO:1), GVLPALPQ (SEQ ID NO:33), VLPALP (SEQ ID NO:3), VVC, MTR and circular LQGVLPALPQVVC (SEQ ID NO:34). More down-regulating peptides and functional analogues can be found using the methods as provided herein. Most prominent among NF-κB down-regulating peptides are VLPALPQVVC (SEQ ID NO:20), LQGVLPALPQ (SEQ ID NO:49), LQG, (SEQ ID NO:2), LQGV (SEQ ID NO:1), and VLPALP (SEQ ID NO:3). These are also capable of reducing production of NO by a cell. It is herein also provided to use a composition that comprises at least two oligopeptides or functional analogues thereof, each capable of reducing production of NO and/or TNF-α by a cell, in particular wherein the at least two oligopeptides are selected from the group LQGV (SEQ ID NO:1), (SEQ ID NO:2) and VLPALP (SEQ ID NO:3), for the treatment of an ischemia-reperfusion injury.

In one such instance as provided herein, such a subject has suffered from ischemia-reperfusion injury or has undergone anoxia or infarction. A typical clinical instance is the myocardial infarction or chronic myocardial ischemia of heart tissue in various zones or areas of a living human subject, or, likewise a cerebrovascular infarct, such as a sudden massive infarct of the brain with immediate and possibly grave consequences, but also the so-called silent infarcts that go unnoticed for long times but are thought to be involved in the development of certain forms of dementias.

Typical examples also include other cardiovascular or circulatory disorders, such as heart failure, lacunar brain infarctions, Alzheimer's disease, thrombosis, arteriosclerosis, pregnancy related cardiovascular or circulatory disorders, retinopathies (such as associated with vascular diseases like diabetes) and the like.

In response to a variety of pathophysiological and developmental signals, the NF-κB/Rel family of transcription factors are activated and form different types of hetero- and homodimers among themselves to regulate the expression of target genes containing KB-specific binding sites. NF-κB transcription factors are hetero- or homodimers of a family of related proteins characterized by the Rel homology domain. They form two subfamilies, those containing activation domains (p65-RELA, RELB, and c-REL) and those lacking activation domains (p50, p52). The prototypical NF-κB is a heterodimer of p65 (RELA) and p50 (NF-κB1). Among the activated NF-κB dimers, p50-p65 heterodimers are known to be involved in enhancing the transcription of target genes and p50-p50 homodimers in transcriptional repression. However, p65-p65 homodimers are known for both transcriptional activation and repressive activity against target genes. KB DNA binding sites with varied affinities to different NFB dimers have been discovered in the promoters of several eukaryotic genes and the balance between activated NF-κB homo- and heterodimers ultimately determines the nature and level of gene expression within the cell. The term “NF-κB-regulating peptide” as used herein refers to a peptide or a modification or derivative thereof capable of modulating the activation of members of the NF-κB/Rel family of transcription factors. Activation of NF-κB can lead to enhanced transcription of target genes. Also, it can lead to transcriptional repression of target genes. NF-κB activation can be regulated at multiple levels. For example, the dynamic shuttling of the inactive NF-κB dimers between the cytoplasm and nucleus by IκB proteins and its termination by phosphorylation and proteasomal degradation, direct phosphorylation, acetylation of NF-κB factors, and dynamic reorganization of NF-κB subunits among the activated NF-κB dimers have all been identified as key regulatory steps in NF-κB activation and, consequently, in NF-κB-mediated transcription processes. Thus, an NF-κB-regulating peptide is capable of modulating the transcription of genes that are under the control of NF-κB/Rel family of transcription factors. Modulating comprises the up-regulation or the down-regulation of transcription. In a preferred embodiment, a peptide according to the invention, or a functional derivative or analogue thereof is used for the production of a pharmaceutical composition for the treatment of ischemia-reperfusion injury. Examples of such events are (but not limited to) cerebral vascular accident (CVA), circulatory diseases of the brain, retinopathies (such as associated with vascular diseases like diabetes), circulatory diseases of pregnancy, thrombosis, atherosclerosis, and so on.

An ischemia-reperfusion injury refers to an event in which the blood supply to a tissue is obstructed, such as stroke or myocardial infarction. Due to this obstruction, the endothelial tissue lining the affected blood vessels becomes “sticky” and begins to attract circulating white blood cells. The white cells bound to the endothelium eventually migrate into the brain or cardiac tissue, causing significant tissue destruction. Although neither acute myocardial infarction nor stroke is directly caused by inflammation, much of the underlying pathology and the damage that occurs after an acute ischemia-reperfusion injury is caused by acute inflammatory responses during reperfusion, the restoration of blood flow to the affected organ. Thus, a method is provided herein for treating ischemia-reperfusion injury, including cerebrovascular disease and ischemic heart failure, comprising administering to a subject in need of such a treatment a peptide according to the invention. In particular, a method is provided to control the acute inflammatory response during reperfusion of the affected body part by administering a peptide, or a modification thereof, capable of modulating expression of a gene encoding a pro-inflammatory cytokine. TNF-α is a pro-inflammatory and multifunctional cytokine that has been implicated in diverse pathological processes such as cancer, infection, and autoimmune inflammation. TNF-α has been recently detected in various cardiac-related illnesses including congestive heart failure, myocarditis, dilated and septic cardiomyopathy, and ischemic heart diseases. TNF mRNA and TNF-α protein were detected in explanted hearts from humans with dilated cardiomyopathy and ischemic heart disease, but TNF-α was not detected in nonfailing myocardium. Although the complete portfolio of signaling pathways that are common to both tumor necrosis factor receptor 1 (TNFR1) and tumor necrosis factor receptor 2 (TNFR2) is not known, it is of interest to note that a recently described zinc finger protein, termed tumor necrosis factor receptor associated factor 2 (TRAF2), has been shown to be involved with both TNFR1- and TNFR2-mediated signaling. Consequently, TRAF2-mediated signaling has been shown to activate NF-κB, with a resultant increase in the expression of the antioxidant protein manganese superoxide dismutase (MSOD). Previous studies suggested that the cytoprotective effects of TNF in the setting of myocardial ischemia were mediated through TNF-induced up-regulation of MSOD. It was suggested that pro-inflammatory cytokines such as TNF may play an important role in the timing of cardiac stress response, both by providing early anti-apoptotic cytoprotective signals that are responsible for delimiting cardiac injury and also by providing delayed signals that facilitate tissue repair and remodeling once myocardial damage has supervened. Given the observation that some peptides according to the invention are capable of up-regulating at least one gene in a cell, the invention now provides a method to increase the expression of gene products such as MSOD and other cytoprotective NF-κB-regulated genes. In particular, the invention provides a method for treating an ischemic-reperfusion injury comprising administering to a subject in need of such treatment a signaling molecule comprising a peptide or functional analogue thereof the molecule capable of increasing production of IL-10 by a cell. Increased IL-10 production is for example achieved by treating the subject systemically or treating the subjects infarcted area locally with peptides (SEQ ID NO:2), LQGV (SEQ ID NO:1) or VLPALP (SEQ ID NO:3), or a functional analogue thereof similarly capable of modulating translocation and/or activity of a gene transcription factor present in a cell in the ischemic or infarcted area. These peptides have the added advantage that TNF-α production by the cell is reduced. When taking ischemic heart failure as an example, an NF-κB down-regulating peptide according to the invention can, for example, be introduced locally to the infarcted area directly as a synthesized compound to living cells and tissues via a range of different delivery means. These include the following.

1. Intracoronary delivery is accomplished using catheter-based deliveries of synthesized peptide (or derivative) suspended in a suitable buffer (such as saline) which can be injected locally (i.e., by injecting into the myocardium through the vessel wall) in the coronary artery using a suitable local delivery catheter such as a 10 mm InfusaSleeve catheter (Local Med, Palo Alto, Calif.) loaded over a 3.0 mm×20 mm angioplasty balloon, delivered over a 0.014 inch angioplasty guidewire. Delivery is typically accomplished by first inflating the angioplasty balloon to 30 psi, and then delivering the protein through the local delivery catheter at 80 psi over 30 seconds (this can be modified to suit the delivery catheter).

2. Intracoronary bolus infusion of peptide (or derivative) synthesized previously can be accomplished by a manual injection of the substance through an Ultrafuse-X dual lumen catheter (SciMed, Minneapolis, Minn.) or another suitable device into proximal orifices of coronary arteries over 10 minutes.

3. Pericardial delivery of synthesized peptide (or derivative) is typically accomplished by installation of the peptide-containing solution into the pericardial sac. The pericardium is accessed via a right atrial puncture, transthoracic puncture or via a direct surgical approach. Once the access is established, the peptide material is infused into the pericardial cavity and the catheter is withdrawn. Alternatively, the delivery is accomplished via the aid of slow-release polymers such as heparinal-alginate or ethylene vinyl acetate (EVAc). In both cases, once the peptide (or derivative) is integrated into the polymer, the desired amount of peptide/polymer is inserted under the epicardial fat or secured to the myocardial surface using, for example, sutures. In addition, the peptide/polymer composition can be positioned along the adventitial surface of coronary vessels.

4. Intramyocardial delivery of synthesized peptide (or derivative) can be accomplished either under direct vision following thoracotomy or using thoracoscope or via a catheter. In either case, the peptide containing solution is injected using a syringe or other suitable device directly into the myocardium.

Up to 2 cc of volume can be injected into any given spot and multiple locations (up to 30 injections) can be done in each patient. Catheter-based injections are carried out under fluoroscopic, ultrasound or Biosense NOGA guidance. In all cases after catheter introduction into the left ventricle the desired area of the myocardium is injected using a catheter that allows for controlled local delivery of the material. Of course, similar techniques are applied to administer the peptide locally to other infarcted areas, such as seen with cerebrovascular incidents.

Means and methods for making the oligopeptide, acid and base salts, etc. are described in U.S. Patent Application Publication 20060142205 A1 to Benner et al. (Jun. 29, 2006), the contents of which are incorporated herein by this reference.

In a further embodiment, the invention provides a method for modulating a cerebral ischemia-reperfusion injury in a subject comprising providing the subject with a signaling molecule comprising a gene-regulatory peptide or functional analogue thereof in combination therapy with thrombolysis. Two major strategies can be used to reduce the neuronal damage following cerebral ischemia: restoration of cerebral blood perfusion through usage of thrombolytics and inhibition of the apoptotic and inflammatory cascades which result from ischemia through usage of a peptide or functional analogue according to the invention. Combining both treatment strategies provides additional benefits to those achieved by using the individual strategies alone. For instance, restoration of blood flow improves perfusion of the ischemic brain tissue with peptide compositions and enhances their protective effects. Thrombolysis and/or prevention of thrombi is, for example, achieved by intravenous injection of heparin, in a bolus of 5,000 IU followed by infusion of 15,000 units/hour to induce an APTT-ratio of 2.0. Alternatively; intramuscular injections of low-molecular weight heparin, such as fragmin of 200 IU/kg/day in 2 daily doses, are given. Intra-arterial thrombolysis is preferably applied within 3 hours of onset of ischemic stroke. In short, selective intra-arterial digital substraction angiography is performed on a biplane, high-resolution angiography system (for example a Toshiba CAS 500) with a matrix of 1024×1024 pixels. A 5.5.F-JB2 catheter (Valavanis) is inserted in the femoral artery and guided to the cerebral arteries for diagnostic 4-vessel angiography. A microcatheter, mostly a Fast Tracker 18 (Target Therapeutics) through the 5.5-F JB2 catheter is navigated into the cerebral arteria corresponding with the ischemic brain area. A microcatheter is navigated into the occluded cerebral artery. Urokinase (Urokinase HS Medac) in a mean dose usually ranging from 20,000 to 1,250,000 IU is infused directly into or near the proximal end of the occluding thrombus over 60 to 90 minutes. For mechanical disruption and removal of the thrombotic material additional usage of a very flexible hydrophilic guide wire catheter with a J shape tip to avoid perforation of the vessel wall (for example a Silver Speed MTI 0.008 or 0.010 inch) may be necessary. In addition to agents for thrombylosis and/or prevention of thrombosis, whether applied intravenously, intramuscularly or intra-arterial, treatment with peptide composition is preferably started at the same time. The invention also provides a method for modulating an ischemia-reperfusion injury in a subject comprising providing the subject with a signaling molecule comprising a gene-regulatory peptide or functional analogue thereof for the prevention of cerebral ischemia in patients with defined at risk periods. Some conditions are frequently followed by cerebral ischemia, in which a peptide or functional analogue thereof is valuable to prevent infarction, illustrated in two specific examples.

(1) Cardiac or aortic surgery is frequently complicated with severe hypotensive periods and/or thrombo-embolic events which may result in cerebral or myelum ischemia and infarction. The peptide composition according to the invention can be given in all or a specific subgroup of these patients, before, during and/or after surgery to prevent cerebral ischemia.

(2) The final outcome in patients with aneurysmatic subarachnoid hemorrhagia (SAH) is largely determined by the development of cerebral ischemia in the subsequent 3 weeks. SAH is a life threatening intracerebral bleeding, usually due to a rupture of an aneurysm of the cerebral arteries in the circulus Willisi. SAH affects 10.5 per 100,000 persons per year of which one-third will die. Up to one-third of patients will develop cerebral ischemia in the 3 weeks after SAH, which determines the final outcome and for which all patients with SAH will be admitted to intensive care units. The pathophysiology of cerebral ischemia after SAH is not precisely known, but a specific role is claimed for the presence of subarachnoid blood and/or intracerebral inflammation and vasospasms. Treatment to prevent cerebral ischemia, including triple H-therapy (hypervolemia, hemodilution, hypertension), vasodilators, and endovascular approaches to symptomatic vasospasms, thus far are insufficient in many patients. An NF-κB down-regulating peptide should be given in this 3 weeks following SAH, alone or in combination with other forms of preventive treatments, during which these patients are at risk to develop cerebral ischemia and can be monitored at the intensive care unit.

In these two and other conditions in which there is a limited period with a significant increase to develop cerebral ischemia, an NF-κB down-regulating peptide can be used to prevent (further) cerebral ischemia and improve final clinical outcome.

The invention furthermore provides a method to monitor and titrate therapeutic effect of a treatment with peptide according to the invention in patients with cerebral ischemia. This is foremost achieved by clinical evaluation according to predefined neurological deficit-, disability- and handicap scales, such as the Oxford-handicap scale. CT, CT-angiography, MRI, MR-angiography, and SPECT-scan can be done. Also, cytokines, soluble cytokine-receptors, chemokines are determined in follow-up plasma and cerebrospinal fluid (CSF) samples. Follow-up CSF samples can be obtained by permanent monitoring via ventricular catheters. Intracerebral HPLC sensors provide for determining parenchyma oxygen, pH and small metabolites including lactate, pyruvate and glucose. This device is already in use in combination with intracranial pressure bolds to monitor the cerebral parenchyma of patients with contusio cerebri.

Preferred routes of administration of a peptide or functional analogue thereof according to the invention in patients with cerebral ischemia are:

Intravenously in 0.9% saline solutions according to protocol.

Intrathecally. In short, the peptide composition may be given after a lumbar puncture with a 18 G needle or after subsequent insertion of a extralumbal catheter with the tip in the intrathecal space. This way of drug administration can not be used in patients with large infarctions and danger of replacement of brain tissue or herniation, but is a useful way in treating patients with a SAH. Intrathecal drug administration is an established route of drug administration in patients with leukemia and multiple sclerosis. In patients with SAH extralumbar drains are already frequently used to prevent or treat hydrocephalus, a common complication in SAH.

Intra-arterial. A similar protocol is used as in intra-arterial thrombolysis. In short, selective intra-arterial digital substraction angiography is performed on a biplane, high-resolution angiography system (for example a Toshiba CAS 500) with a matrix of 1024×1024 pixels. A 5.5.F-JB2 catheter (Valavanis) is inserted in the femoral artery and guided to the cerebral arteries for diagnostic 4-vessel angiography. A microcatheter, mostly a Fast Tracker 18 (Target Therapeutics) through the 5.5-F JB2 catheter is navigated into the cerebral arteria corresponding with the ischemic brain area. Perfusion of this area with peptide is achieved according to this protocol. This route of administration is of special interest in case of combination therapy with intra-arterial thrombolysis. In that case, the same devices and protocols are used in which the microcatheter is navigated into the occluded cerebral artery. Urokinase (Urokinase HS Medac) in a mean dose usually ranging from 20,000 to 1,250,000 is infused directly into or near the proximal end of the occluding thrombus over 60 to 90 minutes. For mechanical disruption and removal of the thrombotic material additional usage of a very flexible hydrophilic guide wire catheter with a J shape tip to avoid perforation of the vessel wall (for example a Silver Speed MTI 0.008 or 0.010 inch) may be necessary. Furthermore, a peptide or functional analogue may be applied locally after craniotomy. A range of suitable pharmaceutical carriers and vehicles are known conventionally to those skilled in the art. Thus, for parenteral or systemic administration, the peptide compound will typically be dissolved or suspended in sterile water or saline. Typically, systemic administration involves intravenous administration, for example per infusionem. Especially when the subject is at risk to experience iatrogenic reperfusion injury occurring after the ischemia-reperfusion injury, for example due to treatment with an anticoagulant or a thrombolytic agent, systemic administration per infusionem is advantageous, as the risk of bleeding is increased in such patients, necessitating the reduction of invasive measures such as the use of catheters or other puncturing techniques.

Improvement in neurological diseases is limited due to the restricted regeneration capacity of neurons, especially in the central nervous system (CNS). For this reason, and for the high susceptibility of neurons to ischemia and inflammation, treatment strategies in neurology more than in other medical disciplines, focus on an immediate prevention of (further) neural damage. Ischemia and inflammation of neural tissue are mediated by similar pathogenic pathways leading to and mediated by release and activation of transcription factors, such as NF-κB, and cytokines, such as TNFα. In addition, in many neurological diseases ischemic and inflammatory processes both contribute to (further) tissue damage. More than other diseases, neurological disorders will, therefore, profit from immune-mediating agents, such as by treatment with an β-hCG oligopeptide derivate such as an NF-κB down-regulating peptide according to the invention, that has an immediate and pleiotropic effect and inhibit these common pathways in both ischemic and inflammatory processes. The invention also provides a method for treating cerebral infarction with an NF-κB down-regulating peptide according to the invention.

Cerebral infarction is a common and disabling neurological disease which results from an acute onset insufficient arterial blood supply and ischemia of the associated territorial brain. The causes of the acute insufficient perfusion are (1) thrombo-embolic events related to atherosclerosis of large cerebral arteria and/or cardiac diseases leading to cortical infarctions, (2) hypotension leading to so called “watershed infarctions,” and (3) small vessel diseases related to hypertension and atherosclerosis leading to lacunar infarctions. Each type of infarction may induce distinct patterns of neurological deficits related to the function of the damaged brain area. All these types of infarctions, especially multiple lacunar infarctions, may contribute to the development of vascular (or post-stroke) dementia.

Neurological deficits in stroke are potentially reversible, provided the duration of ischemia is short, such as in “transient ischemic attacks” (TIAs). Partial spontaneous improvement in ischemic strokes most likely results from reversible dysfunction of the penumbrae area, where ischemia does not evolve into infarction. The invention also provides treatment of ischemic stroke patients with thrombolytic agents combined with treatment with an NF-κB down-regulating peptide according to the invention, preferably within 3 hours after onset of neurological symptoms when cerebral ischemia in potential is a treatable state.

Permanent neurological deficit in stroke patients is due to apoptotic cell death of infarcted brain tissue caused by long lasting ischemic periods and subsequent activation of apoptotic pathways during the reperfusion phase. Ischemia induces depolarization and release of excitatory amino acids such as glutamate leading to Ca²⁺ and water influx, which successively leads to cerebral edema and Ca²⁺-mediated inflammatory and degenerative processes. Ischemia induces increased expression of TNF and activation of NF-κB. TNF can be expressed by endothelium (for example, by trauma), perivascular cells and adherent or transmigrating leukocytes, inducing numerous pro-inflammatory and procoagulant effects. Together these effects predispose to local inflammation, thrombosis and hemorrhage. As such they can contribute to stroke initiation, progression of brain damage and in development of tolerance to ischemia. In addition, TNF may contribute to repair and recovery after stroke as an important mediator and modulator of inflammation. β-hCG oligopeptide derivates are known to inhibit TNF expression and NF-κB activation and successive inflammatory and apoptotic pathways. These characteristics should enable single β-hCG oligopeptide derivates or cocktails of derivates to prevent the further brain ischemia and infarction and the occurrence of complications, including cerebral edema and secondary hemorrhages, which may contribute to improvement of clinical outcome in stroke patients. Ischemia and infarction secondary to cerebral contusion and to epi-, subarachnoid- and subdural hemorrhages play a significant role in final brain damage and clinical outcome in patients with these disorders. Local TNF expression and NF-κB activation due to ischemia in these diseases will predispose to local inflammation, thrombosis and hemorrhage, similar to ischemic stroke patients. Therefore administration of single NF-κB down-regulating peptides or mixtures thereof contributes to improvement of final outcome also in these diseases. In patients with contusio cerebri and intracranial pressure treatment it is advantageous to combine treatment with the peptides or functional analogues thereof with osmotic agents like mannitol to reduce intracranial pressure and stimulate cerebral perfusion, i.e., by administering intravenous infusions of mannitol 20% in 0.9% saline solutions of 200 ml, or another hypertonic solution, 1 to 6 times a day. NF-κB-regulating peptide can be given in the same infusion, the peptide (or analogue) concentration preferably being from about 1 to about 1000 mg/l, but the peptide can also be given in a bolus injection. Doses of 1 to 5 mg/kg bodyweight, for example every eight hours in a bolus injection or per infusionem until the patient stabilizes, are recommended. For example in cases where large infarcted areas are expected or diagnosed, it is preferred to monitor cytokine profiles, such as TNF-α or IL-10 levels, in the plasma (or cerebrospinal fluid) of the treated patient, and to stop treatment when these levels are normal. In patients with contusio cerebri, intracranial pressure and intraparechymal oxygen and metabolites can be monitored using intracranial sensors. In a preferred embodiment, the invention provides a method of treating a subject suffering from an ischemia-reperfusion injury with a method and signaling molecule according to the invention concomitantly, or at least timely, with a thrombolytic agent, such as (recombinant) tissue plasminogen activator, or truncated forms thereof having tissue plasminogen activity, or streptokinase, or urokinase. In the case of a cerebrovascular incident, such treatment can, for example, take the form of intravenous infusions of recombinant tissue plasminogen activator (rt-PA) at a dose of 0.9 mg/kg (maximum of 90 mg) in 0.9% saline solutions, whereby it is preferred that 10% of the rt-PA dose is given within 1 to 2 minutes and the remaining dose of rt-PA in 60 minutes. In the case of an acute myocardial infarction, such treatment can for example take the form of intravenous infusions of rt-PA at a dose of 15 mg as intravenous bolus, followed by 50 mg in the next 30 minutes followed by 35 mg in the next 60 minutes. For the sake of treating the resulting perfusion injury that occurs due to the lysis of the thrombus and the subsequent perfusion of the ischemic area, it is herein provided to also provide the patient with a bolus injection of NF-κ down-regulating peptide such as (SEQ ID NO:2), LQGV (SEQ ID NO:1) or VLPALP (SEQ ID NO:3) at 2 mg/kg and continue the infusion with an NF-κB down-regulating peptide such as (SEQ ID NO:2), LQGV (SEQ ID NO:1) or VLPALP (SEQ ID NO:3) or a functional analogue thereof at a dose of 1 mg/kg bodyweight for every eight hours. Dosages may be increased or decreased, for example depending on the outcome of monitoring the cytokine profile in the plasma of the patient. In one embodiment of the present invention, a signal molecule is administered in an effective concentration to an animal or human systemically, e.g., by intravenous, intra-muscular or intraperitoneal administration. Another way of administration comprises perfusion of organs or tissue, be it in vivo or ex vivo, with a perfusion fluid comprising a signal molecule according to the invention. Topical administration, e.g., in ointments or sprays, may also apply, e.g., in or around infarcted areas in brain or heart, etc. The administration may be done as a single dose, as a discontinuous sequence of various doses, or continuously for a period of time sufficient to permit substantial modulation of gene expression. In the case of a continuous administration, the duration of the administration may vary depending upon a number of factors which would readily be appreciated by those skilled in the art.

The administration dose of the active molecule may be varied over a fairly broad range. The concentrations of an active molecule which can be administered would be limited by efficacy at the lower end and the solubility of the compound at the upper end. The optimal dose or doses for a particular patient should and can be determined by the physician or medical specialist involved, taking into consideration well-known relevant factors such as the condition, weight and age of the patient, etc.

Ten male C57BL/6 mice (23 to 26 g), 5 controls and 5 test animals were used as a model for ischemic stroke by middle cerebral artery occlusion/reperfusion. These mice are initially anesthetized with metofane and maintained with i.p. ketamine (60 mg/ml) and xylazine (5 mg/ml). Atropine methyl nitrate (0.18 mg/kg i.p.) is given to prevent airway obstruction. Animals are allowed to breathe spontaneously. A modified intravascular middle cerebral artery (MCA) occlusion technique is used to induce stroke. A nonsiliconized uncoated 6-0, 8-mm-long prolene suture with a rounded tip (diameter 0.20 mm) is advanced into the internal carotid artery to occlude the MCA for 1 hour, followed by 24 hours of reperfusion.

Cerebral blood perfusion (CBF) is monitored by laser Doppler flowmetry (Transonic Systems). Laser Doppler flowmetry probes (0.8 mm in diameter) are positioned on the cortical surface 2 mm posterior to the bregma, both 3 and 6 mm to each side of midline. The procedure is considered to be successful if a >85% drop in CBF was observed immediately after placement of the suture.

Survival and neurological deficits is monitored and scored as follows: no neurological deficit (0), failure to extend forepaw fully (1), turning to left (2), circling to left (3), unable to walk spontaneously (4), stroke-related death (5).

Arterial blood gases (pH, PaO2, PaCO2) are measured before and during MCA occlusion with an ABL 30 Acid-Base Analyzer (Radiometer).

### Reducing Cerebral Ischemia by Gene-Regulatory Peptides in Murine Model

Each of the 5 test mice received a 1:1 mixture of LQGV (SEQ ID NO:1) and VLPALP (SEQ ID NO:3) at 5 mg/kg in a volume of 0.5 ml 0.9% saline which was given intravenously at 10 minutes, when blood flow was at a minimum, since these conditions would give a reasonable test of the bioactivity of these peptides during ischemia, because the time course of pathophysiological changes in the present murine model is different from that of human strokes, and the occlusion is experimentally removed after 1 hour. Each of the control mice received 0.5 ml 0.9% saline i.v.

Administration of peptides after 2 to 3 hours in this murine model would actually be during the reperfusion phase, which may not be fully relevant to the human clinical situation where treatment would be desired within 1 hour after the stroke, because complete reopening of major occluded blood vessels in humans who experience ischemic stroke might not typically happen spontaneously 1 hour after the onset of ischemic stroke.

Early results control mice scored: 3, 5, 4, 2, 4; test mice scored: 3, 1, 3, 2, 1

Possible mechanisms by which peptides reduce cerebral ischemia in murine model. Not wishing to be bound by theory, the thrombomodulin-protein C (TM-PC) pathway is known to function on endothelium and to counterbalance coagulation. In addition the TM-PC pathway provides protective signaling that counteracts apoptosis in response to oxygen deprivation. Activated protein C (APC) is a systemic anti-coagulant and anti-inflammatory factor which has been demonstrated to protect the brain from ischemic injury. Cytoprotection of brain endothelium by APC in vitro required endothelial protein C receptor (EPCR) and protease-activated receptor-1 (PAR-1), as did in vivo neuroprotective activity in the murine stroke model. It reduces organ damage in animal models of sepsis, ischemic injury and stroke. This invention shows that the above used gene-regulatory peptides reduce inflammatory mediators, activation of transcription factors, including NF-κB, and directly interfere with the TM-PC pathway. The neuroprotection induced by these peptides is mediated by one or combinations of these effects. Peptides in this way act as a direct cell survival factor and reduce secondary ischemia by their anti-coagulant and anti-inflammatory effects.

### EXAMPLES

### Material and Methods

### Peptide Synthesis

The peptides as mentioned in this document such as LQG, AQG, LQGV (SEQ ID NO:1), (SEQ ID NO:2), LQGA (SEQ ID NO:19), VLPALP (SEQ ID NO:3), ALPALP (SEQ ID NO:21), VAPALP (SEQ ID NO:22), ALPALPQ (SEQ ID NO:23), VLPAAPQ (SEQ ID NO:24), VLPALAQ (SEQ ID NO:25), LAGV (SEQ ID NO:26), VLAALP (SEQ ID NO:27), VLPALA (SEQ ID NO:28), VLPALPQ (SEQ ID NO:29), VLAALPQ (SEQ ID NO:30), VLPALPA (SEQ ID NO:31), GVLPALP (SEQ ID NO:32), VVCNYRDVRFESIRLPGCPRGVNPVVSYAVAL SCQCAL (SEQ ID NO:35), RPRCRPINATLAVEKEGCPVCITVNTTICAGYCPT (SEQ ID NO:45), SKAPPPSLPSPSRLPGPS (SEQ ID NO:38), LQGVLPALPQVVC (SEQ ID NO:34), SIRLPGCPRGVNPVVS (SEQ ID NO:39), LPGCPRGVNPVVS (SEQ ID NO:40), LPGC (SEQ ID NO:41), MTRV (SEQ ID NO:42), MTR, and VVC were prepared by solid-phase synthesis (Merrifield, 1963) using the fluorenylmethoxycarbonyl (Fmoc)/tert-butyl-based methodology (Atherton, 1985) with 2-chlorotrityl chloride resin (Barlos, 1991) as the solid support. The side-chain of glutamine was protected with a trityl function. The peptides were synthesized manually. Each coupling consisted of the following steps: (i) removal of the alpha-amino Fmoc-protection by piperidine in dimethylformamide (DMF), (ii) coupling of the Fmoc amino acid (3 eq) with diisopropylcarbodiimide (DIC)/1-hydroxybenzotriazole (HOBt) in DMF/N-methylformamide (NMP), and (iii) capping of the remaining amino functions with acetic anhydride/diisopropylethylamine (DIEA) in DMF/NMP. Upon completion of the synthesis, the peptide resin was treated with a mixture of trifluoroacetic acid (TFA)/H₂O/triisopropylsilane (TIS) 95:2.5:2.5. After 30 minutes, TIS was added until decoloration. The solution was evaporated in vacuo and the peptide precipitated with diethylether. The crude peptides were dissolved in water (50 to 100 mg/ml) and purified by reverse-phase high-performance liquid chromatography (RP-HPLC). HPLC conditions were: column: Vydac TP21810C18 (10×250 mm); elution system: gradient system of 0.1% TFA in water v/v (A) and 0.1% TFA in acetonitrile (ACN) v/v (B); flow rate 6 ml/min; absorbance was detected from 190 to 370 nm. There were different gradient systems used. For example, for peptides LQG and LQGV (SEQ ID NO:1): ten minutes 100% A followed by linear gradient 0 to 10% B in 50 minutes. For example, for peptides VLPALP (SEQ ID NO:3) and VLPALPQ (SEQ ID NO:29): five minutes 5% B followed by linear gradient 1% B/minute. The collected fractions were concentrated to about 5 ml by rotation film evaporation under reduced pressure at 40° C. The remaining TFA was exchanged against acetate by eluting two times over a column with anion exchange resin (Merck II) in acetate form. The elute was concentrated and lyophilized in 28 hours. Peptides later were prepared for use by dissolving them in PBS.

### Transcription Factor Experiment

Macrophage cell line. The RAW 264.7 macrophages, obtained from American Type Culture Collection (ATCC number TIB-71; Manassas, Va.), were cultured at 37° C. in 5% CO₂ using DMEM containing 10% FBS and antibiotics (100 U/ml of penicillin and 100 μg/ml streptomycin). Cells (1×10⁶/ml) were incubated with peptide (10 μg/ml) in a volume of 2 ml. After eight hours of cultures, cells were washed and prepared for nuclear extracts.

Nuclear extracts. Nuclear extracts and EMSA were prepared according to Schreiber et al. methods (Schriber et al. 1989, Nucleic Acids Research 17). Briefly, nuclear extracts from peptide-stimulated or nonstimulated macrophages were prepared by cell lysis followed by nuclear lysis. Cells were then suspended in 400 μl of buffer (10 mM HEPES (pH 7.9), 10 mM KCl, 0.1 mM KCL, 0.1 mM EDTA, 0.1 mM EGTA, 1 mM DTT, 0.5 mM PMSF and protease inhibitors), vigorously vortexed for 15 seconds, left standing at 4° C. for 15 minutes, and centrifuged at 15,000 rpm for two minutes. The pelleted nuclei were resuspended in buffer (20 mM HEPES (pH 7.9), 10% glycerol, 400 mM NaCl, 1 mM EDTA, 1 mM EGTA, 1 mM DTT, 0.5 mM PMSF and protease inhibitors) for 30 minutes on ice, then the lysates were centrifuged at 15,000 rpm for two minutes. The supernatants containing the solubilized nuclear proteins were stored at −70° C. until used for the Electrophoretic Mobility Shift Assays (EMSA).

EMSA. Electrophoretic mobility shift assays were performed by incubating nuclear extracts prepared from control (RAW 264.7) and peptide-treated RAW 264.7 cells with a 32P-labeled double-stranded probe (5′ AGCTCAGAGGGGGACTTTCCGAGAG 3′ (SEQ ID NO:51)) synthesized to represent the NF-κB binding sequence. Shortly, the probe was end-labeled with T4 polynucleotide kinase according to manufacturer's instructions (Promega, Madison, Wis.). The annealed probe was incubated with nuclear extract as follows: in EMSA, binding reaction mixtures (20 μl) contained 0.25 μg of poly(dI-dC) (Amersham Pharmacia Biotech) and 20,000 rpm of 32P-labeled DNA probe in binding buffer consisting of 5 mM EDTA, 20% Ficoll, 5 mM DTT, 300 mM KCl and 50 mM HEPES. The binding reaction was started by the addition of cell extracts (10 μg) and was continued for 30 minutes at room temperature. The DNA-protein complex was resolved from free oligonucleotide by electrophoresis in a 6% polyacrylamide gel. The gels were dried and exposed to x-ray films.

### ApoE Experiments

Apolipoprotein E (apoE) deficiency is associated with a series of pathological conditions including dyslipidemia, atherosclerosis, Alzheimer's disease, increased body weight and shorter life span. Inheritance of different alleles of the polymorphic apoE gene is responsible for 10% of the variation in plasma cholesterol in most populations. Individuals homozygous for one variant, apoE2, can develop type III dysbetalipoproteinaemia if an additional genetic or environmental factor is present. Some much rarer alleles of apoE produce dominant expression of this disorder in heterozygous individuals. ApoE is a ligand for the LDL receptor and its effects on plasma cholesterol are mediated by differences in the affinity of the LDL receptor for lipoproteins carrying variant apoE proteins. The factors that regulate apoE gene transcription have been investigated extensively by the expression of gene constructs in transgenic mice and involve complex interactions between factors that bind elements in the 5′ promoter region, in the first intron and in 3′ regions many kilobases distant from the structural gene. Deletion of the apoE gene is associated with changes in lipoprotein metabolism (plasma total cholesterol), HDL cholesterol, HDL/TC, and HDL/LDL ratios, esterification rate in apo B-depleted plasma, plasma triglyceride, hepatic HMG-CoA reductase activity, hepatic cholesterol content, decreased plasma homocyst(e)ine and glucose levels, and severe atherosclerosis and cutaneous xanthomatosis.

### Results

### NF-κB Experiments

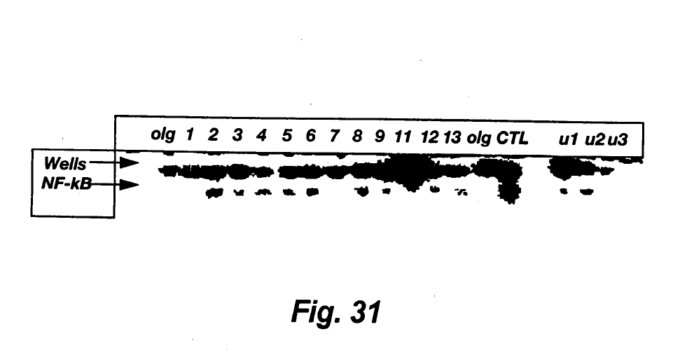

The transcription factor NF-κB participates in the transcriptional regulation of a variety of genes. Nuclear protein extracts were prepared from LPS- and peptide-treated RAW264.7 cells or from LPS-treated RAW264.7 cells. In order to determine whether the peptide modulates the translocation of NF-κB into the nucleus, on these extracts EMSA was performed. FIG. 31 shows the amount of NF-κB present in the nuclear extracts of RAW264.7 cells treated with LPS or LPS in combination with peptide for four hours. Here we see that indeed some peptides are able to modulate the translocation of NF-κB since the amount of labeled oligonucleotide for NF-κB is reduced. In this experiment, peptides that show the modulation of translocation of NF-κB are: (NMPF-1)VLPALPQVVC (SEQ ID NO:20), (NMPF-2)LQGVLPALPQ (SEQ ID NO:49), (NMPF-3)LQG, (NMPF-4)LQGV (SEQ ID NO:1), (NMPF-5)GVLPALPQ (SEQ ID NO:33), (NMPF-6)VLPALP (SEQ ID NO:3), (NMPF-7)VLPALPQ (SEQ ID NO:29), (NMPF-8)GVLPALP (SEQ ID NO:32), (NMPF-9)VVC, (NMPF-11)MTRV (SEQ ID NO:42), (NMPF-12)MTR.

### Nuclear Location of Peptide Experiment

A reverse-phase high-performance liquid chromatography (RP-HPLC) method was used to prove the presence of synthetic oligopeptide in the nuclear extracts. We used a Shimadzu HPLC system equipped with a Vydac monomeric C18 column (column 218MS54, LC/MS C18 reversed phase, 300 A, 5 μm, 4.6 mm ID×250 mm L); elution system: gradient system of 0.01% TFA and 5% acetonitrile (CAN) in water v/v (A) and 0.01% TFA in 80% acetonitrile (ACN) v/v (B); flow rate 0.5 ml/min; absorbance was detected from 190 to 370 nm. The gradient time program was as follows:

| Time (minutes) | Buffer B concentration |
|---|---|
| 0.01 | 0 |
| 5.0 | 0 |
| 30.0 | 80 |
| 40.0 | 100 |
| 60.0 | 100 |
| 65.0 | 0 |
| 70.0 | 0 |

The elution time of peptide LQGV (SEQ ID NO:1) was determined by injecting 2 μg of the peptide in a separate run. Mass spectrometry (MS) analysis of fraction which contained possible NMPF-4 (LQGV (SEQ ID NO:1)) (elution time was determined by injecting the peptide in the same or separate run) was performed on LCQ Deca XP (Thermo Finnigan).

### Results

### Nuclear Location of Peptide Experiment

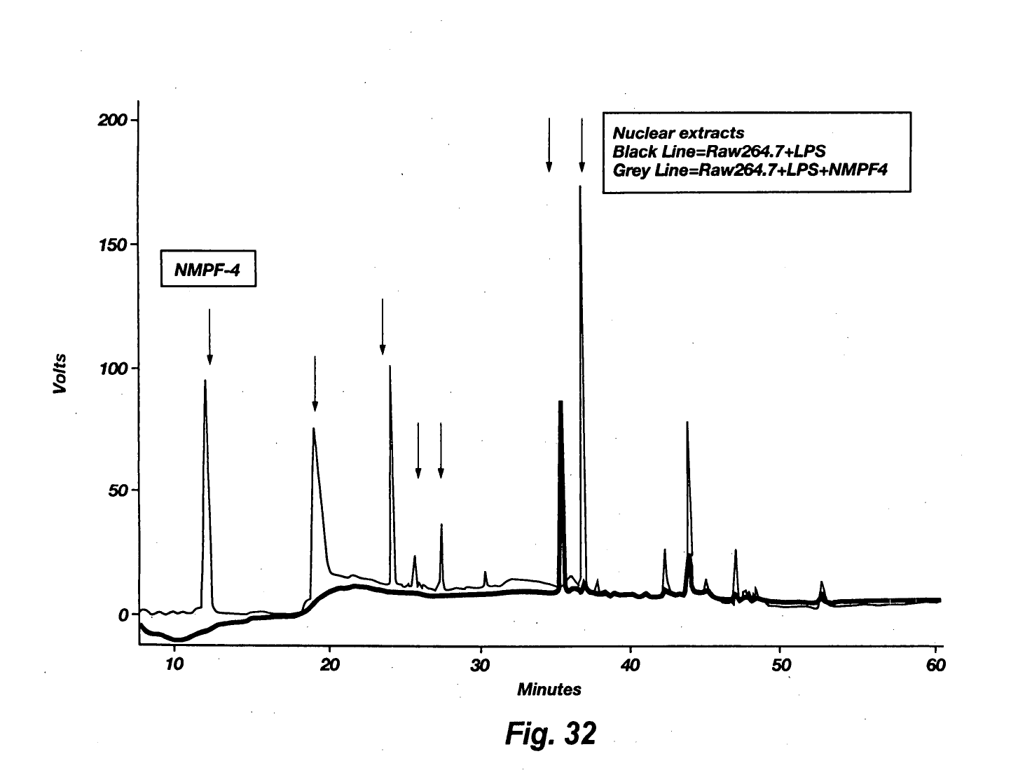

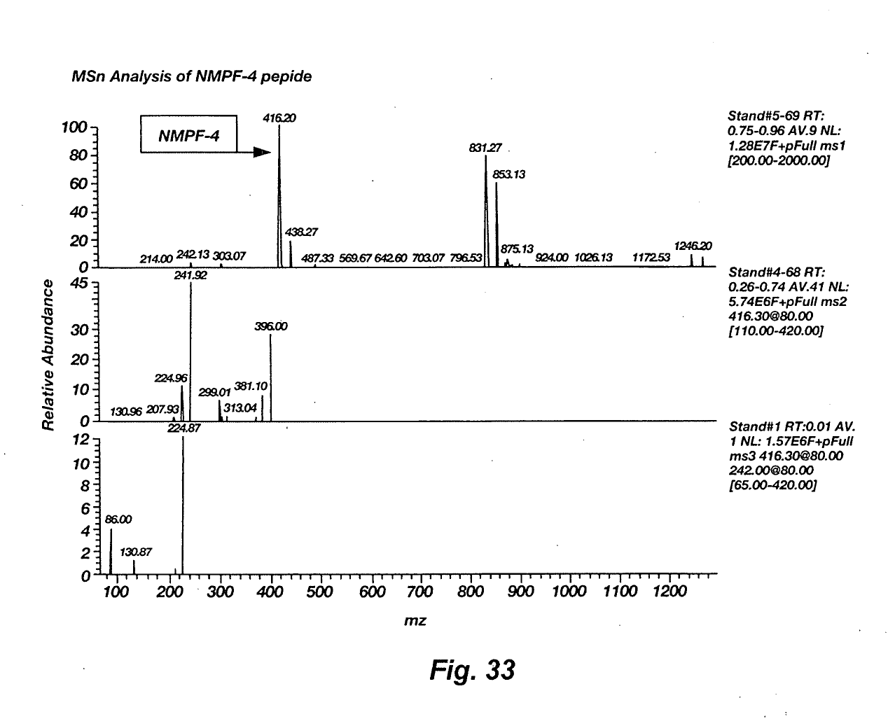

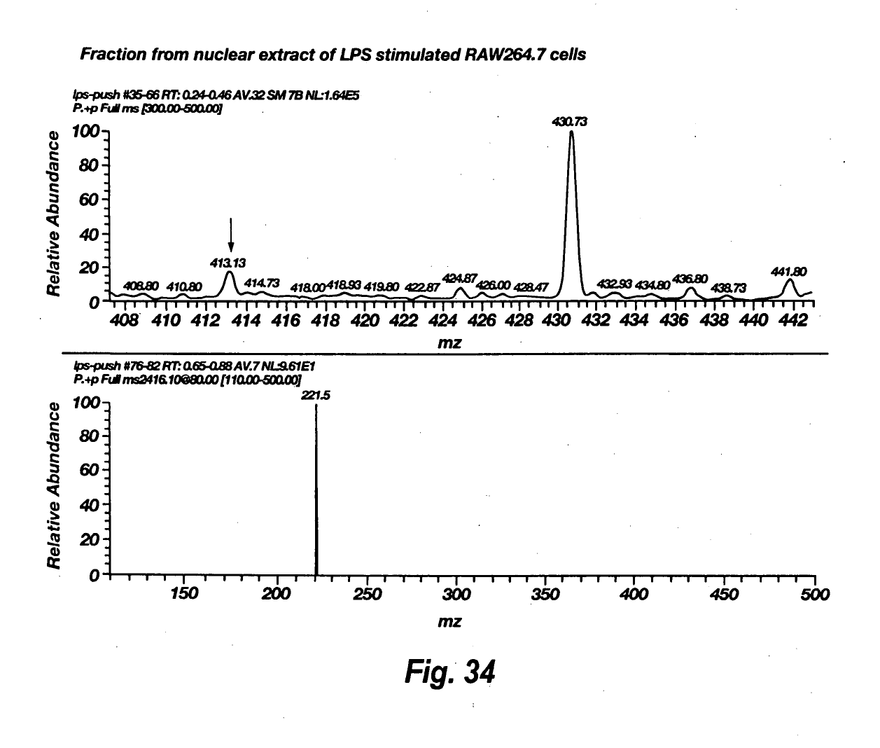

The nuclear protein extracts used in EMSA experiments were also checked for the presence of LQGV (SEQ ID NO:1) by means of HPLC and MS. FIG. 32 shows HPLC chromatograph (wavelength 206) in which data profiles obtained from the nuclear protein extracts of LPS and LPS in combination with NMPF-4 (LQGV (SEQ ID NO:1))-stimulated RAW264.7 cells are overlayed. This figure also shows the presence or absence of number of molecule signals in the nuclear extracts of oligopeptide+LPS-treated cells as compared to nuclear extracts of LPS-treated cells. Since the HPLC profile of LQGV (SEQ ID NO:1) showed that the peptide elutes at around 12 minutes (data not shown), fraction corresponding to region 10 to 15 minutes was collected and analyzed for the presence of this peptide in MS.

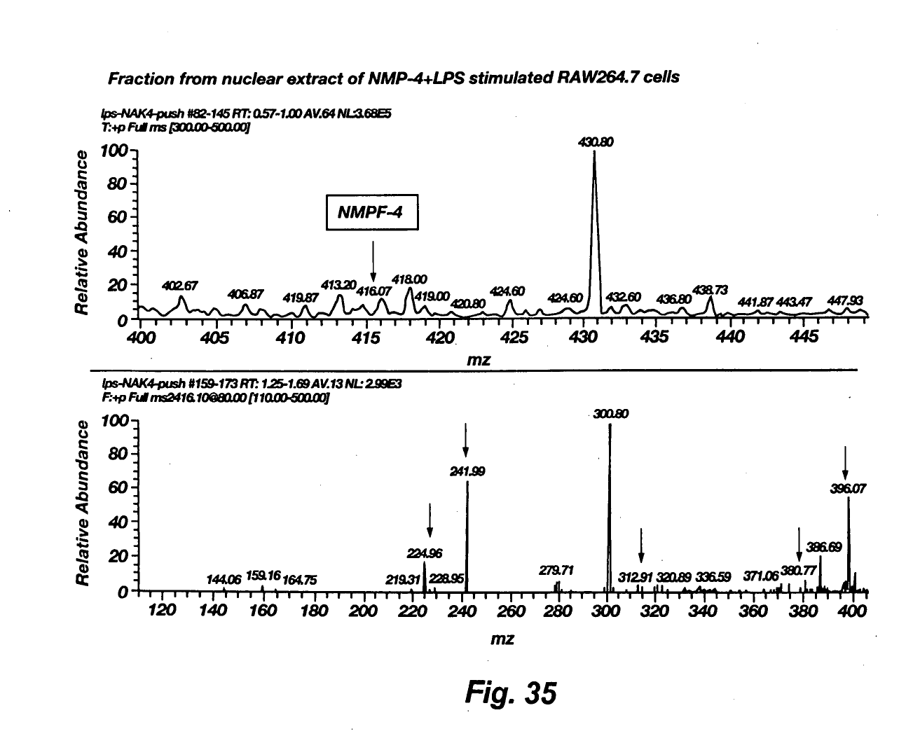

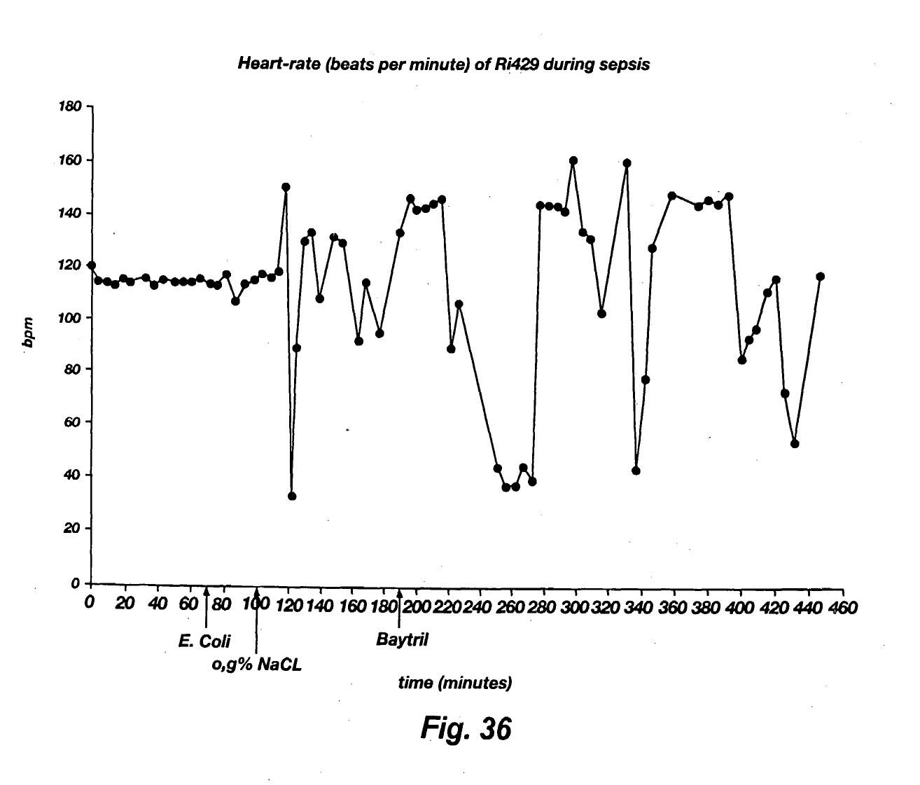

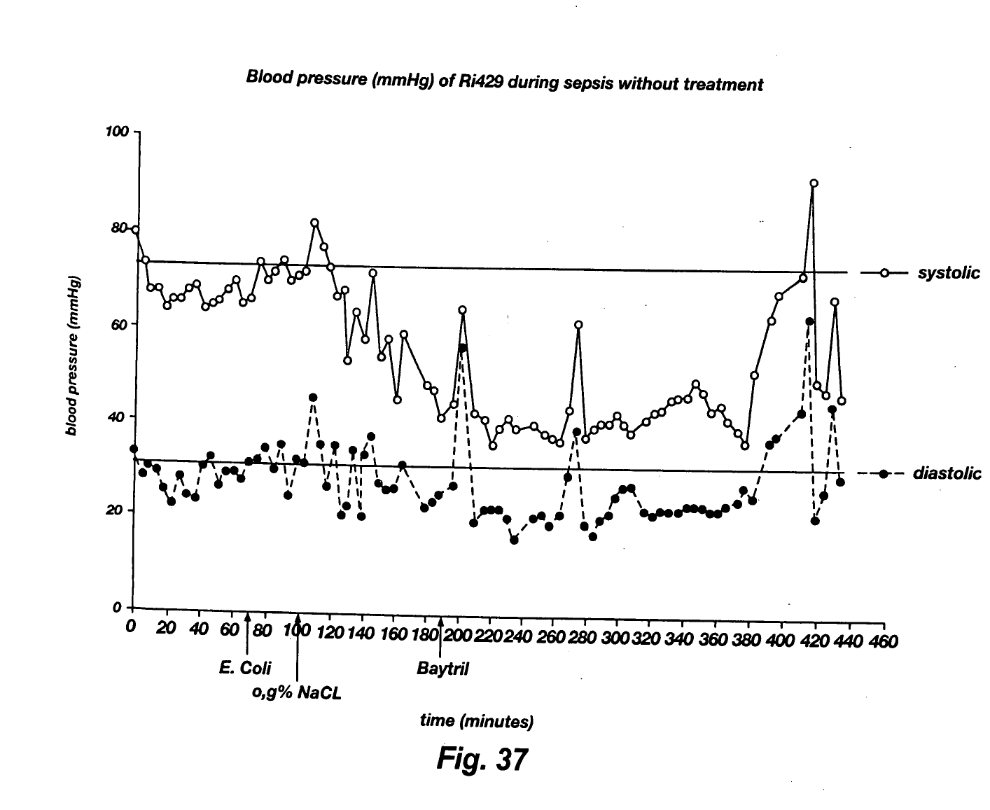

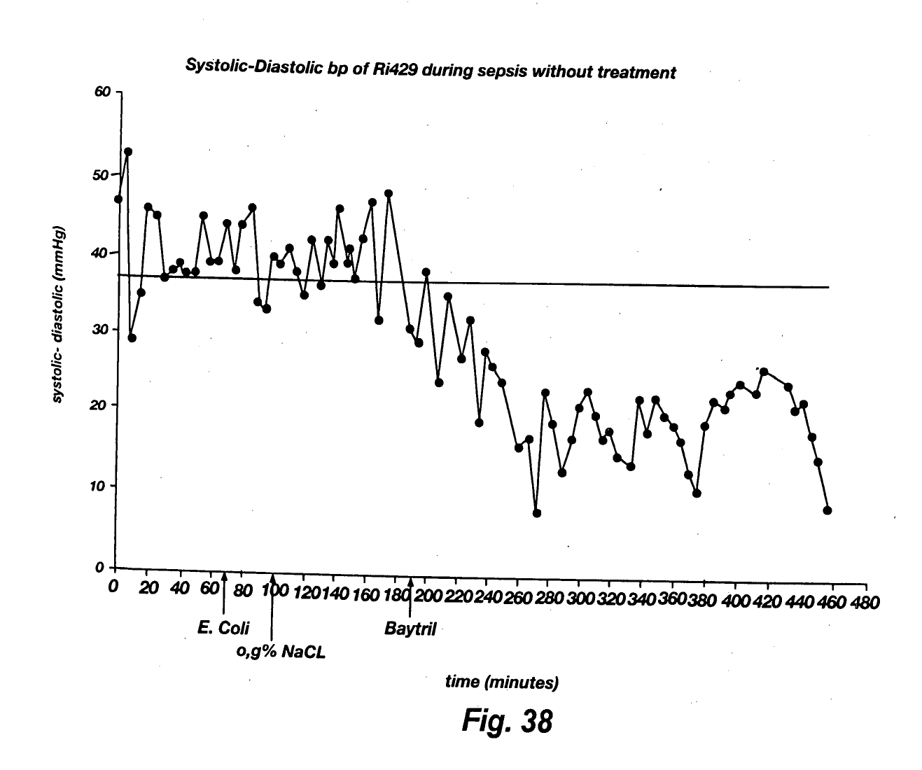

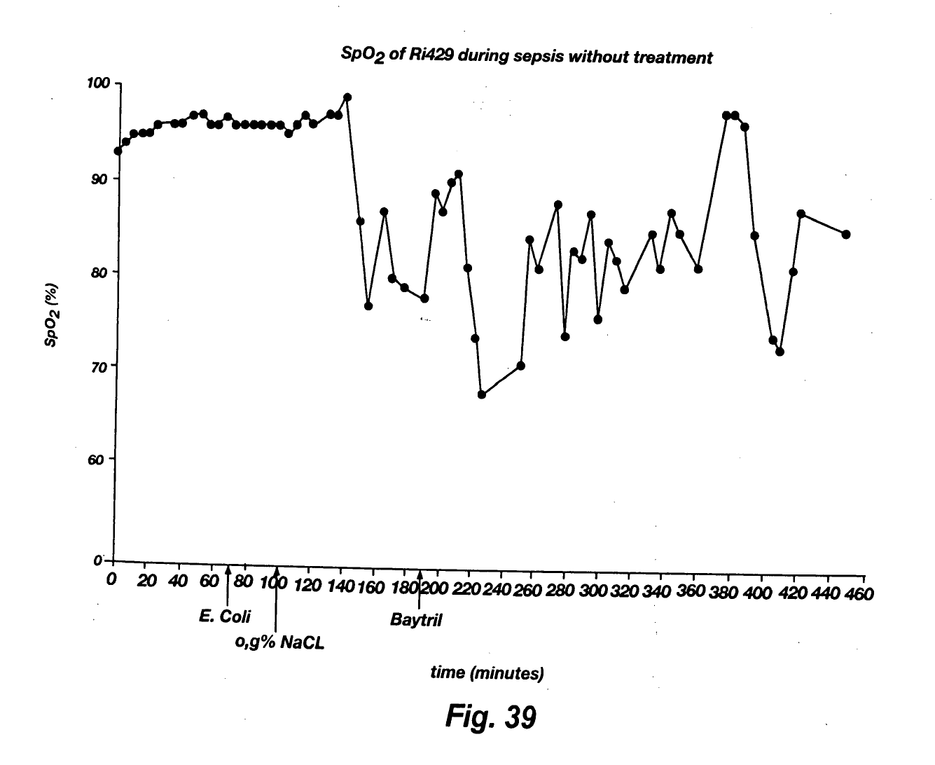

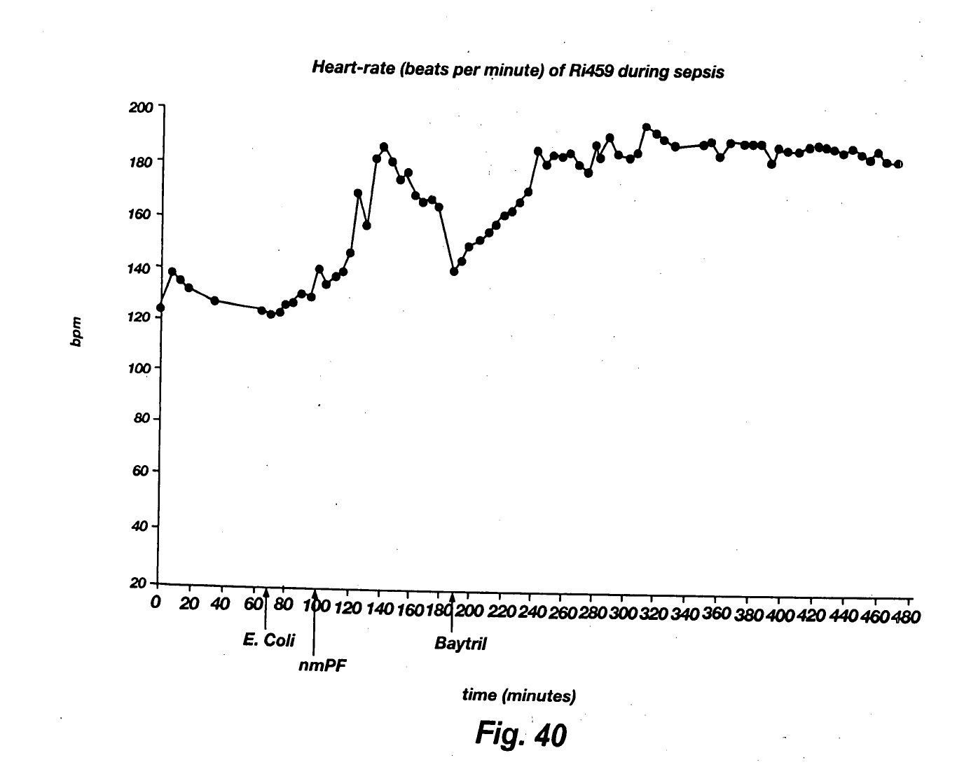

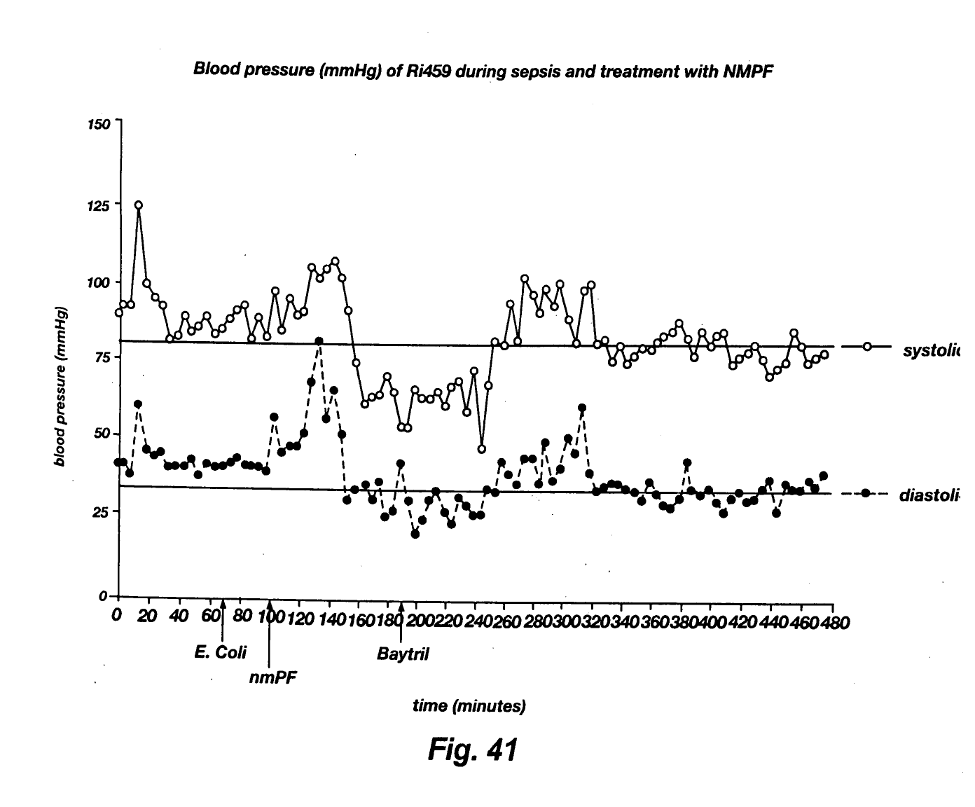

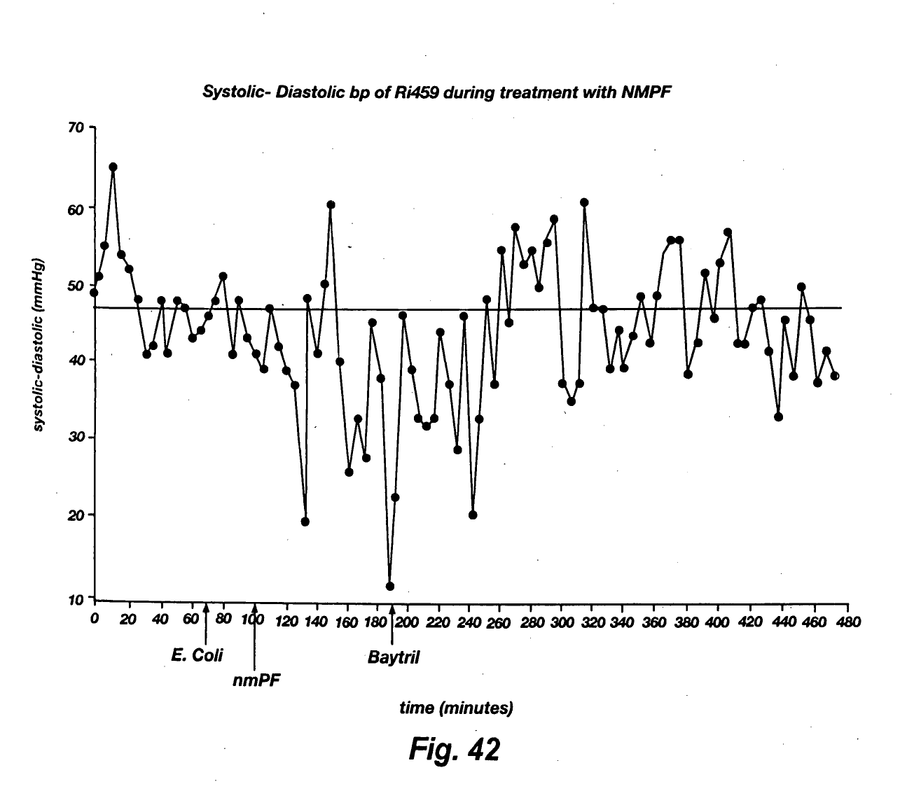

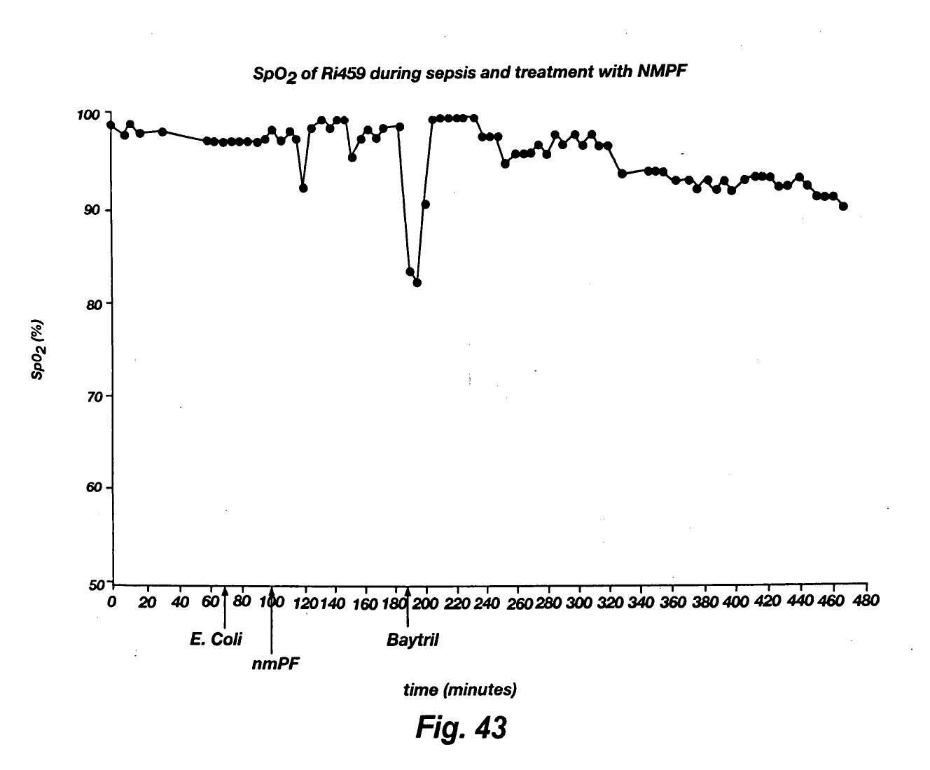

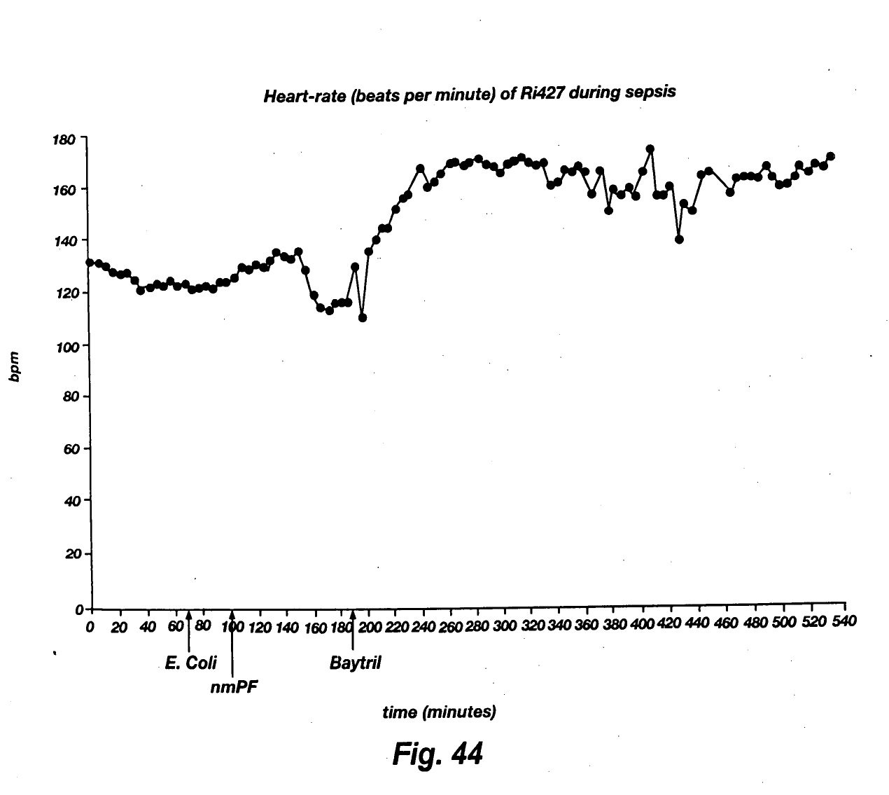

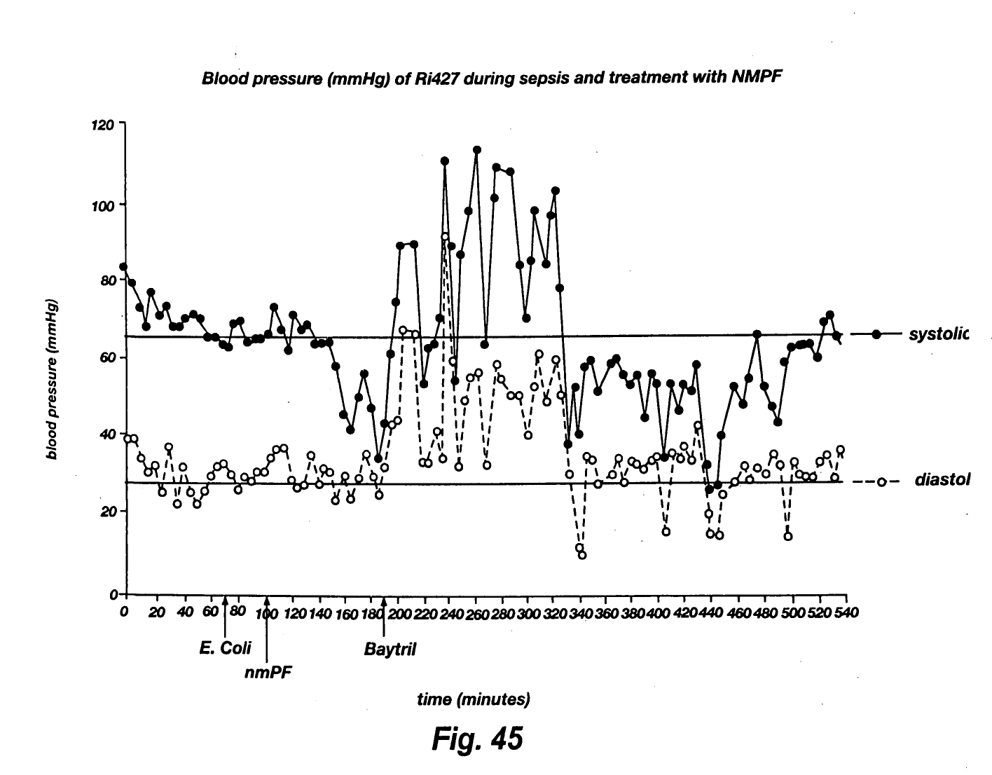

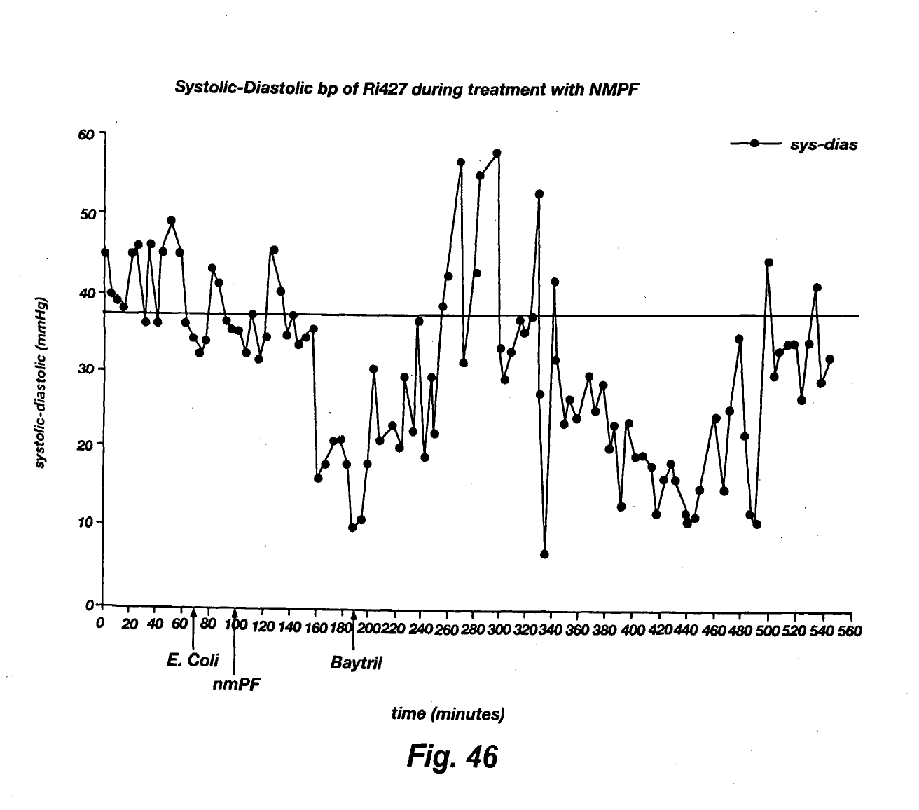

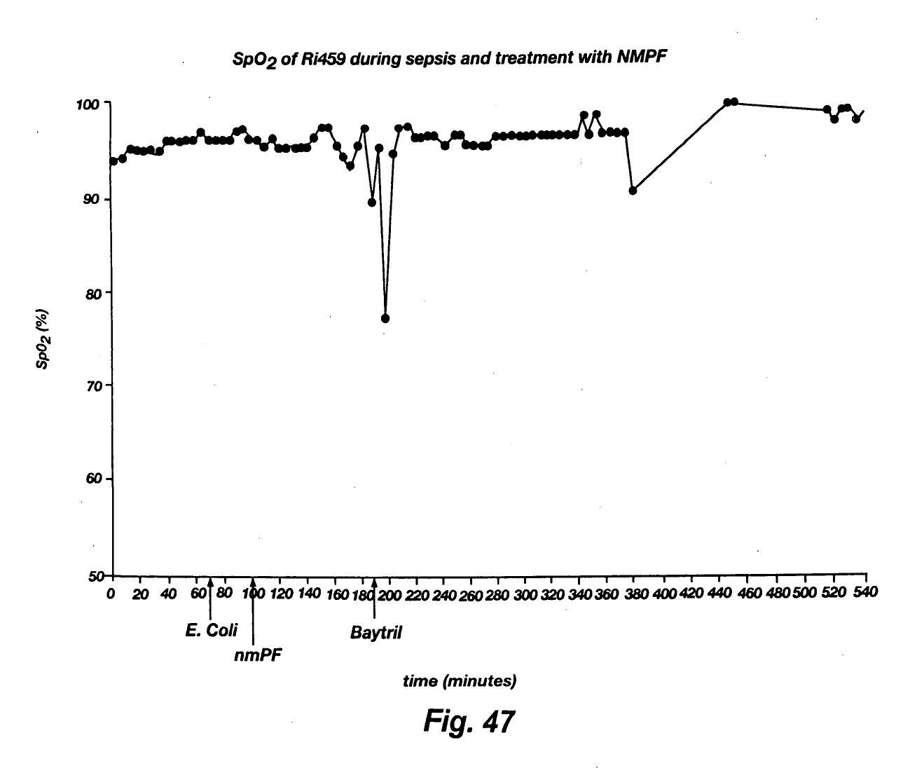

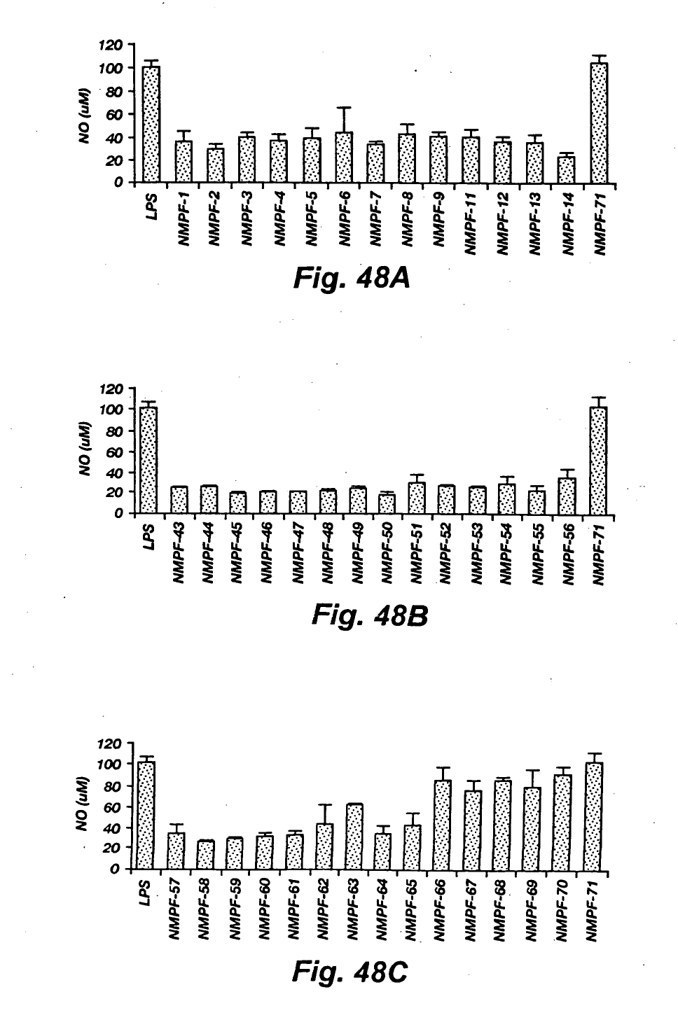

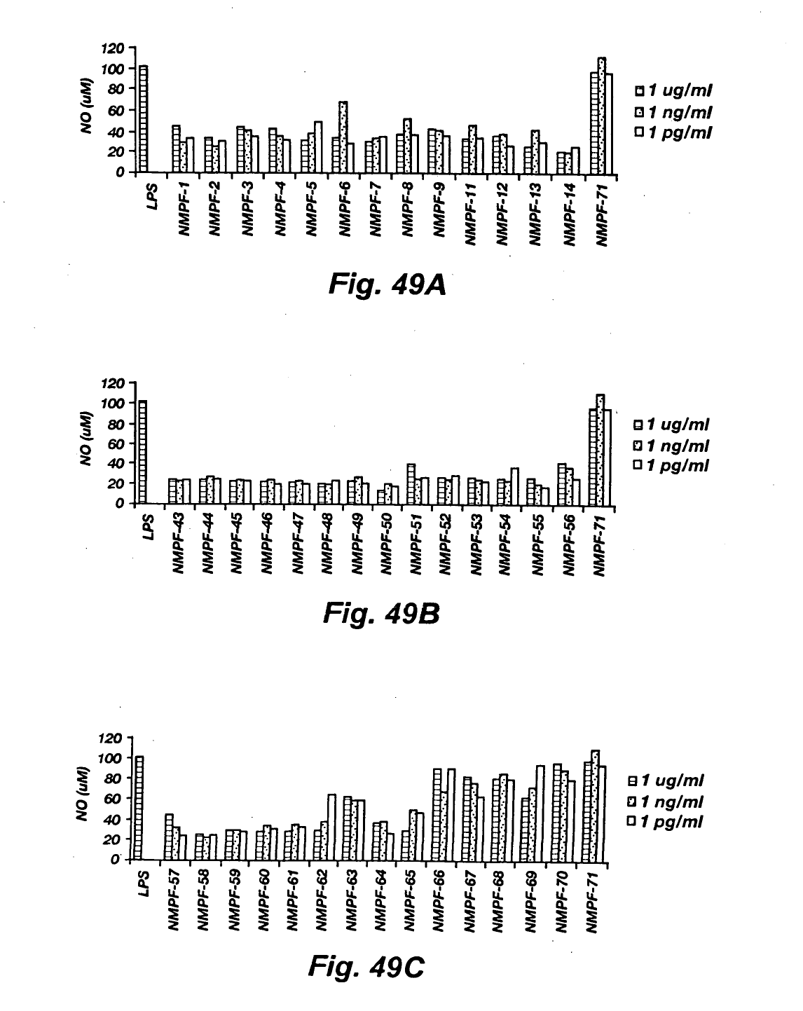

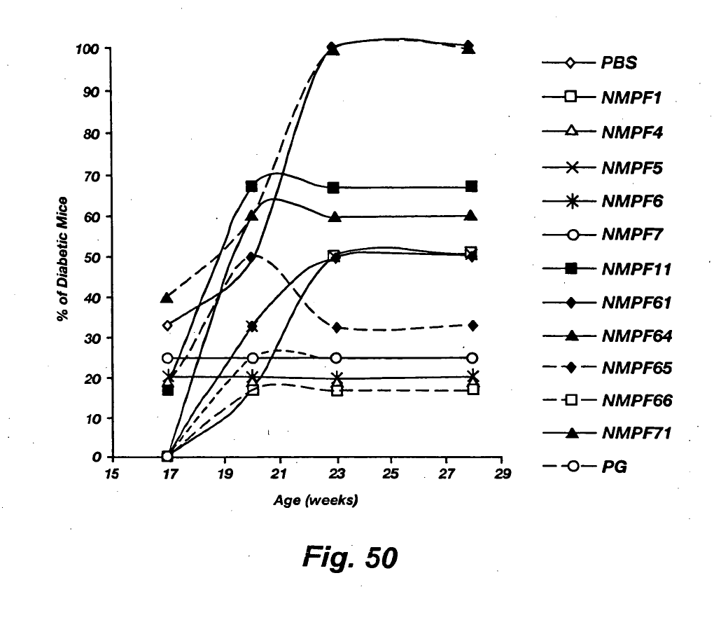

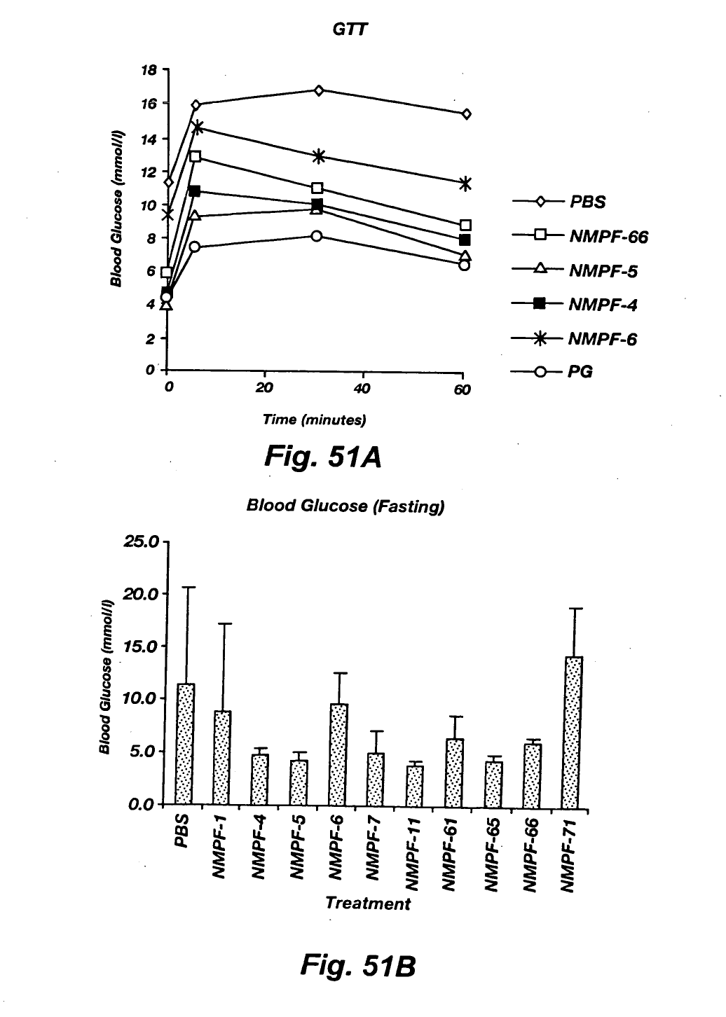

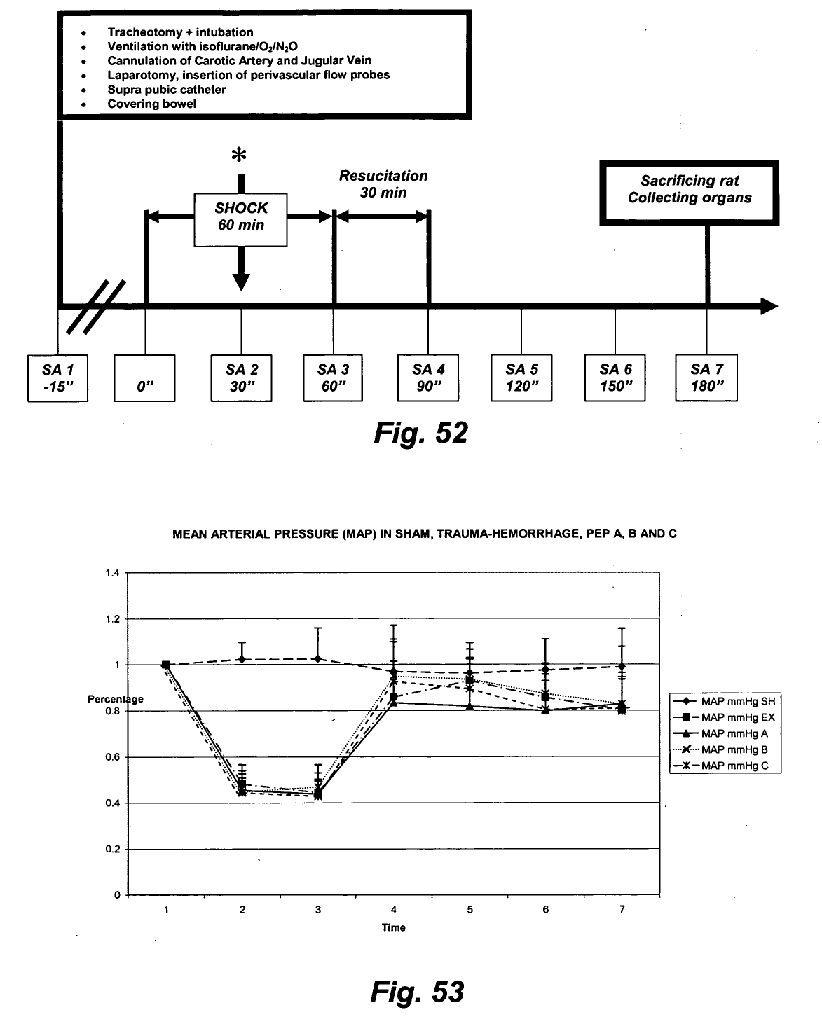

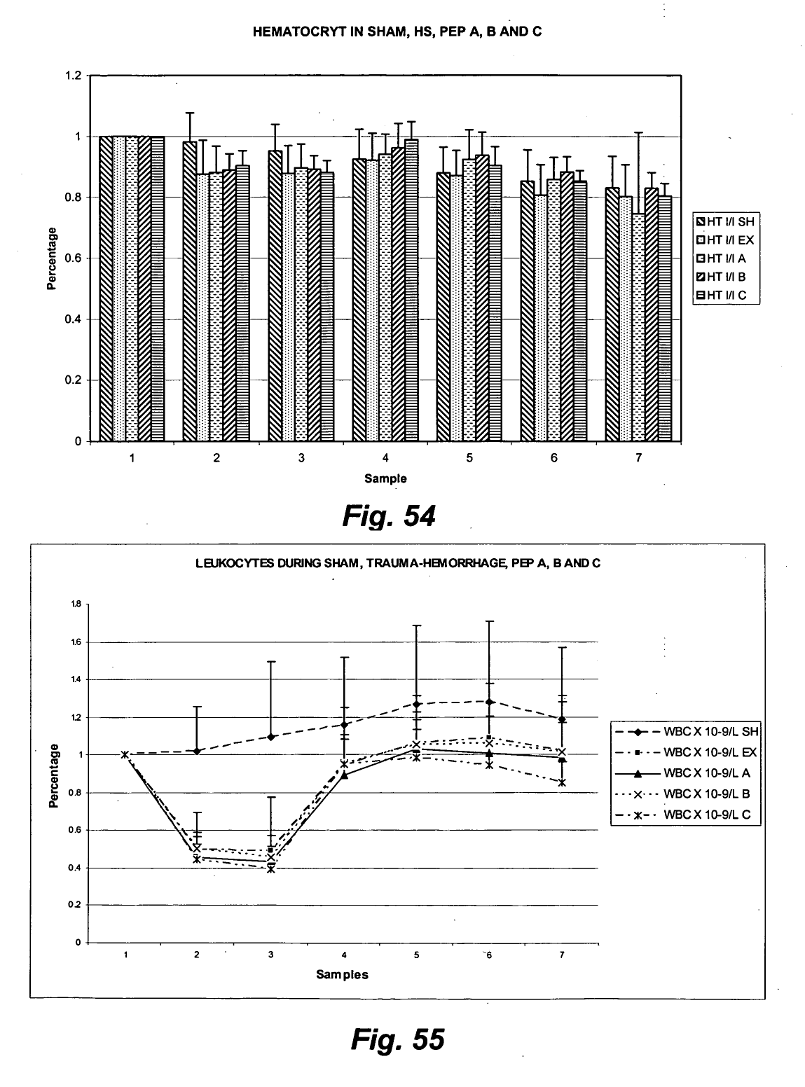

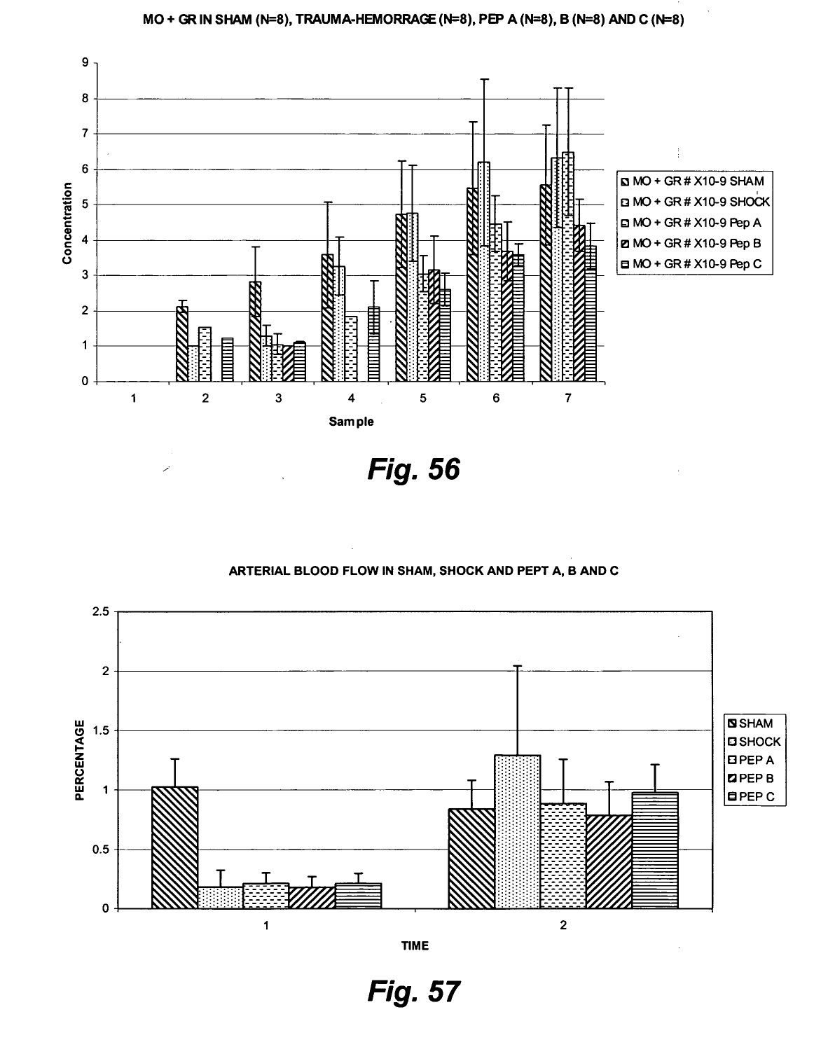

The molecular weight of this peptide is around 416 dalton. Besides 416 dalton mass, FIG. 35 shows some other molecular weights. This is to be explained by the high concentration of the peptide which induces the formation of dimers and sodium-adducts (m/z 416-[M+H]+, 438-[M+Na]+, 831-[2M+H]+, 853-[2M+Na]+, 1245-[3M+H]+, 1257-[3M+Na]+). FIG. 36 shows the MS results of 10 to 15 minutes fraction of nuclear extract obtained from LPS-stimulated cells. These results show the absence of 416 dalton mass, while FIG. 37 shows the presence of 416 dalton mass of which the MSn data (FIG. 37) and MS-sequence confirm the presence of LQGV (SEQ ID NO:1) peptide in the nuclear protein extract obtained from LQGV+LPS-stimulated RAW264.7 cells.

### Endotoxin Shock Model (Sepsis)

Sepsis. For the endotoxin model, BALB/c mice were injected i.p. with 8 to 9 mg/kg LPS (E. coli 026:B6; Difco Lab., Detroit, Mich., USA). Control groups (PBS) were treated with PBS i.p. only. To test the effect of NMPF from different sources (synthetic, commercial hCG preparation [c-hCG]), we treated BALB/c mice with a dose of 300 to 700 IU of different hCG preparations (PG23; Pregnyl batch no. 235863, PG25; Pregnyl batch no. 255957) and with synthetic peptides (5 mg/kg) after two hours of LPS injection. In other experiments, BALB/c mice were injected i.p. either with 10 mg/kg or with 11 mg/kg LPS (E. coli 026:B6; Difco Lab., Detroit, Mich., USA). Subsequently, mice were treated after two hours and 24 hours of LPS treatment with NMPF peptides.

Semi-quantitative sickness measurements. Mice were scored for sickness severity using the following measurement scheme:

1 Percolated fur, but no detectable behavior differences compared to normal mice.

2 Percolated fur, huddle reflex, responds to stimuli (such as tap on cage), just as active during handling as healthy mouse.

3 Slower response to tap on cage, passive or docile when handled, but still curious when alone in a new setting.

4 Lack of curiosity, little or no response to stimuli, quite immobile.

5 Labored breathing, inability or slow to self-right after being rolled onto back (moribund).

6 Sacrificed.

### Results

### Endotoxin Shock Model (Sepsis)

Sepsis experiments. To determine the effect of synthetic peptides (NMPF) in high-dose LPS shock model, BALB/c mice were injected intraperitoneally with different doses of LPS and survival was assessed daily for five days. In this experiment (for the LPS endotoxin model), BALB/c mice were injected i.p. with 8 to 9 mg/kg LPS (E. coli 026:B6; Difco Lab., Detroit, Mich., USA). Control groups (PBS) were treated with PBS i.p. only. We treated BALB/c mice with a dose of 300 to 700 IU of different hCG preparations (PG23; PREGNYL® batch no. 235863, PG25; PREGNYL® batch no. 255957) or with peptides (5 mg/kg) after two hours of LPS injection.

These experiments showed (Table 1) that NMPF peptides 4, 6, 66 and PG23 inhibited shock completely (all mice had in first 24 hours sickness scores not higher than two; shortly thereafter they recovered completely and had sickness scores of zero), while peptides 2, 3 and 7 accelerated shock (all mice had in first 24 hours sickness scores of five and most of them died, while the control mice treated with LPS+PBS had sickness scores of three to four in first 24 hours and most of them died after 48 hours with sickness scores of five (17% survival rate at 72 hours). In addition, peptides 1, 5, 8, 9, 11, 12, 13, 14 and 64 showed in a number of different experiments variability in effectiveness as well as in the kind (inhibitory vs. accelerating) of activity. This variability is likely attributable to the rate of breakdown of the various peptides and the different effects the various peptides and their breakdown products have in vivo. In addition, these experiments also showed the variability in anti-shock activity in c-hCG preparations that is likely attributable to the variation in the presence of anti-shock and shock-accelerating NMPF. Visible signs of sickness were apparent in all of the experimental animals, but the kinetics and obviously the severity of this sickness were significantly different. These data are representative of at least ten separate experiments.

In Table 2, we see the effect of ALA-replacement (PEPSCAN) in peptides LQG, LQGV (SEQ ID NO:1), VLPALP (SEQ ID NO:3), and VLPALPQ (SEQ ID NO:29) in septic shock experiments. We conclude that the change in even one amino acid by a neutral amino acid can lead to different activity. So, genomic differences as well as polymorphism in these peptides can regulate the immune response very precisely. Derivatives of these peptides, for example (but not limited to), by the addition of classical and non-classical amino acids or derivatives that are differentially modified during or after synthesis; for example, benzylation, amidation, glycosylation, proteolytic cleavage, linkage to an antibody molecule or other cellular ligand, etc., could also lead to a better effectiveness of the activity.

To determine whether treatment of BALB/c mice with NMPF inhibits septic shock at different stages of disease, synthetic peptides (NMPF) were injected i.p. at two and 24 hours after the induction of septic shock with high dose LPS (10 mg/kg).

As shown in Tables 3 and 4, control mice treated with PBS after the shock induction reached a sickness score of five at 14 and 24 hours, and remained so after the second injection with PBS. The survival rate in control group mice was 0% at 48 hours. In contrast to control mice, mice treated with NMPF-9, 11, 12, 43, 46, 50 and 60 reached a maximum sickness score of two to three at 24 hours after the induction of septic shock and further reached a maximum sickness score of one to two at 48 hours after the second injection of NMPF. In addition, mice treated with NMPF-5, 7, 8, 45, 53 and 58 reached a sickness score of five and after the second injection with NMPF all mice returned to a sickness score of one to two and survival rates in NMPF groups were 100%. Mice treated with NMPF-3 reached sickness scores of three to four and the second NMPF injection did save these mice. These experiments show that NMPF peptides have anti-shock activity at different stages of the disease and NMPF have anti-shock activity even at a disease stage when otherwise irreversible damage had been done. This indicates that NMPF have effects on different cellular levels and also have repairing and regenerating capacity.

### Dendritic Cells Experiments

Mice. The mouse strain used in this study was BALB/c (Harlan, Bicester, Oxon, GB). All mice used in experiments were females between eight and twelve weeks of age. Mice were housed in a specific pathogen-free facility. The Animal Use Committee at the Erasmus University Rotterdam, NL approved all studies.

In vivo treatment. At least six mice per group were injected intraperitoneally (i.p) with LPS (10 mg/kg; Sigma). After two and 24 hours of LPS induction, mice were injected i.p. with either NMPF (5 mg/kg) or PBS, in a volume of 100 μl. LPS-induced shock in this model had more than 90% mortality at 48 hours.

Bone marrow cell culture. From treated mice, bone marrow cells were isolated and cultured as follows. BALB/c mice were killed by suffocation with CO₂. The femurs and tibiae were removed and freed of muscles and tendons under aseptic conditions. The bones were placed in R10 medium (RPMI 1640, supplemented with 50 U/ml penicillin, 50 μg/ml streptomycin, 0.2 M Na-pyruvate, 2 mM glutamine, 50 μM 2-mercaptoethanol and 10% fetal calf serum (Bio Whittaker, Europe)).

The bones were then cleaned more thoroughly by using an aseptic tissue and were transferred to an ice cold mortier with 2 ml of R10 medium. The bones were crushed with a mortel to get the cells out of the bones. Cells were filtered through a sterile 100 μM filter (Beckton Dickinson Labware) and collected in a 50 ml tube (FALCON). This procedure was repeated until bone parts appeared translucent.

The isolated cells were resuspended in 10 ml of R10 and 30 ml of Geys medium was added. The cell suspension was kept on ice for 30 minutes to lyse the red blood cells. Thereafter, the cells were washed twice in R10 medium. Upon initiation of the culture, the cell concentration was adjusted to 2×10⁵ cells per ml in R10 medium supplemented with 20 ng/ml recombinant mouse Granulocyte Monocyte-Colony Stimulating Factor (rmGM-CSF; BioSource International, Inc., USA) and seeded in 100 mm non-adherent bacteriological Petri dishes (Falcon). For each condition, six Petri dishes were used and for further analysis, cells were pooled and analyzed as described ahead. The cultures were placed in a 5% CO₂-incubator at 37° C. Every three days after culture initiation, 10 ml fresh R10 medium supplemented with rmGM-CSF at 20 ng/ml was added to each dish.

Nine days after culture initiation, non-adherent cells were collected and counted with a Coulter Counter (Coulter).

Alternatively, BM cells from untreated mice were isolated and cultured as described above and were in vitro treated with the following conditions: NMPF-4, NMPF-46, NMPF-7, NMPF-60 (20 μg/ml) were added to the culture either at day 0 or day 6 after culture initiation, or LPS (1 μg/ml) was added to the culture at day 6 with or without the NMPF.

Immunofluorescence staining. Cells (2×10⁵) were washed with FACS-buffer (PBS with 1% BSA and 0.02% sodium azide) and transferred to a round-bottomed 96-well plate (NUNC). The antibodies used for staining were against MHC-II (I-A/I-E) PE and CD11c/CD18 FITC (PharMingen/Becton Dickinson, Franklin Lakes, N.J., USA).

Cells were resuspended in 200 μl FACS-buffer containing both of the antibodies at a concentration of 2.5 ng/μl per antibody. Cells were then incubated for 30 minutes at 4° C. Thereafter, cells were washed three times and finally resuspended in 200 μl FACS-buffer for flow-cytometric analysis in a FACSCalibur flow cytometer (Becton Dickinson, Heidelberg, DE). All FACS-data were analyzed with CellQuest software (Becton Dickinson, Heidelberg, DE).

Statistical analysis. All differences greater than 20% are considered to be significant.

### Results (Dendritic Cell Experiments)

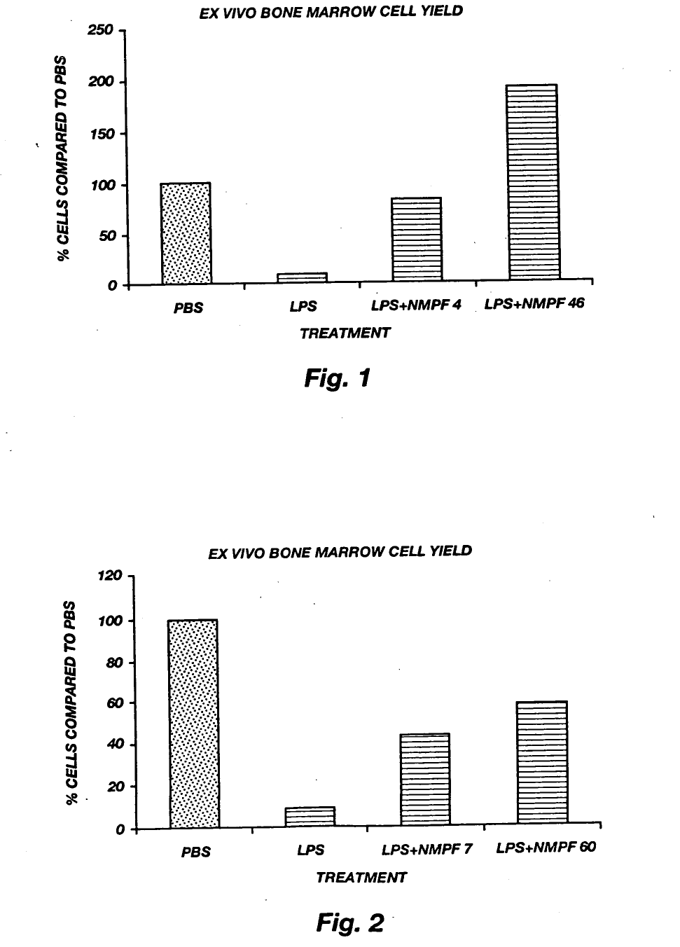

Cell yield of ex vivo bone marrow cell cultures. To determine the ex vivo effect of LPS and NMPF treatment on the cell yield obtained from a nine-day culture of bone marrow with rmGM-CSF, cells were isolated from the BM of treated mice and cultured, harvested and counted as described. As shown in FIGS. 1 and 2, the cell yield of the bone marrow cultures of LPS- (10 mg/kg) treated mice is significantly decreased compared to PBS-treated mice. Mice treated with NMPF-4, NMPF-7, NMPF-46 and NMPF-60 after LPS shock induction had a significantly increased cell yield compared to LPS in the presence of rmGM-CSF. In addition, BM cultures from NMPF-46-treated mice gave a significantly increased cell yield even compared to the PBS group.

Immunofluorescence staining of in vivo-treated bone marrow-derived DC. Culture of BM cells in the presence of rmGM-CSF gave rise to an increased population of cells that are positive for CD11c and MHC-II. Cells positive for these cell membrane markers are bone marrow-derived dendritic cells (DC). DC are potent antigen-presenting cells (APC) and modulate immune responses. In order to determine the maturation state of myeloid-derived DC, cells were stained with CD11c and MHC-II.

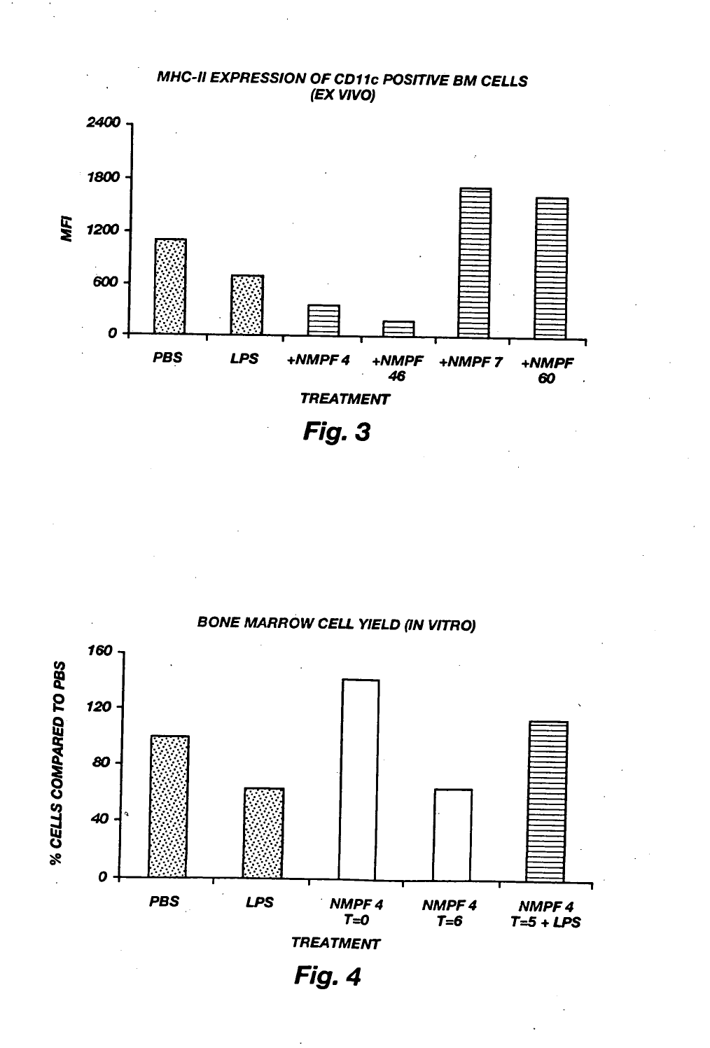

As shown in FIG. 3, the expression of the MHC-II molecule was significantly decreased on CD11c-positive cells from LPS-treated mice as compared to the PBS group. This decrease in MHC-II expression was further potentiated by the in vivo treatment with NMPF-4 and NMPF-46. However, treatment of mice with NMPF-7 and NMPF-60 significantly increased the expression of the MHC-II molecule even as compared to the PBS group.

Cell yields of in vitro bone marrow cell cultures. To determine the effect of LPS and NMPF in vitro on the cell yield of a nine-day culture of bone marrow cells, we isolated the BM cells from untreated BALB/c mice and cultured them in the presence of rmGM-CSF. In addition to rmGM-CSF, cultures were supplemented with NMPF at either day 0 or day 6 with or without the addition of LPS at day 6.

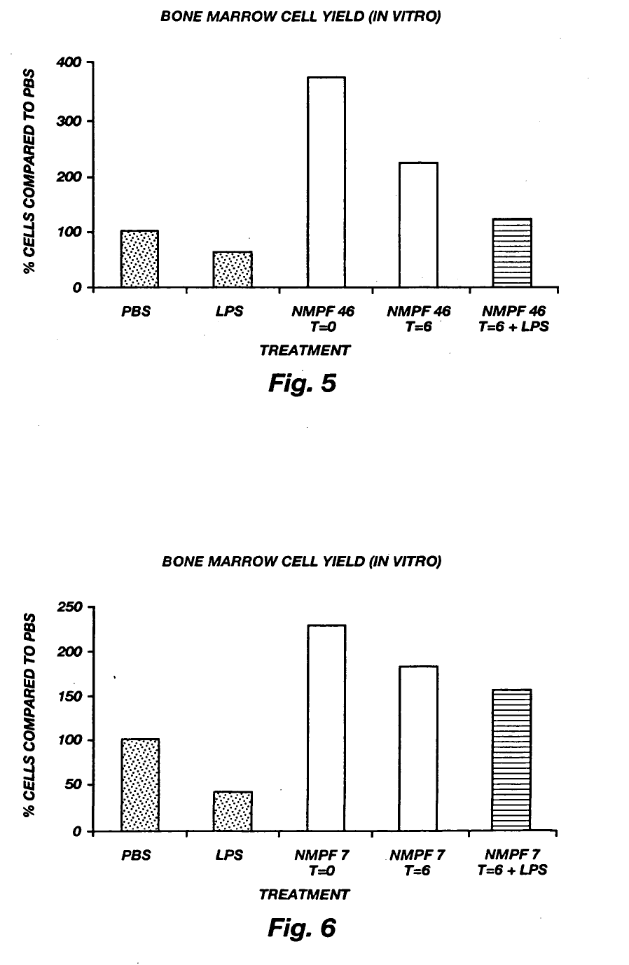

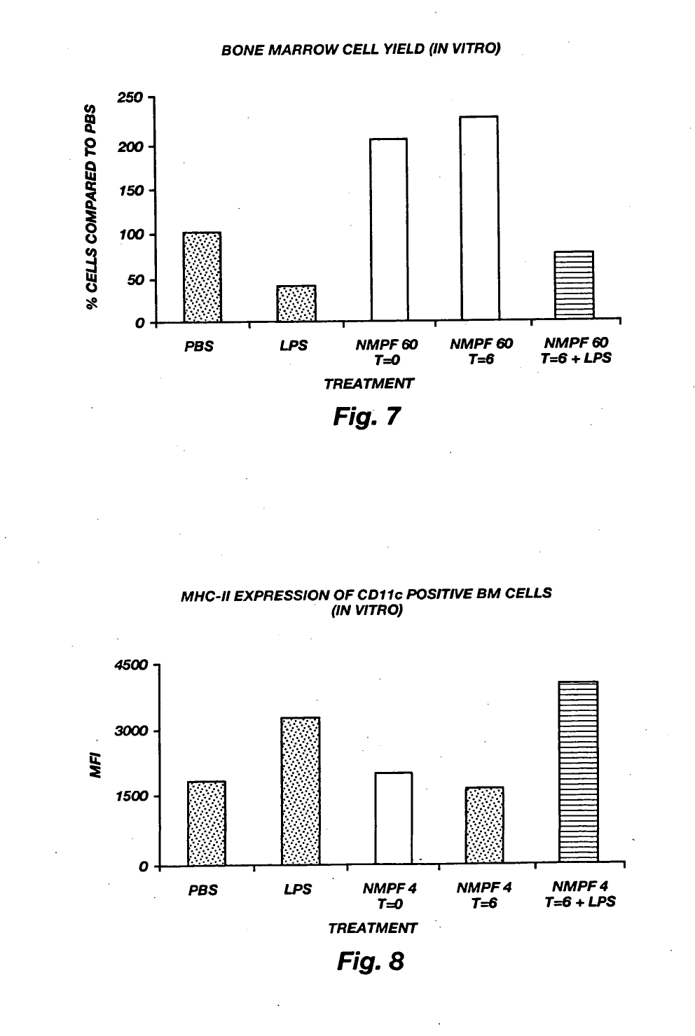

As shown in FIGS. 4-7, there is a significant decrease in cell yield in LPS-treated BM cells as compared to PBS. BM cells treated with NMPF-4, 7, 46 or 60 at time point t=0 or t=6 without LPS showed a significant increase in cell yield as compared to the PBS group. However, BM cell cultures treated with NMPF-4 at time point t=6 showed significant decrease in cell yield as compared to the PBS group and this effect is comparable with the effect of LPS (FIG. 4). In addition, BM cells treated with NMPF-4, 7, 46 or 60 at time point t=6 in combination with LPS showed a significant increase in cell yield as compared to the LPS group, and even in the group of NMPF-7, the cell yield was significantly increased as compared to the PBS group.

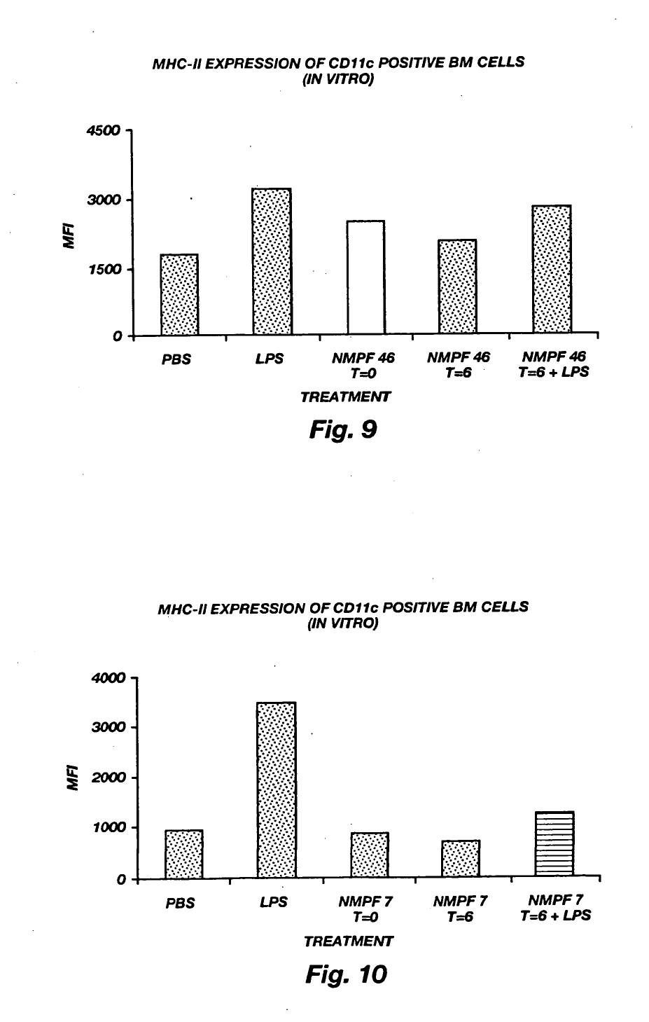

Immunofluorescence staining of in vitro-treated bone marrow-derived DC. To determine the maturation state of DC, CD11c positive cells were stained for MHC-II antibody. FIGS. 8 through 11 show that there is an opposite effect of LPS on MHC-II expression as compared to in vivo experiments, namely, MHC-II expression is significantly increased with LPS treatment in vitro as compared to PBS. NMPF-4 with LPS further potentiated the effect of LPS, while NMPF-7 with or without LPS (t=6) significantly inhibited the expression of MHC-II as compared to LPS and PBS, respectively. However, cells treated with NMPF-46 without LPS (t=0) showed significantly increased expression of MHC-II on CD11c positive cells. Furthermore, no significant differences were found in the group treated with NMPF-60 with or without LPS on MHC-II expression as compared to LPS- and PBS-treated cells.

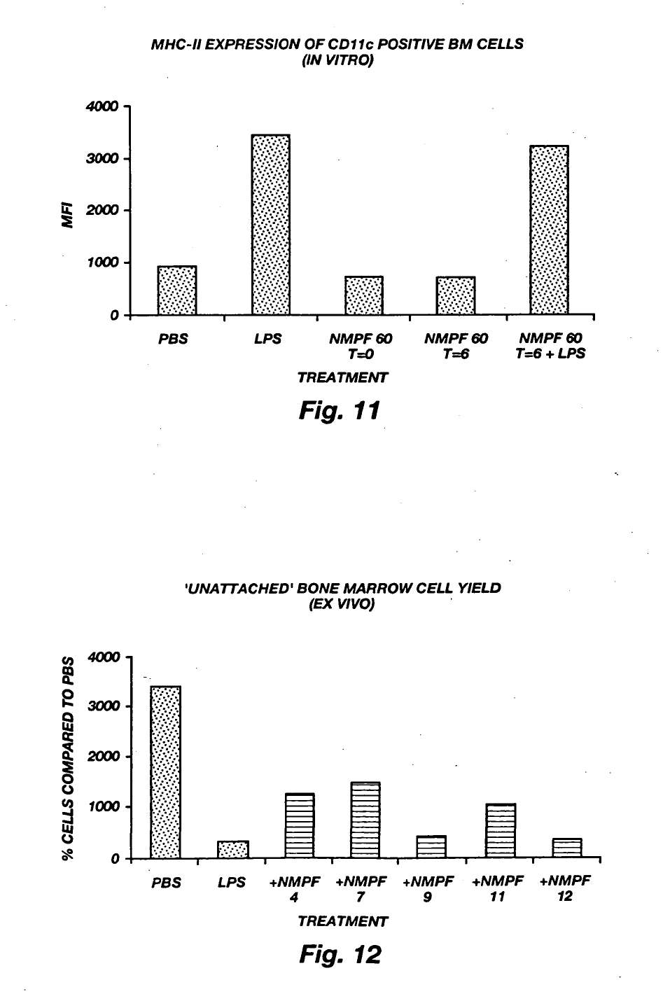

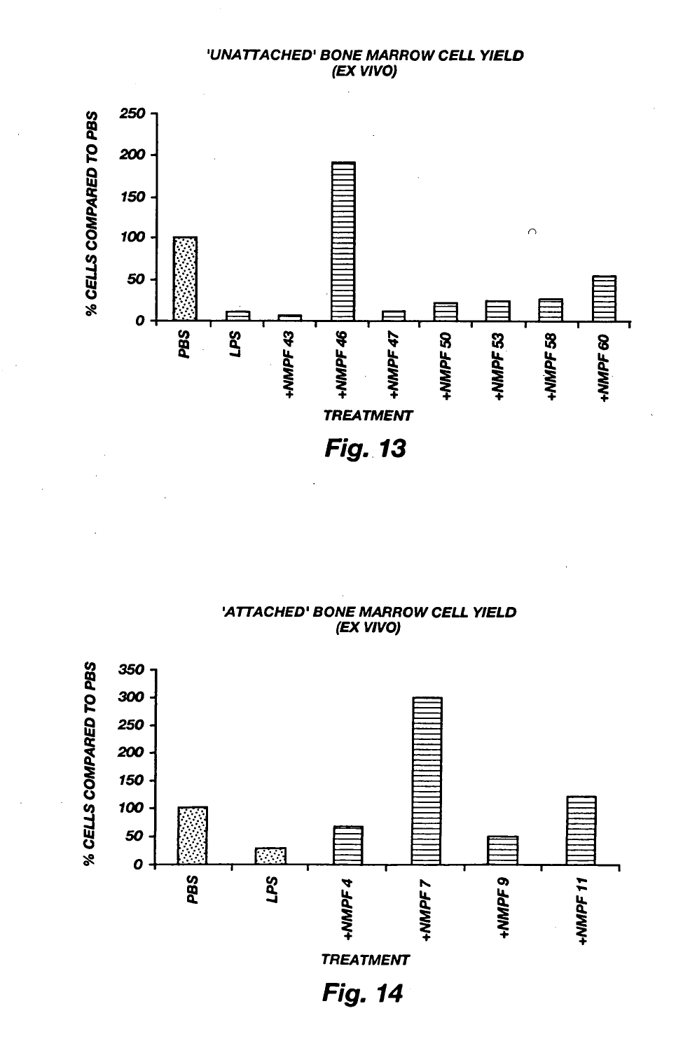

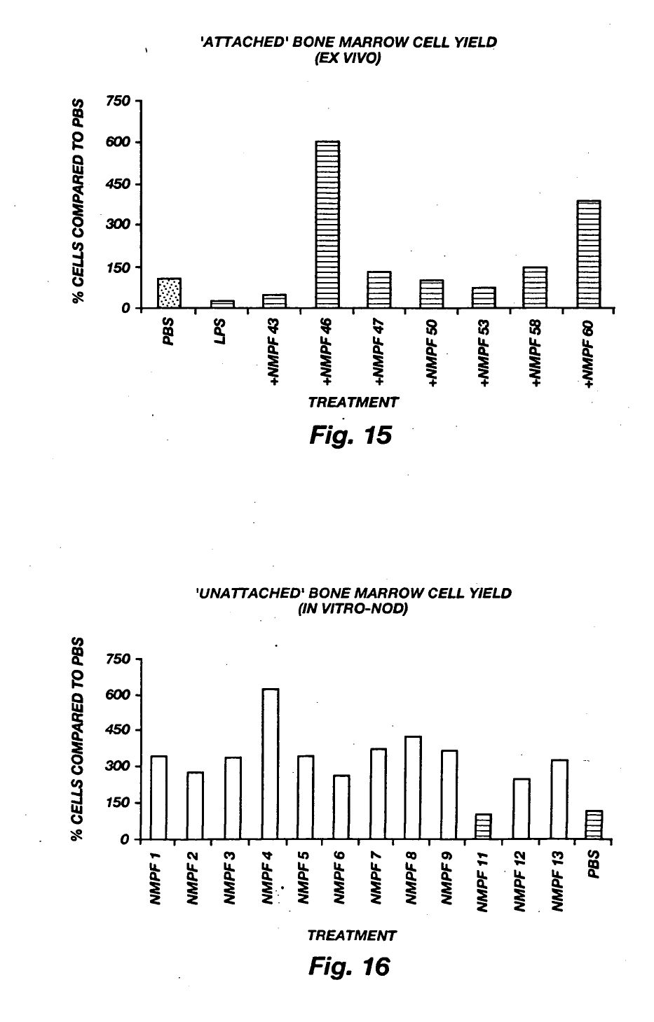

To determine the in vivo effect of LPS and NMPF treatment on the cell yield obtained from a nine-day culture of bone marrow with rmGM-CSF, cells were isolated from the BM of treated mice and cultured, harvested and counted as described. The cell yield of “attached” cells was significantly increased with NMPF-4, 7, 9, 11, 43, 46, 47, 50, 53, 58 60, and even in the group of NMPF-7, 46 and 60, the cell yield was significantly increased as compared to the PBS group (FIGS. 14 and 15). In addition, cell yield of “unattached” cells was significantly increased with NMPF-4, 7, 9, 11, 46, 50, 53, 58 60, and again in the group of NMPF-46, the cell yield was significantly increased as compared to the PBS group (FIGS. 12 and 13).

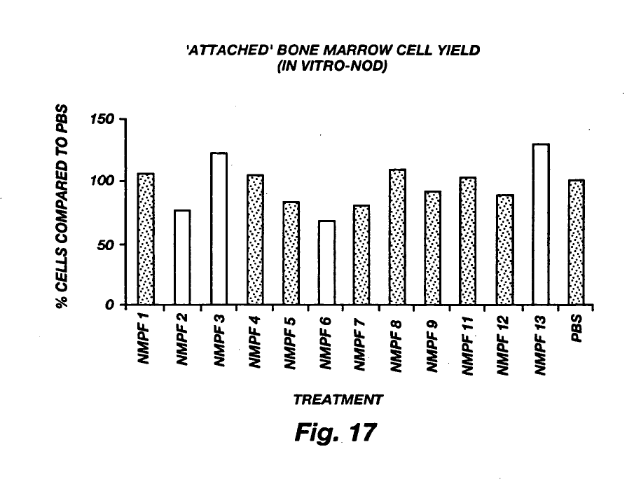

To determine the effect of LPS and NMPF in vitro on the cell yield of a nine-day culture of bone marrow cells of female NOD mice, we isolated the BM cells from untreated NOD mice and cultured them in the presence of rmGM-CSF. In addition to rmGM-CSF, cultures were supplemented with NMPF. In these experiments, the bone marrow cell yield of “unattached” cells was significantly increased with NMPF-1, 2, 3, 4, 5, 6, 7, 8, 9, 12 and 13 as compared to the PBS group, and no effect was observed with NMPF-11 (FIG. 16). The “attached” bone marrow cells of these experiments showed different yield than the “un-attached” cells, namely, there was a significant increase in cell yield in cultures treated with NMPF-3 and 13, while cultures treated with NMPF-2 and 6 showed significant decrease in the cell yield as compared to PBS (FIG. 17) (additional results are summarized in Table 5).

### Coronary Artery Occlusion (CAO) Experiments

CAO induction and treatment. NMPF have immunoregulatory effects in chronic inflammatory and acute inflammatory mice models. Since certain cytokines like TGF-β1, TNF-α, IL-1 and ROS (reactive oxygen species) have been implicated in irreversible myocardial damage produced by prolonged episodes of coronary artery occlusion and reperfusion in vivo leading to ischemia and myocardial infarct, we tested the cardio-protective properties of peptides in ad libitum fed male Wistar rats (300 g). The experiments were performed in accordance with the Guiding Principles in the Care and Use of Animals as approved by the Council of the American Physiological Society and under the regulations of the Animal Care Committee of the Erasmus University Rotterdam. Rats (n=3) were stabilized for 30 minutes followed by i.v. with 1 ml of peptide treatment (0.5 mg/ml) in ten minutes. Five minutes after completion of treatment, rats were subjected to a 60-minute coronary artery occlusion (CAO). In the last 5 minutes of CAO, rats were again treated over ten minutes by i.v. with 1 ml of peptide (0.5 mg/ml) followed by 2 hours of reperfusion (IP). Experimental and surgical procedures are described in detail in Cardiovascular Research 37 (1998) 76-81. At the end of each experiment, the coronary artery was re-occluded and was perfused with 10 ml Trypan Blue (0.4%, Sigma Chemical Co.) to stain the normally perfused myocardium dark blue and delineate the nonstained area at risk (AR). The heart was then quickly excised and cut into slices of 1 mm from apex to base. From each slice, the right ventricle was removed and the left ventricle was divided into the AR and the remaining left ventricle, using micro-surgical scissors. The AR was then incubated for ten minutes in 37° C. Nitro-Blue-Tetrazolium (Sigma Chemical Co.; 1 mg per 1 ml Sorensen buffer, pH 7.4), which stains vital tissue purple but leaves infarcted tissue unstained. After the infarcted area (IA) was isolated from the noninfarcted area, the different areas of the LV were dried and weighed separately. Infarct size was expressed as percentage of the AR. Control rats were treated with PBS.

### Results (Coronary Artery Occlusion (CAO) Experiments)

Our CAO data showed that 15 rats in the control group treated with only PBS had an infarcted area of 70±2% (average±standard error) after 60 minutes of CAO followed by two hours of reperfusion, while rats treated with peptides VLPALP (SEQ ID NO:3), LQGV (SEQ ID NO:1), VLPALPQVVC (SEQ ID NO:20), LQGVLPALPQ (SEQ ID NO:49), LAGV (SEQ ID NO:26), LQAV (SEQ ID NO:52) and MTRV (SEQ ID NO:42), showed an infarcted area of 62±6%, 55±6%, 55±5%, 67±2%, 51±4%, 62±6% and 68±2%, respectively. Here, we see that certain peptides (such as VLPALP (SEQ ID NO:3), LQGV (SEQ ID NO:1), VLPALPQVVC (SEQ ID NO:20), LAGV (SEQ ID NO:26)) have a protective effect on the area at risk for infarction. In addition, peptide LQAV (SEQ ID NO:52) showed a smaller infarcted area but, in some instances, the area was hemorrhagic infarcted. In addition, NMPF-64 (LPGCPRGVNPVVS (SEQ ID NO:40)) had also a protective effect (35%) in CAO experiments. It is important to note that mice treated with certain above-mentioned peptides showed less viscosity of blood. Apart from immunological effect, these peptides may have also an effect on the blood coagulation system directly or indirectly since there is certain homology with blood coagulation factors (for additional results of NMPF peptides see Table 5). So, in both models, the circulatory system plays an important role in the pathogenesis of the disease.

### Chicken Egg Experiments

In vivo treatment of fertilized chicken eggs with NMPF. Fertile chicken eggs (Drost Loosdrecht BV, NL) were incubated in a diagonal position in an incubator (Pas Reform BV, NL) at 37° C. and 32% relative humidity.

Solutions of NMPF peptides (1 mg/ml) and VEGF were made in PBS. At least ten eggs were injected for every condition. The treatment was performed as follows: on day 0 of incubation, a hole was drilled into the eggshell to open the air cell. A second hole was drilled 10 mm lower and right from the first hole for injection. The holes in the eggshell were disinfected with jodium. The NMPF peptides (100 μg/egg) and/or VEGF (100 ng/ml) were injected in a volume of 100 μl. The holes in the eggshell were sealed with tape (Scotch Magic™ Tape, 3M) and the eggs were placed into the incubator.

Quantification of angiogenesis. On day 7 of incubation, the eggs were viewed under a UV lamp to check if the embryos were developing in a normal way and the dead embryos were counted. On day 8 of incubation, the embryos were removed from the eggs by opening the shell at the bottom of the eggs. The shell membrane was carefully dissected and removed. The embryos were placed in a 100-mm Petri dish. The embryo and the blood vessels were photographed (Nikon E990, Japan) in vivo with the use of a microscope (Zeiss Stemi SV6, DE). One overview picture was taken and four detail pictures of the blood vessels were taken. Only eggs with vital embryos were evaluated.

Data analysis. Quantification of angiogenesis was accomplished by counting the number of blood vessel branches. Quantification of vasculogenesis was accomplished by measuring the blood vessel thickness. The number of blood vessel branches and the blood vessel thickness were counted in the pictures (four pictures/egg) using Corel Draw 7. Thereafter, the number of blood vessel branches and the thickness of the blood vessels were correlated to a raster of microscope (10 mm²) for comparison.

The mean number of branches and the mean blood vessel thickness of each condition (n=10) were calculated and compared to the PBS control eggs using a Student's T-test.

### Results (Chicken Egg Experiments)

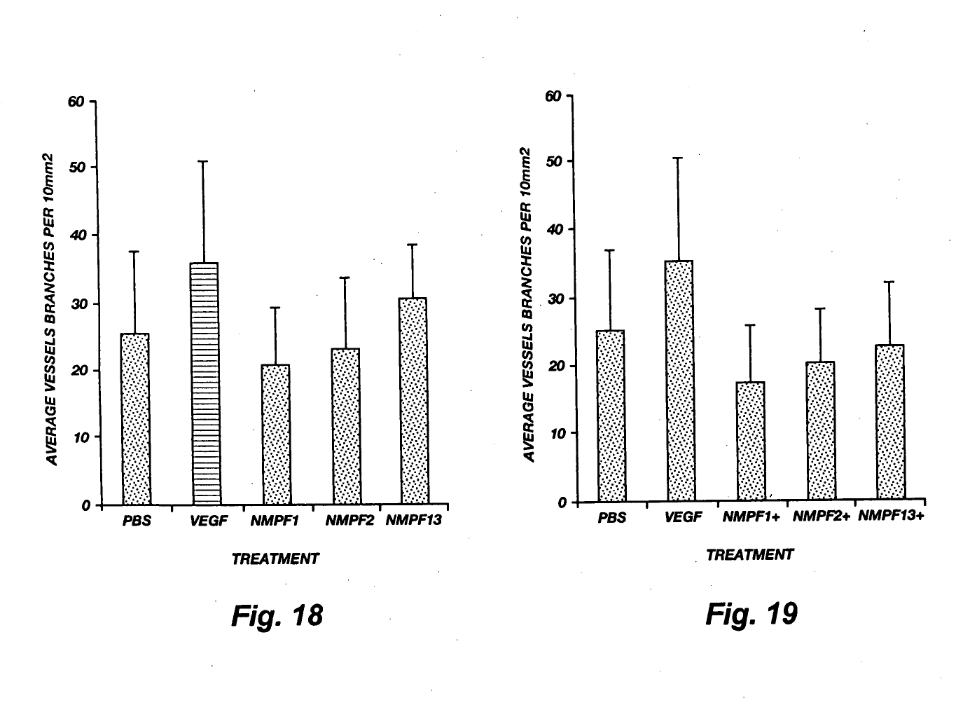

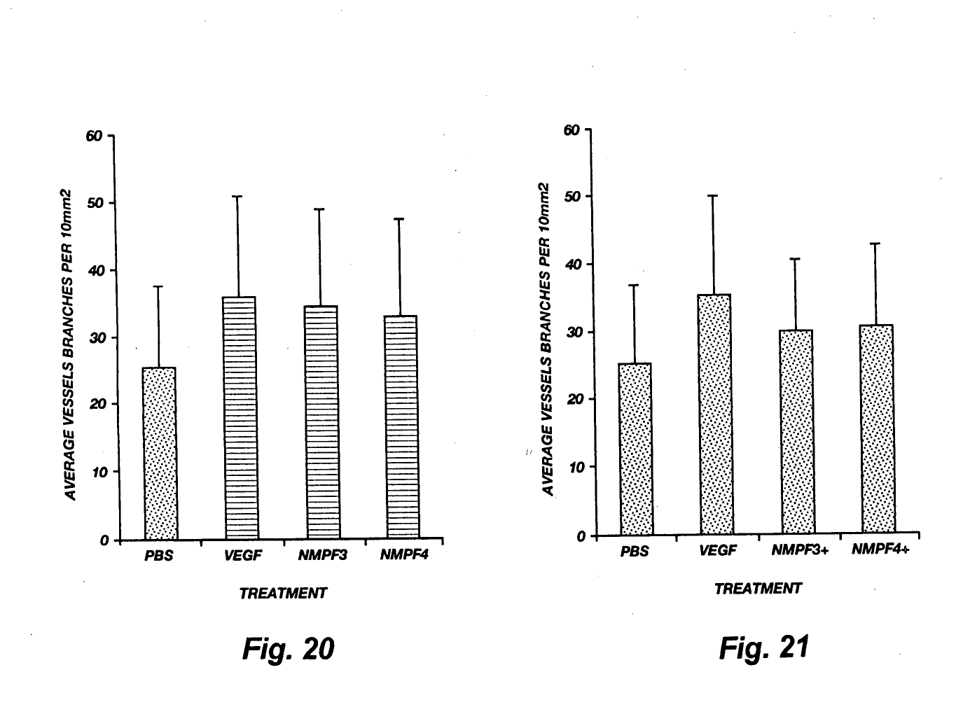

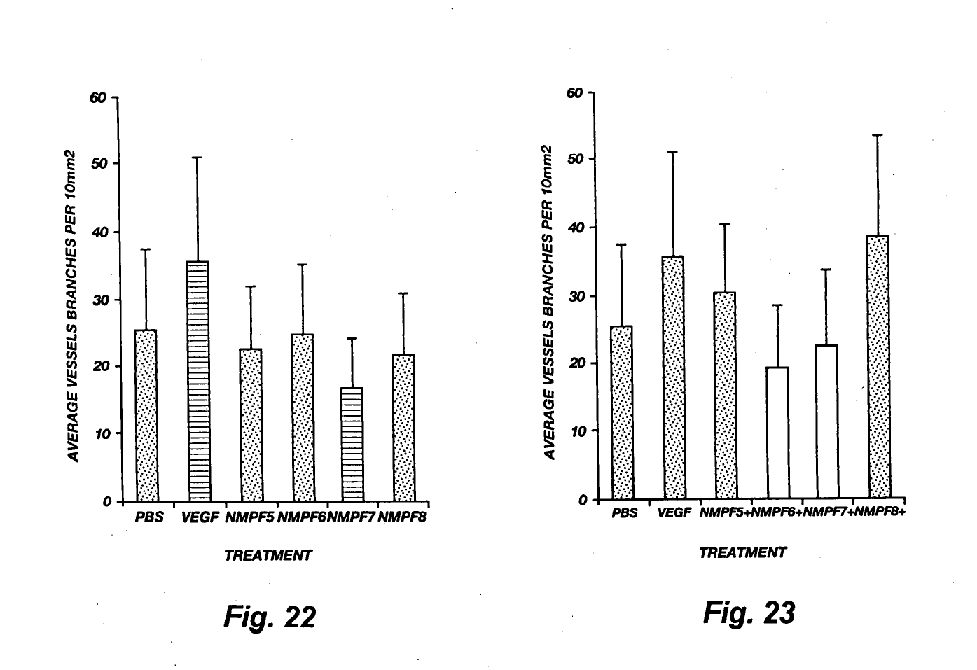

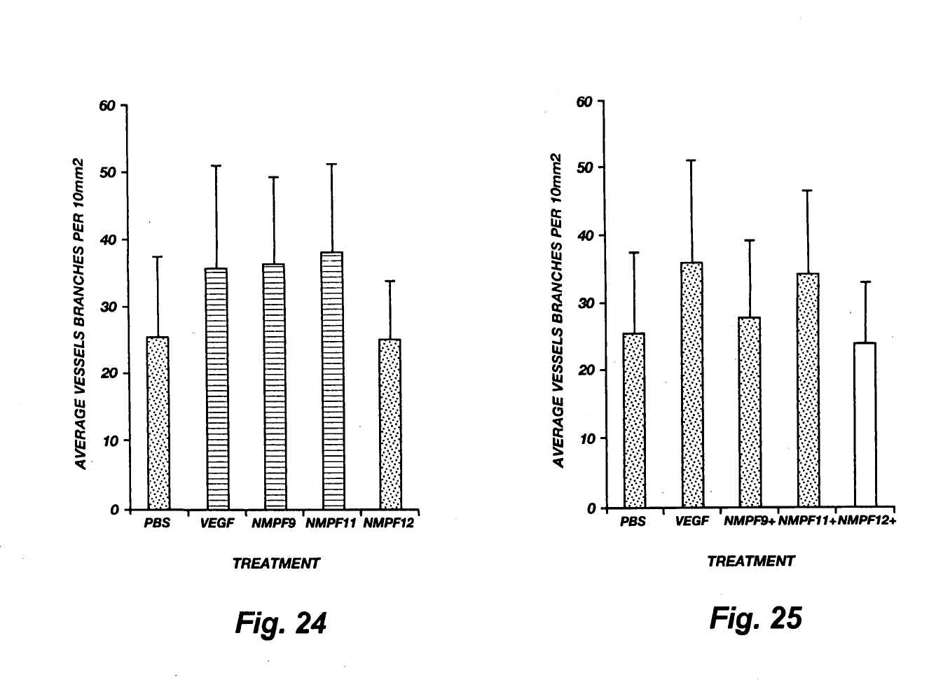

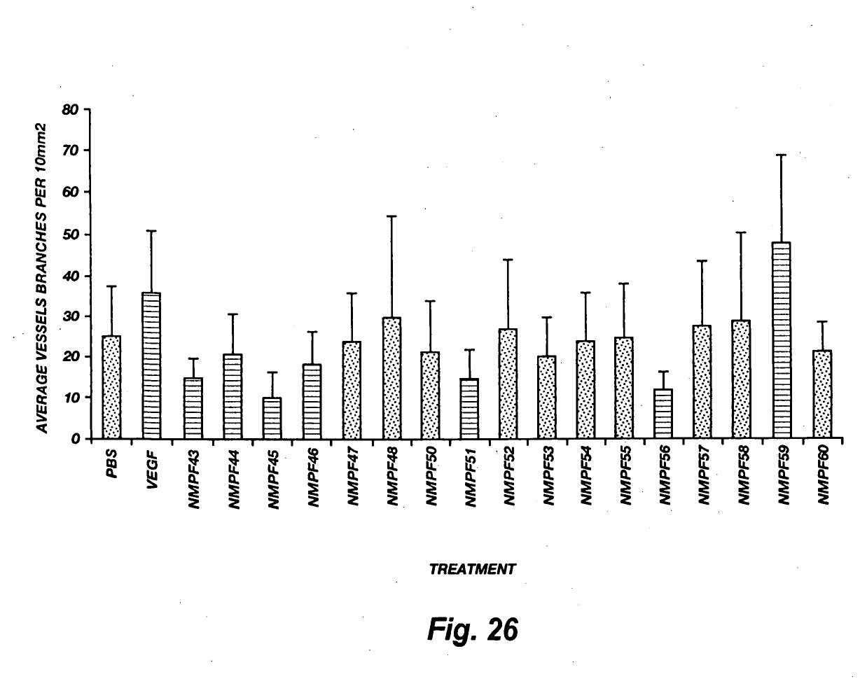

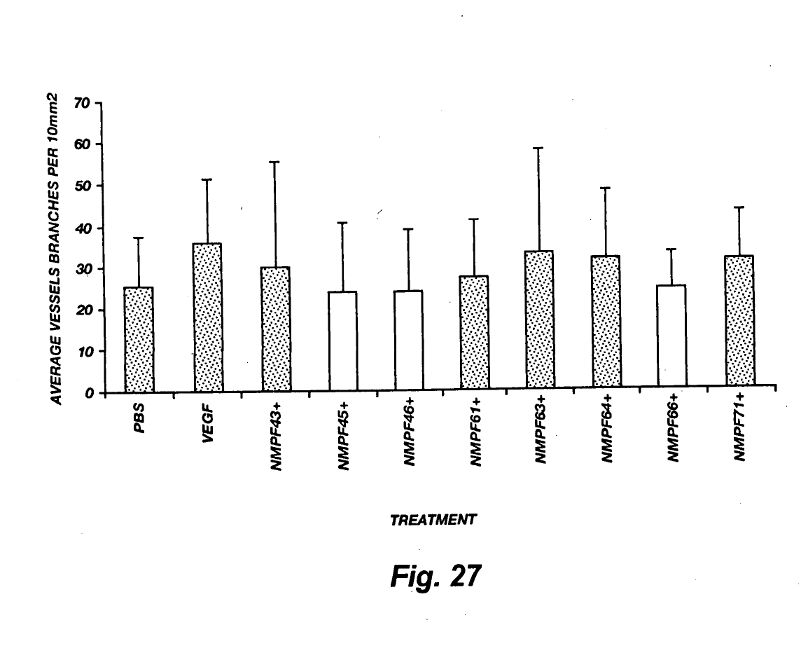

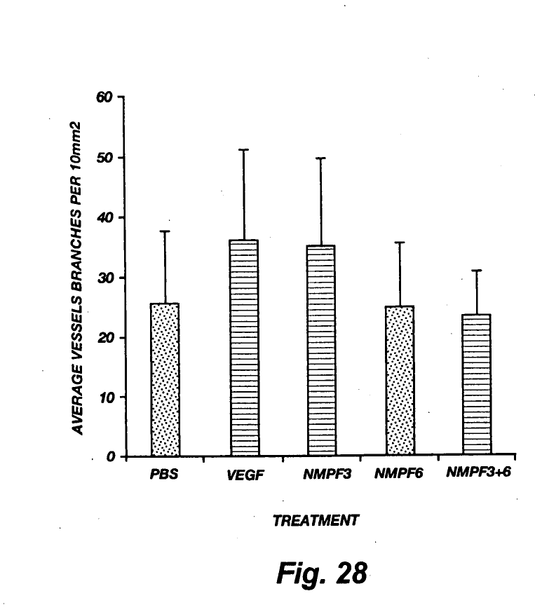

In order to determined the effect of NMPF on angiogenesis and vasculogenesis. we treated fertilized chicken eggs with NMPF or NMPF in combination with VEGF as described in materials and methods section. FIGS. 18 through 28 show that NMPF-3, 4, 9 and 11 promoted angiogenesis (p<0.05), while NMPF VEGF 7, 43, 44, 45, 46, 51 and 56 inhibited angiogenesis (p<0.05). NMPF-6, 7, 12, 45, 46 and 66 were able to inhibit angiogenesis induced by VEGF. Moreover, NMPF-6 itself did not show any effect on angiogenesis, but it inhibited (p<0.05) NMPF-3-induced angiogenesis.

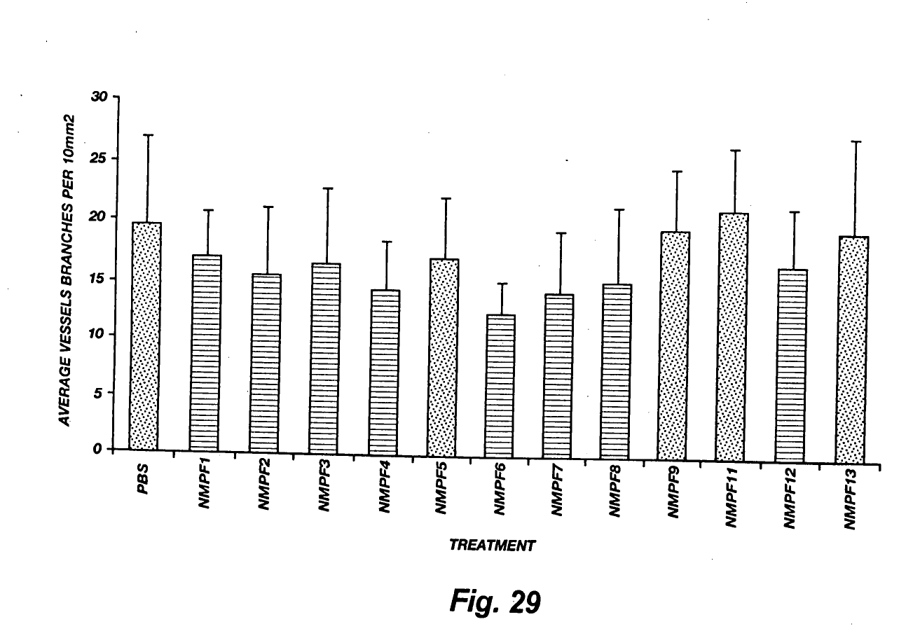

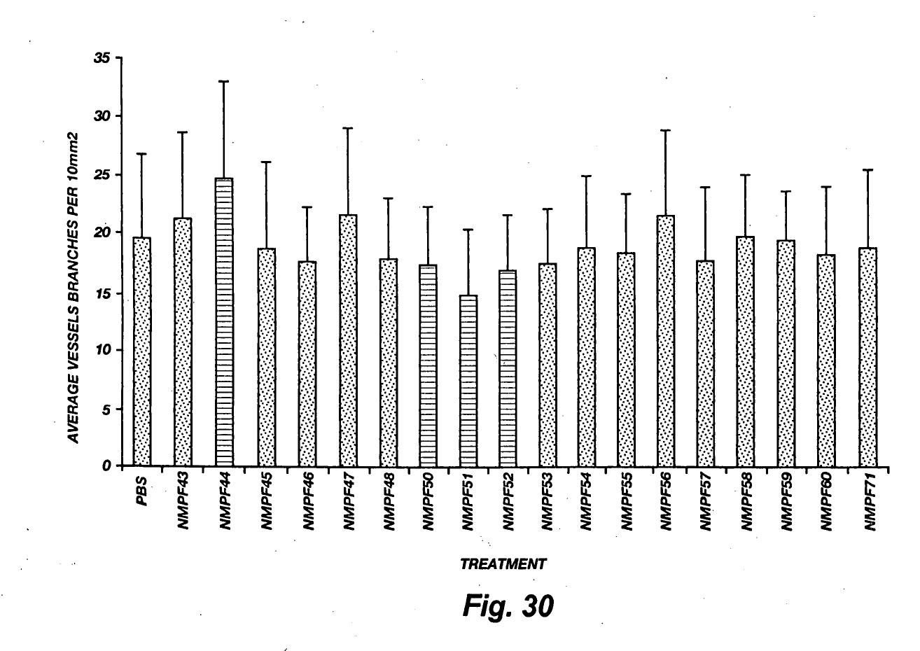

FIGS. 29 and 30 show that NMPF-1, 2, 3, 4, 6, 7, 8, 12, 50, 51, and 52 had vasculogenesis-inhibiting (p<0.05) effect, while only NMPF-44 promoted (p<0.05) vasculogenesis.

### NOD Experiment

Mice. Female NOD mice at the age of 13 to 14 weeks were treated i.p. with PBS (n=6) or NMPF peptides (VLPALPQVVC (SEQ ID NO:20), LQGV (SEQ ID NO:1), GVLPALPQ (SEQ ID NO:33), VLPALP (SEQ ID NO:3), VLPALPQ (SEQ ID NO:29), MTRV (SEQ ID NO:42), LPGCPRGVNPVVS (SEQ ID NO:40), CPRGVNPVVS (SEQ ID NO:50), LPGC (SEQ ID NO:41), MTRVLQGVLPALPQVVC (SEQ ID NO:44), VVCNYRDVRFESIRLPGCPRGVNPVVSYAVA LSCQCAL (SEQ ID NO:35)) (5 mg/kg, n=6) three times a week for two weeks. Every four days, urine was checked for the presence of glucose (Gluketur Test; Boehringer Mannheim, Mannheim, DE). All mice used in these studies were maintained in a pathogen-free facility. They were given free access to food and water. The experiments were approved by the Animal Experiments Committee of the Erasmus University Rotterdam. Diabetes was assessed by measurement of the venous blood glucose level using an Abbott Medisense Precision glucometer. Mice were considered diabetic after two consecutive glucose measurements ≧11 mmol/l (200 mg/dl). Onset of diabetes was dated from the first consecutive reading.

A glucose tolerance test (GTT) was performed at 28 weeks of age in fasted mice (n=5) by injecting 1 g/kg D-glucose intraperitoneally (i.p.). At 0 (fasting), 5, 30 and 60 minutes, blood samples were collected from the tail and tested for glucose content.

### NO Experiment

Cell culture. The RAW 264.7 murine macrophage cell line, obtained from American Type Culture Collection (Manassas, Va., USA), was cultured at 37° C. in 5% CO₂ using DMEM containing 10% fetal calf serum (FCS), 50 U/ml penicillin, 50 μg/ml streptomycin, 0.2 M Na-pyruvate, 2 mM glutamine and 50 μM 2-mercaptoethanol (Bio Whittaker, Europe). The medium was changed every two days.

Nitrite measurements. Nitrite production was measured in the RAW 264.7 macrophage supernatants. The cells (7.5×10⁵/ml) were cultured in 48-well plates in 500 μl of culture medium. The cells were stimulated with LPS (10 microg/ml) and/or NMPF (1 pg/ml, 1 ng/ml, 1 μg/ml) for 24 hours, then the culture media were collected. Nitrite was measured by adding 100 microl of Griess reagent (Sigma) to 100 microl samples of culture medium. The OD₅₄₀ was measured using a microplate reader, and the nitrite concentration was calculated by comparison with the OD₅₄₀ produced using standard solutions of sodium nitrite in the culture medium.

### Results (NOD Experiment)

In order to determine whether NMPF has an effect on the disease development in NOD mice, we tested NMPF on pre-diabetic female NOD mice at the age of 13 to 14 weeks. After only two weeks of treatment (injection of NMPF (5 mg/kg) every other day), glucosuria data of all NOD mice was analyzed. At the end of 17 weeks, profound anti-diabetic effect (mice negative for glucosuria) was observed in different NMPF groups as compared to the PBS group, especially in NMPF groups treated with peptides VLPALPQVVC (SEQ ID NO:20), VLPALP (SEQ ID NO:3), MTRV (SEQ ID NO:42), LPGCPRGVNPVVS (SEQ ID NO:40) and LPGC (SEQ ID NO:41). In addition, impairment of the glucose tolerance test was positively correlated to insulitis but negatively correlated to the number of functional beta cells; also this test showed that NOD mice successfully treated with NMPF were tolerant for glucose as compared to the PBS group. Our results show that PBS-treated NOD mice were all diabetic at the age of 23 weeks. Whereas, NOD mice treated three times a week for two weeks with NMPF showed profound inhibition of diabetes development. The strongest anti-diabetic effects were seen with NMPF-1, 4, 5, 6, 7, 65, 66 and commercial hCG preparation (Pregnyl, Organon, Oss, NL, batch no. 235863). These mice had a low fasting blood glucose level and were tolerant for glucose (data partially shown). However, NMPF-71 showed no effect on the incidence of diabetes, while NMPF-64 and NMPF-11 had a moderate anti-diabetic effect.

### NO Experiment

NO production is a central mediator of the vascular and inflammatory response. Our results show that macrophages (RAW 264.7) stimulated with LPS produce large amounts of NO. However, these cells co-stimulated with most of the NMPF peptides (NMPF peptide 1 to 14, 43 to 66 and 69) even in a very low dose (1 pg/ml) inhibited the production of NO.

### Results (ApoE Experiment)

The invention provides a method and a signaling molecule for the treatment of conditions that are associated with dysfunctional LDL receptors such as apoE and other members of the apolipoprotein family. In particular, use of a signaling molecule comprising GVLPALPQ (SEQ ID NO:33) (NMPF-5) and/or VLPALP (SEQ ID NO:3) (NMPF-6) or a functional analogue or derivative thereof is preferred. Groups of apoE-deficient mice (n=6 per group) were fed a high cholesterol food and given PBS or NMPF every other day intraperitoneally. After 2.5 weeks, body weight was determined as shown in the table below.

| | Average Weight | | |
|---|---|---|---|
| | (g) | SD (g) | p-value |
| ApoE-/- PBS | 31.667 | 1.007 | |
| ApoE-/- NMPF-4 | 31.256 | 1.496 | 0.536 |
| ApoE-/- NMPF-5 | 29.743 | 1.160 | 0.019 |
| Background/PBS | 26.760 | 1.582 | 10⁻⁶ |
| ApoE-/- NMPF-6 | 29.614 | 1.064 | 0.004 |

### Analysis of Different Peptides in Databases

Examples of different databases in which peptides were analyzed are:
Proteomics tools: Similarity searches
BLAST database (ExPasy, NCBI)
SMART (EMBL)
PATTINPROT (PBIL)
Post-translational modification prediction
SignalP (CBS)
Primary structure analysis
HLA Peptide Binding Predictions (BIMAS)
Prediction of MHC type I and II peptide binding (SYFPEITHI)
Amino acid scale representation (Hydrophobicity, other conformational parameters, etc.) (PROTSCALE)
Representations of a protein fragment as a helical wheel (HelixWheel/HelixDraw)

### Results

A non-extensive list of relevant oligopeptides useful for application in a method to identify signaling molecules according to the invention derivable from protein databases

pdb|1DE7|1DE7-A interaction of factor xiii activation peptide with alpha-thrombin LQGV (SEQ ID NO:1), LQGVV (SEQ ID NO:53), LQGVVP (SEQ ID NO:54)

pdb|1DL6|1DL6-A solution structure of human tfiib n-terminal domain LDALP (SEQ ID NO:55)

pdb|1QMH|1QMH-A crystal structure of rna 3′-terminal phosphate cyclase, a ubiquitous enzyme LQTV (SEQ ID NO:56), VLPAL (SEQ ID NO:8), LVLQTVLPAL (SEQ ID NO:57)

pdb|1LYP|1LYP CAP18 (RESIDUES 106-137) IQG, IQGL (SEQ ID NO:58), LPKL (SEQ ID NO:59), LLPKL (SEQ ID NO:60)

pdb|1B9O|1B9O-A human alpha-lactalbumin LPEL (SEQ ID NO:61)

pdb|1GLU|1GLU-A glucocorticoid receptor (dna-binding domain) PARP (SEQ ID NO:62)

pdb|2KIN|2KIN-B kinesin (monomeric) from rattus norvegicus MTRI (SEQ ID NO:63)

pdb|1SMP|1SMP-I MOL_ID: 1; molecule: serratia metallo proteinase; chain: A LQKL (SEQ ID NO:64), LQKLL (SEQ ID NO:65), PEAP (SEQ ID NO:66), LQKLLPEAP (SEQ ID NO:67)

pdb|1ES7|1ES7-B complex between bmp-2 and two bmp receptor ia ectodomains LPQ, PTLP (SEQ ID NO:68), LQPTL (SEQ ID NO:69)

pdb|1BHX|1BHX-F x-ray structure of the complex of human alpha-thrombin with the inhibitor SDZ 229-357 LQV, LQVV (SEQ ID NO:70)

pdb|1VCB|1VCB-A the vhl-elonginc-elonginb structure PELP (SEQ ID NO:71)

pdb|1CQK|1CQK-A crystal structure of the ch3 domain from the mak33 antibody PAAP (SEQ ID NO:72), PAAPQ (SEQ ID NO:73), PAAPQV (SEQ ID NO:74)

pdb|1FCB|1FCB-A flavocytochrome LQG

pdb|1LDC|1LDC-A L-lactate dehydrogenase: cytochrome c oxidoreductase (flavocytochrome B=2=) (E.C.1.1.2.3) mutant with TYR 143 replaced by PHE (Y143F) complexed with pyruvate LQG

pdb|1BFB|1BFB basic fibroblast growth factor complexed with heparin tetramer fragment LPAL (SEQ ID NO:75), PALP (SEQ ID NO:76), PALPE (SEQ ID NO:77)

pdb|1MBF|1MBF mouse c-myb dna-binding domain repeat 1 LPN

pdb|1R2A|1R2A-A the molecular basis for protein kinase A LQG, LTELL (SEQ ID NO:78)

pdb|1CKA|1CKA-B C-CRK (N-terminal SH3 domain) (C-CRKSH3-N) complexed with C3G peptide (PRO-PRO-PRO-ALA-LEU-PRO-PRO-LYS-LYS-ARG (SEQ ID NO:79)) PALP (SEQ ID NO:76)

pdb|1RLQ|1RLQ-R C-SRC (SH3 domain) complexed with the proline-rich ligand RLP2 (RALPPLPRY) (NMR, minimized average structure) LPPL (SEQ ID NO:80), PPLP (SEQ ID NO:81)

pdb|1TNT|1TNT MU transposase (DNA-binding domain) (NMR, 33 structures) LPG, LPGL (SEQ ID NO:82), LPK

pdb|1GJS1|GJS-A solution structure of the albumin binding domain of streptococcal protein G LAAL (SEQ ID NO:83), LAALP (SEQ ID NO:84)

pdb|1GBR|1GBR-B growth factor receptor-bound protein 2 (GRB2, N-terminal SH3 domain) complexed with SOS-A peptide (NMR, 29 structures) LPKL (SEQ ID NO:59), PKLP (SEQ ID NO:85)

pdb|1A78|1A78-A complex of toad ovary galectin with thio-digalactose VLPSIP (SEQ ID NO:86)

pdb|1ISA|1ISA-A IRON(II) superoxide dismutase (E.C.1.15.1.1) LPAL (SEQ ID NO:75), PALP (SEQ ID NO:76)

pdb|1FZV|1FZV-A the crystal structure of human placenta growth factor-1 (PLGF-1), an angiogenic protein at 2.0 A resolution PAVP (SEQ ID NO:13), MLPAVP (SEQ ID NO:87)

pdb|1JLI|1JLI human interleukin 3 (IL-3) mutant with truncation at both N- and C-termini and 14 residue changes, NMR, minimized average LPC, LPCL (SEQ ID NO:88), PCLP (SEQ ID NO:89)

pdb|1HSS|1HSS-A 0.19 alpha-amylase inhibitor from wheat VPALP (SEQ ID NO:90)

pdb|3CRX|3CRX-A CRE Recombinase/DNA complex intermediate I LPA, LPAL (SEQ ID NO:75), PALP (SEQ ID NO:76)

pdb|1PRX|1PRX-A HORF6 a novel human peroxidase enzyme PTIP (SEQ ID NO:91), VLPTIP (SEQ ID NO:92)

pdb|1RCY|1RCY rusticyanin (RC) from Thiobacillus ferrooxidans VLPGFP (SEQ ID NO:93)

pdb|1A3Z|1A3Z reduced Rusticyanin At 1.9 Angstroms PGFP (SEQ ID NO:94), VLPGFP (SEQ ID NO:93)

pdb|1GER|1GER-A glutathione reductase (E.C.1.6.4.2) complexed with FAD LPALP (SEQ ID NO:95), PALP (SEQ ID NO:76)

pdb|1PBW|1PBW-A structure of BCR-homology (BH) domain PALP (SEQ ID NO:76)

pdb|1BBS|1BBS RENIN (E.C.3.4.23.15) MPALP (SEQ ID NO:96)

AI188872 11.3 366 327 18 382 [Homo sapiens]qd27c01.x1 Soares_placenta_{—}8 to 9 weeks_{—}2NbHP8 to 9W H. sapiens cDNA clone IMAGE:1724928 3′ similar to gb:J00117 choriogonadotropin beta chain precursor (human); mRNA sequence; minus strand; translated MXRVLQGVLPALPQVVC (SEQ ID NO:97), MXRV (SEQ ID NO:98), MXR

AI126906 19.8 418 343 1 418 [H. sapiens]qb95f01.x1 Soares_fetal_heart_NbHH19W H. sapiens cDNA clone IMAGE:1707865 3′ similar to gb:J00117 choriogonadotropin Beta chain precursor (HUMAN); mRNA sequence; minus strand; translated ITRVMQGVIPALPQVVC (SEQ ID NO:99)

AI221581 29.1 456 341 23 510 [H. sapiens]qg20a03.x1 Soares_placenta_{—}8 to 9 weeks_{—}2NbHP8 to 9W H. sapiens cDNA clone IMAGE: 1760044 3′ similar to gb:J00117 Choriogonadotropin Beta chain precursor (human); mRNA sequence; minus strand; translated MTRVLQVVLLALPQLV (SEQ ID NO:100)

Mm.42246.3 Mm.42246 101.3 837 304 28 768 GENE=Pck1 PROTSIM=pir:T24168 phosphoenolpyruvate carboxykinase 1,cytosolic; translated KVIQGSLDSLPQAV (SEQ ID NO:101), LDSL (SEQ ID NO:102), LPQ

Mm.22430.1 Mm.22430 209.4 1275 157 75 1535 GENE=Ask-pending PROTSIM=pir:T02633 activator of S phase kinase; translated VLQAILPSAPQ (SEQ ID NO:103), LQA, LQAIL (SEQ ID NO:104), PSAP (SEQ ID NO:105), LPS

Hs.63758.4 Hs.63758 93.8 3092 1210 51 2719 GENE=TFR2 PROTSIM=pir:T30154 transferrin receptor 2; translated KVLQGRLPAVAQAV (SEQ ID NO:106), LQG, LPA, LPAV (SEQ ID NO:107)

Mm.129320.2 Mm.129320 173.0 3220 571 55 2769 GENE=PROTSIM=pir:T16409 Sequence 8 from Patent WO9950284; translated LVQKVVPMLPRLLC (SEQ ID NO:108), LVQ, LPRL (SEQ ID NO:109), PMLP (SEQ ID NO:110)

Mm.22430.1 Mm.22430 209.4 1275 157 75 1535 GENE=Ask-pending PROTSIM=pir:T02633 activator of S phase kinase; translated VLQAILPSAPQ (SEQ ID NO:103), LQA, LQAIL (SEQ ID NO:104), PSAP (SEQ ID NO:105), PSAPQ (SEQ ID NO:111)

P20155 IAC2_HUMAN Acrosin-trypsin inhibitor II precursor (HUSI-II) [SPINK2] [H. sapiens] LPGCPRHFNPV (SEQ ID NO:112), LPG, LPGC (SEQ ID NO:41)

Rn.2337.1 Rn.2337 113.0 322 104 1 327 GENE=PROTSIM=PRF:1402234A Rat pancreatic secretory trypsin inhibitor type II (PSTI-II) mRNA, complete cds; minus strand; translated LVGCPRDYDPV (SEQ ID NO:113), LVG, LVGC (SEQ ID NO:114)

Hs.297775.1 Hs.297775 43.8 1167 753 31 1291 GENE=PROTSIM=sp:O00268 ESTs, Weakly similar to T2D3_HUMAN transcription initiation factor TFIID 135 KDA SUBUNIT [H. sapiens]; minus strand; translated PGCPRG (SEQ ID NO:115), PGCP (SEQ ID NO:10)

Mm.1359.1 Mm.1359 PROTSTM=pir.A39743 urokinase plasminogen activator receptor LPGCP (SEQ ID NO:116), PGCP (SEQ ID NO:10), LPG, LPGC (SEQ ID NO:40)

sptrembl|O56177|O56177 envelope glycoprotein VLPAAP (SEQ ID NO:117), PAAP (SEQ ID NO:72)

sptrembl|Q9W234|Q9W234 CG13509 PROTEIN.//:trembl|AE003458| AE003458_{—}7 gene: “CG13509”; Drosophila melanogaster genomic scaffold LAGTIPATP (SEQ ID NO:118), LAG, PATP (SEQ ID NO:119)

swiss|P81272|NS2B_HUMAN NITRIC-OXIDE SYNTHASE IIB (EC 1.14.13.39) (NOS, TYPE II B) (NOSIIB) (FRAGMENTS) GVLPAVP (SEQ ID NO:11), LPA, VLPAVP (SEQ ID NO:12), PAVP (SEQ ID NO:13)

sptrembl|O30137|O30137 HYPOTHETICAL 17.2 KDA GVLPALP (SEQ ID NO:32), PALP (SEQ ID NO:76), LPAL (SEQ ID NO:75)

sptrembl|Q9IYZ3|Q9IYZ3 DNA POLYMERASE GLLPCLP (SEQ ID NO:120), LPC, LPCL (SEQ ID NO:88), PCLP (SEQ ID NO:89)

sptremb|Q9PVW5|Q9PVW5 NUCLEAR PROTEIN NP220 PGAP (SEQ ID NO:121), LPQRPRGPNP (SEQ ID NO:122), LPQ, PRGP (SEQ ID NO:123), PNP

Hs.303116.2 PROTSIM=pir; T33097 stromal cell-derived factor 2-like1; translated GCPR (SEQ ID NO:124)

pdb|1DU3|1DU3-A CRYSTAL STRUCTURE OF TRAIL-SDR5GCPRGM (SEQ ID NO:125)

pdb|1D0G|1DOG-R CRYSTAL STRUCTURE OF DEATH RECEPTOR 5 (DR5) BOUND TO APO2L/TRAIL GCPRGM (SEQ ID NO:125)

pdb|1BIO|1BIO human complement factor d in complex with isatoic anhydride inhibitor LQHV (SEQ ID NO:126)

pdb|4NOS|4NOS-A human inducible nitric oxide synthase with inhibitor FPGC (SEQ ID NO:9), PGCP (SEQ ID NO:10)

pdb|1FL7|1FL7-B human follicle stimulating hormone PARP (SEQ ID NO:62), VPGC (SEQ ID NO:127)

pdb|1HR6|1HR6-A yeast mitochondrial processing peptidase CPRG (SEQ ID NO:128), LKGC (SEQ ID NO:129)

pdb|1BFA|1BFA recombinant bifunctional hageman factor/amylase inhibitor from PPGP (SEQ ID NO:130), LPGCPREV (SEQ ID NO:131), LPGC (SEQ ID NO:41), PGCP (SEQ ID NO:10), CPRE (SEQ ID NO:132)

swissnew|P01229|LSHB_HUMAN Lutropin β chain precursor MMRVLQAVLPPLPQVVC (SEQ ID NO:133), MMR, MMRV (SEQ ID NO:134), LQA, LQAV (SEQ ID NO:52), VLPPLP (SEQ ID NO:135), PPLP (SEQ ID NO:81), QVVC (SEQ ID NO:43), VVC, VLPPLPQ (SEQ ID NO:136), AVLPPLP (SEQ ID NO:137), AVLPPLPQ (SEQ ID NO:138)

swissnew|P07434|CGHB_PAPAN Choriogonadotropin beta chain precursor MMRVLQAVLPPVPQVVC (SEQ ID NO:139), MMR, MMRV (SEQ ID NO:134), LQA, LQAG (SEQ ID NO:140), VLPPVP (SEQ ID NO:141), VLPPVPQ (SEQ ID NO:142), QVVC (SEQ ID NO:43), VVC, AVLPPVP (SEQ ID NO:143), AVLPPVPQ (SEQ ID NO:144)

swissnew|Q28376TSHB_HORSE Thyrotropin beta chain precursor MTRD (SEQ ID NO:145), LPK, QDVC (SEQ ID NO:146), DVC, IPGC (SEQ ID NO:147), PGCP (SEQ ID NO:10)

swissnew|P95180|NUOB_MYCTU NADH dehydrogenase I chain B LPGC (SEQ ID NO:41), PGCP (SEQ ID NO:10)

sptrembl|Q9Z284|Q9Z284 NEUTROPHIL ELASTASE PALP (SEQ ID NO:76), PALPS (SEQ ID NO:148)

sptrembl|Q9UCG8|Q9UCG8 urinary gonadotrophin peptide (fragment). LPGGPR (SEQ ID NO:149), LPG, LPGG (SEQ ID NO:150), GGPR (SEQ ID NO:151)

XP_{—}028754 growth hormone releasing hormone [H. sapiens] LQRG (SEQ ID NO:152), LQRGV (SEQ ID NO:153), LGQL (SEQ ID NO:154)

SignalP (CBS)

SignalP predictions: (for example)

MTRVLQGVLPALP (SEQ ID NO:155)

QVVC (SEQ ID NO:43)

HLA Peptide Binding Predictions (BIMAS)

(For example)

Half time of
dissociation

| | | | |
|---|---|---|---|
| HLA molecule type I (A_0201): | VLQGVLPAL | (SEQ ID NO:156) | (84) |
| | GVLPALPQV | (SEQ ID NO:157) | (51) |
| | VLPALPQVV | (SEQ ID NO:158) | (48) |
| | RLPGCPRGV | (SEQ ID NO:159) | (14) |
| | TMTRVLQGV | (SEQ ID NO:160) | (115) |

scores

| | | | |
|---|---|---|---|
| MHC II (H2-Ak 15-mers) | C P T M T R V L Q G V L P A L | (SEQ ID NO:161) | 14 |
| | P G C P R G V N P V V S Y A V | (SEQ ID NO:162) | 14 |
| HLA-DRB1*010115-mers | P R G V N P V V S Y A V A L S | (SEQ ID NO:163) | 29 |
| | T R V L Q G V L P A L P Q V V | (SEQ ID NO:164) | 28 |
| | L Q G V L P A L P Q V V C N Y | (SEQ ID NO:165) | 22 |
| HLA-DRB1*0301 (DR 17) | C P T M T R V L Q G V L P A L | (SEQ ID NO:161) | 26 |
| 15-mers | M T R V L Q G V L P A L P Q V | (SEQ ID NO:166) | 21 |
| | S I R L P G C P R G V N P V V | (SEQ ID NO:167) | 17 |

TABLE 1
Results of shock experiments in mice

% SURVIVAL IN
TIME (HOURS)

| | | | |
|---|---|---|---|
| 0 | 16 | 40 | 72 |

TEST SUBSTANCE

| | | | | |
|---|---|---|---|---|
| PBS | 100 | 100 | 67 | 17 |
| PG23 | 100 | 100 | 100 | 100 |
| PG25 | 100 | 83 | 83 | 83 |

| | |
|---|---|
| PEPTIDE | |
| NMPF | SEQUENCE |

| | | | | | | |
|---|---|---|---|---|---|---|
| 1 | VLPALPQVVC | (SEQ ID NO:28) | 100 | 100 | 50 | 17 |
| 2 | LQGVLPALPQ | (SEQ ID NO:49) | 100 | 67 | 0 | 0 |
| 3 | LQG | | 100 | 83 | 20 | 17 |
| 4 | LQGV | (SEQ ID NO:1) | 100 | 100 | 100 | 100 |
| 5 | GVLPALPQ | (SEQ ID NO:33) | 100 | 100 | 80 | 17 |
| 6 | VLPALP | (SEQ ID NO:3) | 100 | 100 | 100 | 100 |
| 7 | VLPALPQ | (SEQ ID NO:29) | 100 | 83 | 0 | 0 |
| 8 | GVLPALP | (SEQ ID NO:32) | 100 | 100 | 83 | 67 |
| 9 | VVC | | 100 | 100 | 50 | 50 |
| 11 | MTRV | (SEQ ID NO:42) | 100 | 100 | 67 | 50 |
| 12 | MTR | | 100 | 100 | 67 | 50 |
| 13 | LQGVLPALPQVVC | (SEQ ID NO:34) | 100 | 100 | 100 | 100 |
| 14 | (CYCLIC) | (SEQ ID NO:34) | 100 | 83 | 83 | 83 |
| 64 | LPGCPRGVNPVVS | (SEQ ID NO:40) | 100 | 100 | 100 | 100 |
| 66 | LPGC | (SEQ ID NO:41) | 100 | 100 | 100 | 100 |

TABLE 2
Additional results of shock experiments

| | |
|---|---|
| NMPF SEQUENCE ID: | ANTI-SHOCK EFFECT |

| | | |
|---|---|---|
| LQGV | (SEQ ID NO:1) | +++ |
| AQGV | (SEQ ID NO:2) | +++ |
| LQGA | (SEQ ID NO:19) | +++ |
| VLPALP | (SEQ ID NO:3) | +++ |
| ALPALP | (SEQ ID NO:21) | ++ |
| VAPALP | (SEQ ID NO:22) | ++ |
| ALPALPQ | (SEQ ID NO:23) | ++ |
| VLPAAPQ | (SEQ ID NO:24) | ++ |
| VLPALAQ | (SEQ ID NO:25) | +++ |

SHOCK ACCELERATING EFFECT

| | | |
|---|---|---|
| LAGV | (SEQ ID NO:26) | +++ |
| LQAV | (SEQ ID NO:52) | +++ |
| VLAALP | (SEQ ID NO:27) | +++ |
| VLPAAP | (SEQ ID NO:117) | +++ |
| VLPALA | (SEQ ID NO:28) | +++ |
| VLPALPQ | (SEQ ID NO:29) | +++ |
| VLAALPQ | (SEQ ID NO:30) | +++ |
| VLPALPA | (SEQ ID NO:31) | +++ |

TABLE 3
Further additional results of shock experiments

% SURVIVAL IN TIME (HOURS)

| | | | | |
|---|---|---|---|---|
| | Tx | | Tx | |
| NMPF PEPTIDES | 0 | 14 | 24 | 48 |

| | | | | |
|---|---|---|---|---|
| PBS | 100 | 100 | 100 | 0 |
| NMPF-3 | 100 | 100 | 100 | 0 |
| NMPF-5 | 100 | 100 | 100 | 100 |
| NMPF-7 | 100 | 100 | 100 | 67 |
| NMPF-8 | 100 | 100 | 100 | 100 |
| NMPF-9 | 100 | 100 | 100 | 100 |
| NMPF-11 | 100 | 100 | 100 | 100 |
| NMPF-12 | 100 | 100 | 100 | 100 |
| NMPF-43 | 100 | 100 | 100 | 100 |
| NMPF-45 | 100 | 100 | 100 | 100 |
| NMPF-46 | 100 | 100 | 100 | 100 |
| NMPF-50 | 100 | 100 | 100 | 100 |
| NMPF-53 | 100 | 100 | 100 | 100 |
| NMPF-58 | 100 | 100 | 100 | 100 |
| NMPF-60 | 100 | 100 | 100 | 100 |

TABLE 4
Further additional results

| | | | SCORES | |
|---|---|---|---|---|
| | Tx | SICKNESS | Tx | |
| NMPF PEPTIDES | 0 | 14 | 24 | 48 |
| PBS | 0, 0, 0, 0, 0, 0 | 5, 5, 5, 5, 4, 4 | 5, 5, 5, 5, 5, 5 | †††††† |
| NMPF-3 | 0, 0, 0, 0, 0, 0 | 3, 3, 3, 3, 3, 4 | 4, 4, 4, 4, 4, 4 | †††††† |
| NMPF-5 | 0, 0, 0, 0, 0, 0 | 5, 5, 5, 5, 5, 5 | 5, 5, 5, 5, 5, 5 | 2, 2, 2, 2, 2, 2 |
| NMPF-7 | 0, 0, 0, 0, 0, 0 | 1, 1, 4, 4, 4, 4 | 5, 5, 5, 5, 5, 5 | 2, 2, 2, 2, †† |
| NMPF-8 | 0, 0, 0, 0, 0, 0 | 3, 3, 5, 5, 5, 5 | 5, 5, 5, 5, 5, 5 | 2, 2, 4, 4, 4, 5 |
| NMPF-9 | 0, 0, 0, 0, 0, 0 | 3, 3, 4, 4, 5, 5 | 2, 2, 2, 2, 2, 2 | 1, 1, 2, 2, 2, 2 |
| NMPF-11 | 0, 0, 0, 0, 0, 0 | 1, 1, 3, 3, 4, 4, | 2, 2, 2, 2, 4, 4 | 1, 1, 1, 1, 1, 1 |
| NMPF-12 | 0, 0, 0, 0, 0, 0 | 1, 1, 1, 1, 3, 3 | 1, 1, 1, 1, 1, 1 | 1, 1, 1, 1, 1, 1 |
| NMPF-43 | 0, 0, 0, 0, 0, 0 | 1, 1, 4, 4, 4, 4 | 1, 1, 1, 1, 3, 3 | 2, 2, 2, 2, 2, 2 |
| NMPF-45 | 0, 0, 0, 0, 0, 0 | 5, 5, 5, 5, 4, 4 | 3, 3, 4, 4, 5, 5 | 2, 2, 4, 4, 5, 5 |
| NMPF-46 | 0, 0, 0, 0, 0, 0 | 1, 1, 2, 2, 3, 3 | 1, 1, 2, 2, 2, 2 | 1, 1, 1, 1, 1, 1 |
| NMPF-50 | 0, 0, 0, 0, 0, 0 | 1, 1, 1, 1, 3, 3 | 2, 2, 2, 2, 3, 3 | 1, 1, 1, 1, 1, 1 |
| NMPF-53 | 0, 0, 0, 0, 0, 0 | 5, 5, 5, 5, 5, 5 | 5, 5, 5, 5, 5, 5 | 1, 1, 2, 2, 2, 2 |
| NMPF-58 | 0, 0, 0, 0, 0, 0 | 5, 5, 5, 5, 3, 3 | 5, 5, 5, 5, 3, 3 | 1, 1, 1, 1, 1, 1 |
| NMPF-60 | 0, 0, 0, 0, 0, 0 | 1, 1, 4, 4, 2, 2 | 2, 2, 2, 2, 4, 4 | 1, 1, 1, 1, 1, 1 |

TABLE 5
Summary of results of the various peptides in the various experiments.

| | | | | | | |
|---|---|---|---|---|---|---|
| ID | SEQUENCE | SEPSIS | ANGIOGENESIS | CAO | DC | NOD |

| | | | | | | |
|---|---|---|---|---|---|---|
| NMPF-1 | VLPALPQVVC (SEQ ID NO:20) | −+ | | + | + | |
| NMPF-2 | LQGVLPALPQ (SEQ ID NO:49) | −+ | | | + | |
| NMPF-3 | LQG | −+ | + | + | + | |
| NMPF-4 | LQGV (SEQ ID NO:1) | + | + | + | + | |
| NMPF-5 | GVLPALPQ (SEQ ID NO:33) | −+ | | | + | |
| NMPF-6 | VLPALP (SEQ ID NO:3) | + | + | + | + | |
| NMPF-7 | VLPALPQ (SEQ ID NO:29) | + | + | | + | |
| NMPF-8 | GVLPALP (SEQ ID NO:32) | −+ | | | + | |
| NMPF-9 | VVC | + | + | | + | |
| NMPF-10 | QVVC (SEQ ID NO:43) | | | | | |
| NMPF-11 | MTRV (SEQ ID NO:42) | + | + | | + | + |
| NMPF-12 | MTR | −+ | + | | + | |
| NMPF-13 | LQGVLPALPQVVC (SEQ ID NO:34) | + | | | + | |
| NMPF-14 | cyclic-LQGVLPALPQVVC (SEQ ID NO:34) | + | | | | |
| NMPF-43 | AQG | + | + | | + | |
| NMPF-44 | LAG | | + | | | |
| NMPF-45 | LQA | + | + | | | |
| NMPF-46 | AQGV (SEQ ID NO:2) | + | + | | + | |
| NMPF-47 | LAGV (SEQ ID NQ:26) | −+ | | + | + | |
| NMPF-48 | LQAV (SEQ ID NO:52) | | | | | |
| NMPF-49 | LQGA (SEQ ID NO:19) | + | | | | |
| NMPF-50 | ALPALP (SEQ ID NO:21) | + | | | + | |
| NMPF-51 | VAPALP (SEQ ID NO:22) | + | + | | | |
| NMPF-52 | VLAALP (SEQ ID NO:27) | | | | | |
| NMPF-53 | VLPAAP (SEQ ID NO:117) | + | | | + | |
| NMPF-54 | VLPALA (SEQ ID NO:28) | | | | | |
| NMPF-55 | ALPALPQ (SEQ ID NO:23) | + | | | | |
| NMPF-56 | VAPALPQ (SEQ ID NO:173) | | + | | | |
| NMPF-57 | VLAALPQ (SEQ ID NO:30) | | | | | |
| NMPF-58 | VLPAAPQ (SEQ ID NO:24) | + | | | + | |
| NMPF-59 | VLPALAQ (SEQ ID NO:25) | + | + | | | |
| NMPF-60 | VLPALPA (SEQ ID NO:31) | + | | | + | |
| NMPF-61 | VVCNYRDVRFESIRLPGCPRGVNPVVSYAVALSCQCAL | −+ | | + | | |
| | (SEQ 1D NO:35) | | | | | |
| NMPF-62 | VVCNYRDVRFESIRLPGCPRGVNPVVSYAVALSCQ | | | | | |
| | (SEQ ID NO:169) | | | | | |
| NMPF-63 | SIRLPGCPRGVNPVVS (SEQ ID NO:39) | −+ | | | | |
| NMPF-64 | LPGCPRGVNPVVS (SEQ ID NO:40) | | | + | | |
| NMPF-65 | CPRGVNPVVS (SEQ ID NO:50) | | | | | |
| NMPF-66 | LPGC (SEQ ID NO:41) | + | + | + | | |
| NMPF-67 | CPRGVNP (SEQ ID NO:170) | | | | | |
| NMPF-68 | PGCP (SEQ ID NO:10) | −+ | | | | |
| NMPF-69 | RPRCRPINATLAVEKEGCPVCITVNTTICAGYCPT | | | | | |
| | (SEQ ID NO:45) | | | | | |
| NMPF-70 | MTRVLQGVLPALPQ (SEQ ID NO:171) | −+ | | | | |
| NMPF-71 | MTRVLPGVLPALPQVVC (SEQ ID NO:174) | −+ | | | | |
| NMPF-74 | CALCRRSTTDCGGPKDHPLTC (SEQ ID NO:46) | | | | | |
| NMPF-75 | SKAPPPSLPSPSRLPGPC (SEQ ID NO:172) | | | | | |
| NMPF-76 | TCDDPRFQDSSSSKAPPPSLPSPSRLPGPSDTPILPQ | | | | | |
| | (SEQ ID NO:48) | | | | | |

+ = effects; −+ = variable effect; no entry is no effect or not yet tested when table was assembled

TABLE 6
MODULATION OF NO AND/OR TNF-α

| | | | | |
|---|---|---|---|---|
| ID | SEQUENCE | TNF-A | NO | TNF-A and NO |

| | | | | |
|---|---|---|---|---|
| NMPF-1 | VLPALPQVVC (SEQ ID NO:20) | ++ | ++++ | ++++ |
| NMPF-2 | LQGVLPALPQ (SEQ ID NO:49) | −+ | ++++ | ++++ |
| NMPF-3 | LQG | + | ++++ | ++++ |
| NMPF-4 | LQGV (SEQ ID NO:1) | ++++ | ++++ | +++++++ |
| NMPF-5 | GVLPALPQ (SEQ ID NO:33) | ++++ | ++++ | +++++++ |
| NMPF-6 | VLPALP (SEQ ID NO:3) | ++++ | ++++ | +++++++ |
| NMPF-7 | VLPALPQ (SEQ ID NO:29) | ++++ | ++++ | +++++++ |
| NMPF-8 | GVLPALP (SEQ ID NO:32) | ++++ | ++++ | +++++++ |
| NMPF-9 | VVC | ++++ | ++++ | +++++++ |
| NMPF-10 | QVVC (SEQ ID NO:43) | ++++ | +++ | ++++ |
| NMPF-11 | MTRV (SEQ ID NO:42) | ++++ | ++++ | ++++ |
| NMPF-12 | MTR | ++++ | ++++ | ++++ |
| NMPF-13 | LQGVLPALPQVVC (SEQ ID NO:34) | ++ | ++++ | ++++ |
| NMPF-14 | cyclic-LQGVLPALPQVVC (SEQ ID NO:34) | ++ | ++++ | ++++ |
| NMPF-43 | AQG | ++++ | ++++ | +++++++ |
| NMPF-44 | LAG | −+ | ++++ | ++++ |
| NMPF-45 | LQA | −+ | ++++ | +++++++ |
| NMPF-46 | AQGV (SEQ ID NO:2) | ++++ | ++++ | +++++++ |
| NMPF-47 | LAGV (SEQ ID NO:26) | ++ | ++++ | ++++ |
| NMPF-48 | LQAV (SEQ ID NO:52) | ++ | ++++ | ++++ |
| NMPF-49 | LQGA (SEQ ID NO:19) | ++ | ++++ | ++++ |
| NMPF-50 | ALPALP (SEQ ID NO:21) | ++++ | ++++ | +++++++ |
| NMPF-51 | VAPALP (SEQ ID NO:22) | + | +++ | ++++ |
| NMPF-52 | VLAALP (SEQ ID NO:27) | ++ | ++++ | ++++ |
| NMPF-53 | VLPAAP (SEQ ID NO:117) | ++++ | ++++ | +++++++ |
| NMPF-54 | VLPALA (SEQ ID NO:28) | + | ++++ | ++++ |
| NMPF-55 | ALPALPQ (SEQ ID NO:23) | + | ++++ | ++++ |
| NMPF-56 | VAPALPQ (SEQ ID NO:173) | −+ | ++++ | ++++ |
| NMPF-57 | VLAALPQ (SEQ ID NO:30) | + | ++++ | ++++ |
| NMPF-58 | VLPAAPQ (SEQ ID NO:24) | ++++ | ++++ | +++++++ |
| NMPF-59 | VLPALAQ (SEQ ID NO:25) | ++ | ++++ | ++++ |
| NMPF-60 | VLPALPA (SEQ ID NO:31) | ++++ | ++++ | +++++++ |
| NMPF-61 | (SEQ ID NO:35) | −+ | ++++ | ++++ |
| NMPF-62 | (SEQ ID NO:169) | −+ | +++ | |
| NMPF-63 | SIRLPGCPRGVNPVVS (SEQ ID NO:39) | −+ | ++ | ++ |
| NMPF-64 | LPGCPRGVNPVVS (SEQ ID NO:40) | ++ | ++++ | ++++ |
| NMPF-65 | CPRGVNPVVS (SEQ ID NO:50) | ++ | +++ | +++ |
| NMPF-66 | LPGC (SEQ ID NO:41) | +++ | ++ | +++ |
| NMPF-67 | CPRGVNP (SEQ ID NO:170) | −+ | + | + |
| NMPF-68 | PGCP (SEQ ID NO:10) | + | + | +++ |
| NMPF-69 | (SEQ ID NO:45) | −+ | ++ | ++ |
| NMPF-70 | MTRVLQGVLPALPQ (SEQ ID NO:171) | −+ | + | + |
| NMPF-71 | MTRVLPGVLPALPQVVC (SEQ ID NO:174) | −+ | −+ | −+ |
| NMPF-74 | CALCRRSTTDCGGPKDHPLTC (SEQ ID NO:46) | −+ | ++ | + |
| NMPF-75 | SKAPPPSLPSPSRLPGPS (SEQ ID NO:172) | + | ++ | ++ |
| NMPF-76 | (SEQ ID NO:48) | + | + | + |
| NMPF-78 | CRRSTTDCGGPKDHPLTC (SEQ ID NO:47) | + | + | + |

from −+to +++++++indicates from barely active to very active in modulating

### Monkey Experiment (Efficacy of NMPF)

Here, a mixture 1:1:1 of SEQ ID NO:1, SEQ ID NO:2 and SEQ ID NO:3, administered in a gram-(−)-induced rhesus monkey sepsis model for prevention of septic shock.

Overwhelming inflammatory and immune responses are essential features of septic shock and play a central part in the pathogenesis of tissue damage, multiple organ failure, and death induced by sepsis. Cytokines, especially tumor necrosis factor (TNF)-α interleukin (IL)-1β, and macrophage migration inhibitory factor (MIF), have been shown to be critical mediators of septic shock. Yet, traditional anti-TNF and anti-IL-1 therapies have not demonstrated much benefit for patients with severe sepsis. We have designed peptides that block completely LPS-induced septic shock in mice, even when treatment with these peptides is started up to 24 hours after LPS injection. These peptides are also able to inhibit the production of MIF. This finding provides the possibility of therapeutic use of these peptides for the treatment of patients suffering from septic shock. Since primates are evolutionary more closer to humans, we tested these peptides for their safety and effectiveness in a primate system.

### Experimental Design

| | EXPERIMENTAL | | |
|---|---|---|---|
| | TREATMENT | | |
| | (independent variable, | BIO- | |
| | e.g., placebo-treated | TECH- | NUM- |
| GROUP | control group) | NIQUES | BER |
| animal | i.v. infusion of a lethal | Live E. coli | N = 1 |
| I | dose of live | infusion | |
| | Escherichia coli | Blood sampling | |
| | (10E10 CFU/kg) + antibiotics + | No recovery | |
| | placebo treated | (section) | |
| animal | i.v. infusion of a lethal | Live E. coli | N = 1 |
| II | dose of live | infusion | |
| | Escherichia coli | Blood sampling | |
| | (10E10 CFU/kg) + antibiotics + | No recovery | |
| | oligopeptide (5 mg/kg of each | (section) | |
| | of 3 peptides) | | |

Only naive monkeys were used in this pre-clinical study to exclude any interaction with previous treatments. The animals were sedated with ketamine hydrochloride. Animals were intubated orally and allowed to breathe freely. The animals were kept anesthetized with O₂/N₂O/isofluorane. The animals received atropin as pre-medication for O₂/N₂O/isofluorane anesthesia. A level of surgical anesthesia was maintained during the two-hour infusion of E. coli and for six hours following E. coli challenge, after which the endotracheal tubes were removed and the animals were euthanized. Before bacteria were induced, a one-hour pre-infusion monitoring of heart-rate and blood pressure was performed.

Two rhesus monkeys were infused with a 10¹⁰ CFU per kg of the Gram negative bacterium E. coli to induce a fatal septic shock. One monkey received placebo-treatment and was sacrificed within seven hours after infusion of the bacteria without recovery from the anesthesia. The second monkey received treatment with test compound and was sacrificed at the same time point.

In a limited dose-titration experiment performed with the same bacterium strain in 1991, the used dose proved to induce fatal shock within eight hours. In recent experiments, a three-fold lower dose was used inducing clear clinical and pathomorphological signs of septic shock without fatal outcome.

The monkeys were kept anaesthetized throughout the observation period and sacrificed seven hours after the start of the bacterium infusion for pathological examination. The animals underwent a gross necropsy in which the abdominal and thoracic cavities were opened and internal organs examined in situ.

Full Description of the Experiment with Three Rhesus Monkeys

The study was conducted in rhesus monkeys (Maccaca mulatta). Only experimentally naive monkeys were used in the study to exclude any interaction with previous treatments. Prior to the experiment, the state of health of the animals was assessed physically by a veterinarian. All animals had been declared to be in good health and were free of pathogenic ecto- and endoparasites and common bacteriological infections: Yersinia pestis, Yersinia enterocolitica, Yersinia pseudotuberculosis, Shigella, Aeromonas hydrophilia, pathogenic Campylobacter species and Salmonella.

Reagents. The Escherichia coli strain was purchased from ATCC (E. coli; 086a: K61 serotype, ATCC 33985). In a control experiment, the strain proved equally susceptible to bactericidal factors in human and rhesus monkey serum. Prior to the experiment, a fresh culture was set-up; the E. coli strain was cultured for one day, harvested and washed thoroughly to remove free endotoxin. Prior to infusion into the animal, the number and viability of the bacteria were assessed. Serial dilutions of the E. coli stock were plated on BHI agar and cultured overnight at 37° C. The colonies on each plate were counted and the number of colony-forming units per ml was calculated. The body weight measurement of the day of the experiment was used to calculate the E. coli dose and E. coli stock was suspended in isotonic saline (N.P.B.I., Emmer-Compascuum, NL) at the concentration needed for infusion (total dose volume for infusion approximately 10 ml/kg). The E. coli suspension was kept on ice until infusion. Antibiotic was used to synchronize the shock induction in the monkeys. Baytril (Baytril 2.5%, Bayer, DE) was used instead of gentamycin, as the strain proved only marginally susceptible to the latter antibiotic. Individual animals were identified by a number or letter combination tattooed on the chest.

### Experimental Design

| GROUP | EXPERIMENTAL | | | |
|---|---|---|---|---|
| (number/ | TREATMENT | | | |
| letter or other | (independent variable, e.g., | | | |
| identification | placebo-treated control group) | | NUMBER | SEX |
| Animal I | i.v. infusion of a lethal dose of | Live E. coli infusion | N = 1 | F |
| | live E. coli | Blood sampling | | |
| | (10E10 CFU/kg) + antibiotic + | No recovery | | |
| | placebo treated | | | |
| Animal II | i.v. infusion of a lethal dose of | Live E. coli infusion | N = 1 | F |
| | live E. coli | Blood sampling | | |
| | (10E10 CFU/kg) + antibiotic + | No recovery (section) | | |
| | NMPF-4, 6, 46; each 5 mg/kg | | | |
| Animal III | i.v. infusion of a lethal dose of | Live E. coli infusion | N = 1 | F |
| | live E. coli | Blood sampling | | |
| | (10E10 CFU/kg) + antibiotic + | Recovery and survival | | |
| | NMPF-4, 6, 46; each 5 mg/kg | | | |

Anesthesia. All animals were fasted overnight prior to the experiment. On the morning of the experiment, the animals were sedated with ketamine hydrochloride (Tesink, NL) and transported to the surgery. The animal was placed on its side on a temperature-controlled heating pad to support body temperature. Rectal temperature was monitored using a Vet-OX 5700. The animals were intubated orally and were allowed to breathe freely. The animals were kept anesthetized using O₂/N₂O/isofluorane inhalation anesthesia during the E. coli infusion and the seven-hour observation period following E. coli challenge, after which the endotracheal tubes were removed and the animals were euthanized or allowed to recover from anesthesia. The femoral or the cephalic vein was cannulated and used for infusing isotonic saline, live E. coli and antibiotic administration. Insensible fluid loss was compensated for by infusing isotonic saline containing 2.5% glucose (Fresenius, 's Hertogenbosch, NL) at a rate of 3.3 ml/kg/hour.

Preparative actions. During anesthesia, the animals were instrumented for measurement of blood pressure (with an automatic cuff), heart rate and body temperature. Isotonic saline was infused at 3.3 ml/kg/hour to compensate for fluid loss. Femoral vessels were cannulated for infusion of E. coli and antibiotics. Temperature-controlled heating pads were used to support body temperature. The monkeys were continuously monitored during E. coli challenge and for the six-hour period following E. coli administration. After seven hours, two animals (the control animal and one treated with NMPF) were sacrificed to compare the direct effect of the compound at the level of histology. The third animal, treated with NMPF, was allowed to recover from anesthesia and was intensively observed during the first 12 hours after recovery, followed by frequent daily observation. The decision to allow the third animal to recover was made after consulting with the veterinarian.

Induction of septic shock. Before the infusion of E. coli, a one-hour pre-infusion monitoring of heart-rate and blood pressure was performed. All three animals received an i.v. injection of E. coli 086 (k61 serotype; ATCC 33985) at a lethal dose of 10×10⁹ CFU/kg bodyweight. In a dose titration study with this batch performed in 1991, this bacterial dose induced lethal shock within eight hours after the start of the infusion. The infusion period was two hours.

Antibiotics. Baytril was administered intravenously immediately after completion of the two hours E. coli infusion (i.v.; dose 9 mg/kg).

Treatment with NMPF. Thirty minutes post-onset of E. coli infusion, the animals were administered a single intravenous bolus injection of a mixer of NMPF oligopeptides. The oligopeptide mixer contained the following NMPF peptides: LQGV (5 mg/kg), (SEQ ID NO:2) (5 mg/kg) and VLPALP (5 mg/kg). These NMPF peptides were dissolved in 0.9% sodium chloride for injection (N.P.B.I., Emmer Compascuum, NL).

### Results (Preliminary Monkey Results)

An anti-shock effect of the test compound on sepsis in the monkey treated with the oligopeptide mixture, namely the inhibition of the effect of the sepsis in this early seven-hour trajectory of this primate model, was observed. Immunomodulatory effects with these peptides have been observed in vitrolex vivo, such as in T-cell assays, the inhibition of pathological Th1 immune responses, suppression of inflammatory cytokines (MIF), increase in production of anti-inflammatory cytokines (IL-10, TGF-β) and immunomodulatory effects on antigen-presenting cells (APC) like dendritic cells and macrophages.

The following organs were weighed and a bacterial count was performed: kidneys, liver, lungs, lymph nodes, and gross lesions.

Tissues of all organs were preserved in neutral aqueous phosphate buffered 4% solution of formaldehyde. Lymphoid organs were cryopreserved. All tissues will be processed for histopathological examination.

### Further Results Obtained in the Three-Monkey Experiment

Monkey 429 (control). Female monkey (5.66 kg) received an i.v. injection of E. coli 086 (10E10 CFU/kg). In a dose titration study with this batch performed in 1991, this bacterial dose induced lethal shock within eight hours after the start of the infusion. The infusion period was two hours. Baytril was administered intravenously immediately after completion of the two-hour E. coli infusion (i.v.; dose 9 mg/kg). After the E. coli injection, the monkey was observed by the authorized veterinarian without knowing which of the monkeys received NMPF treatment. The clinical observations were as follows: vomiting, undetectable pulse, heart arythmia, abnormalities in ECG: signs of ventricle dilatation/heart decompensation (prolonged QRS complex, extra systoles), decreased blood clotting and forced respiration. In addition, there was big fluctuation in heart rate (30 to 150 beats per minute), collapse of both systolic and diastolic blood pressure (35/20 mmHg) and decrease in blood oxygen concentration (80 to 70%). Seven hours after the start of the E. coli infusion, the monkey began to vomit blood and feces and have convulsions. After final examination, the veterinarian did not give permission to let this monkey awake. At this time point, the control monkey was euthanized. Hereafter, post-mortem examination was conducted and internal organs were examined in situ. A number of internal bleedings were found by the pathologist.

Monkey 459 (NMPF). Female monkey (5.44 kg) received an i.v. injection of E. coli 086 (10E10 CFU/kg). In a dose titration study with this batch performed in 1991, this bacterial dose induced lethal shock within eight hours after the start of the infusion. The infusion period was two hours. Thirty minutes after the initiation of E. coli infusion; NMPF was i.v. injected in a single bolus injection. Baytril was administered intravenously immediately after completion of the two-hour E. coli infusion (i.v.; dose 9 mg/kg). After the E. coli injection, this monkey was also observed by the authorized veterinarian without knowing which of the monkeys received NMPF treatment. The clinical observations were as follows: normal pulse, heart sounds normal, normal ECG, higher heart-rate but otherwise stable (180 beats per minute), no hypotension (75/30 mmHg), normal blood oxygen concentration (95 to 85%), lungs sound normal, normal turgor. Seven hours after the start of the E. coli infusion, the clinical condition of the monkey was stable. After final examination, the veterinarian did give permission to let this monkey awake due to her stable condition. In order to compare the hematological and immunological parameters between the control and NMPF-treated monkey, at this time point NMPF-treated monkey 459 was euthanized. Hereafter, post-mortem examination was conducted and internal organs were examined in situ. No macroscopic internal bleedings were found by the pathologist.

Monkey 427 (NMPF). A female monkey (4.84 kg) received an i.v. injection of E. coli 086 (10E10 CFU/kg). In a dose titration study with this batch performed in 1991, this bacterial dose induced lethal shock within eight hours after the start of the infusion. The infusion period was 2 hours. 30 min. after the initiation of E. coli infusion, NMPF was i.v. injected. Baytril was administered intravenously immediately after completion of the two-hour E. coli infusion (i.v.; dose 9 mg/kg). After the E. coli injection, this monkey was also observed by the authorized veterinarian without knowing which of the monkeys received NMPF treatment. The clinical observations were as follows: normal pulse, heart sounds normal, normal ECG, moderately higher heart-rate but otherwise stable (160 beats per minute), no hypotension (70/30 mmHg), normal blood oxygen concentration (95 to 90%), lungs sound normal, normal turgor. 7 hours after the start of the E. coli infusion, the clinical condition of the monkey was stable. After final examination, the veterinarian gave permission to let the monkey wake up due to her stable condition. Monkey woke up quickly, was alert, and there was a slow disappearance of edema.

### Genomic Experiment

PM1 T-cell line was obtained from American Type Culture Collection (Manassas, Va.) and was cultured at 37° C. in 5% CO₂. These cells were maintained and cultured in RPMI 1640, 10% fetal bovine serum, 2 mM L-glutamine, and antibiotics penicillin and streptomycin. For genomic experiments, cells (2×10⁶/ml) were incubated with phytohemagglutinin (PHA, 10 μg/ml) and IL-2 (200 IU/ml) or PHA, IL-2 and peptide LQGV (10 mg/ml) in a volume of 2 ml in six-well plates. After four hours of cultures, 10×10⁶ cells were washed and prepared for genechip probe arrays experiment. The genechip expression analysis was performed according to the manufacturer's instructions (Expression Analysis, Technical Manual, Affymetrix Genechip). The following major steps outline Genechip Expression Analysis: 1) Target preparation, 2) Target hybridization, 3) Experiment and fluidics station setup, 4) Probe Array washing and staining, 5) Probe array scan and 6) Data analysis.

### Results

### Genomic Experiment

The gene-chip expression analysis revealed that LQGV treatment of PM1 (T-cell line) cells for four hours in the presence of PHA/IL-2 down-regulated at least 120 genes more than two-fold as compared to control PM1 cells (stimulated with PHA/IL-2) only. Moreover, at least six genes were up-regulated more than two-fold in peptide-treated cells as compared to control cells.

Down-Regulated Genes Due to Treatment with LQGV in Genomics Experiment

### Fold Change/Descriptions

21.2 M11507 Human transferrin receptor mRNA, complete cds (_{—}5, _M, _{—}3 represent transcript regions 5 prime, Middle, and 3 prime respectively)

10.1 Human (c-myb) gene, complete primary cds, and five complete alternatively spliced cds (U22376/FEATURE=cds#5/DEFINITION=HSU22376)

9.7 Cluster Incl. X68836:H. sapiens mRNA for S-adenosylmethionine synthetase (cds=(65,1252)/gb=X68836/gi=36326/ug=Hs.77502/len=1283)

9.3 M97935 H. sapiens transcription factor ISGF-3 mRNA, complete cds (_{—}5, _MA, MB, _{—}3 represent transcript regions 5 prime, MiddleA, MiddleB, and 3 prime respectively)

8.7 Human mRNA for phosphatidylinositol transfer protein (PI-TPβ), complete cds (D30037/FEATURE=/DEFINITION=HUMPITPB)

7.5 Cluster Incl. U28964:H. sapiens 14-3-3 protein mRNA, complete cds (cds=(126,863)/gb=U28964/gi=899458/ug=Hs.75103/len=1030)

6.7 Human CDK tyrosine 15-kinase WEE1Hu (Wee1Hu) mRNA, complete cds (U10564/FEATURE=/DEFINITION=HSU10564)

6.7 H. sapiens E2F-related transcription factor (DP-1) mRNA, complete cds (L23959/FEATURE=/DEFINITION=HUMDP1A)

6.5 Cluster Incl. W29030:55c4 H. sapiens cDNA (gb=W29030/gi=1308987/ug=Hs.4963/len=758)

6.1 Cluster Incl. U08997:Human glutamate dehydrogenase gene, complete cds (cds=(0.1676)/gb=U08997/gi=478987/ug=Hs.239377/len=1677)

5.7 M97935 H. sapiens transcription factor ISGF-3 mRNA, complete cds (_{—}5, _MA, MB, _{—}3 represent transcript regions 5 prime, MiddleA, MiddleB, and 3 prime respectively)

5.6 Cluster Incl. Y00638:Human mRNA for leukocyte common antigen (T200) (cds=(86,4000)/gb=Y00638/gi=34280/ug=Hs. 170121/len=4315)

5.3 Ras-Like Protein Tc21

5.3 H. sapiens mRNA for Fas/Apo-1 (clone pCRTM11-Fasdelta(4,7)) (X83492/FEATURE=exons#1-2/DEFINITION=HSFAS47)

4.8 Cluster Incl. AJ002428:H. sapiens VDAC1 pseudogene (cds=(0.853)/gb=AJ002428/gi=3183956/ug=Hs.201553/len=854)

4.7 Ras-Related Protein Rap1b

4.6 Cluster Incl. AL080119:H. sapiens mRNA; cDNA DKFZp564M2423 (from clone DKFZp564M2423) (cds=(85,1248)/gb=AL080119/gi=5262550/ug=Hs.165998/len=2183)

4.5 Cluster Incl. AF047448:H. sapiens TLS-associated protein TASR mRNA, complete cds (cds=(29,580)/gb=AF047448/gi=2961148/ug=Hs.239041/len=620)

4.5 Cluster Incl. D14710:Human mRNA for ATP synthase alpha subunit, complete cds (cds=(63,1724)/gb=D14710/gi=559324/ug=Hs.155101/len=1857)

4.5 Cluster Incl. X59618:H. sapiens RR2 mRNA for small subunit ribonucleotide reductase (cds=(194,1363)/gb=X59618/gi=36154/ug=Hs.75319/len=2475)

4.5 Human mRNA for annexin II, 5 UTR (sequence from the 5 cap to the start codon) (D28364/FEATURE=/DEFINITION=HUMAI23)

4.5 Cluster Incl. AA477898:zu34f08.r1 H. sapiens cDNA, 5 end/clone=IMAGE-739911/clone_end=5 (gb=AA477898/gi=2206532/ug=Hs.239414/len=449)

4.4 Cluster Incl. L19161:Human translation initiation factor eIF-2γ subunit mRNA, complete cds (cds=(0.1418)/gb=L19161/gi=306899/ug=Hs.211539/len=1440gb=AA477898/gi=2206532/ug=Hs.239414/len=449)

4.4 Human serine/threonine-protein kinase PRP4h (PRP4h) mRNA, complete cds (U48736/FEATURE=/DEFINITION=HSU48736)

4.4 Cluster Incl. L43821:H. sapiens enhancer of filamentation (HEF1) mRNA, complete cds (cds=(163,2667)/gb=L43821/gi=1294780/ug=Hs.80261/len=3817)

4.4 Ras-Like Protein Tc21

4.4 Human (c-myb) gene, complete primary cds, and five complete alternatively spliced cds (U22376/FEATURE=cds#3/DEFINITION=HSU22376)

4.3 Cluster Incl. U18271:Human thymopoietin (TMPO) gene (cds=(313,2397)/gb=U18271/gi=2182141/ug=Hs.170225/len=2796)

4.2 Fk506-Binding Protein, Alt. Splice 2

4.2 Human proliferating cell nuclear antigen (PCNA) gene, promoter region (J05614/FEATURE=mRNA/DEFINITION=HUMPCNAPRM)

4.1 Human insulin-stimulated protein kinase 1 (ISPK-1) mRNA, complete cds (U08316/FEATURE=/DEFINITION=HSU08316)

4.1 Cluster Incl. W28732:50h7 H. sapiens cDNA (gb=W28732/gi=1308680/ug=Hs. 177496/len=818)

4.1 Cluster Incl. Y00638:Human mRNA for leukocyte common antigen (T200) (cds=(86,4000)/gb=Y00638/gi=34280/ug=Hs.170121/len=4315)

4 H. sapiens putative purinergic receptor P2Y10 gene, complete cds (AF000545/FEATURE=cds/DEFINITION=HSAF000545)

3.8 Cluster Incl. U08997:Human glutamate dehydrogenase gene, complete cds (cds=(0.1676)/gb=U08997/gi=478987/ug=Hs.239377/len=1677)

3.6 Human mRNA for raf oncogene (X03484/FEATURE=cds/DEFINITION=HSRAFR)

3.6 Cluster Incl. M32886:Human sorcin CP-22 mRNA, complete cds (cds=(12,608)/gb=M32886/gi=338481/ug=Hs. 117816/len=952)

3.6 H. sapiens GTP-binding protein (RAB1) mRNA, complete cds (M28209/FEATURE=/DEFINITION=HUMRAB1A)

3.5 Human FKBP-rapamycin-associated protein (FRAP) mRNA, complete cds (L34075/FEATURE=/DEFINITION=HUMFRAPX)

3.5 Human DNA topoisomerase II (top2) mRNA, complete cds (J04088/FEATURE=/DEFINITION=HUMTOPII)

3.4 Human translation initiation factor eIF-2γ subunit mRNA, complete cds (L19161/FEATURE=/DEFINITION=HUMIEF2G)

3.4 Human mRNA for pre-mRNA splicing factor SRp20, 5 UTR (sequence from the 5 cap to the start codon) (D28423/FEATURE=/DEFINITION=HUMPSF82)

3.4 Cluster Incl. AA442560:zv75g07.r1 H. sapiens cDNA, 5 end/clone=IMAGE-759516/clone_end=5 (gb=AA442560/gi=2154438/ug=Hs.135198/len=566)

3.4 Cluster Incl. X98248:H. sapiens mRNA for sortilin/cds=(21,2522) (gb=X98248/gi=1834494/ug=Hs.104247/len=3723)

3.3 Cluster Incl. AB020670:H. sapiens mRNA for KIAA0863 protein, complete cds (cds=(185,3580)/gb=AB020670/gi=4240214/ug=Hs. 131915/len=4313)

3.3 Cluster Incl. W28869:53h2 H. sapiens cDNA (gb=W28869/gi=1308880/ug=Hs.74637/len=975)

3.3 Cluster Incl. Z12830:H. sapiens mRNA for SSR alpha subunit/cds=(29,889) (gb=Z12830/gi=551637/ug=Hs.76152/len=974)

3.3 Cluster Incl. AL021546:Human DNA sequence from BAC 15E1 on chromosome 12. Contains Cytochrome C Oxidase Polypeptide VIa-liver precursor gene, 60S ribosomal protein L31 pseudogene, pre-mRNA splicing factor SRp30c gene, two putative genes, ESTs, STSs and putative CpG islands (cds=(0.230)/gb=AL021546/gi=2826890/ug=Hs.234768/len=547)

3.2 Cluster Incl. U78082:Human RNA polymerase transcriptional regulation mediator (h-MED6) mRNA, complete cds (cds=(50,523)/gb=U78082/gi=2618737/ug=Hs.167738/len=885)

3.2 H. sapiens RbAp48 mRNA encoding retinoblastoma binding protein (X74262/FEATURE=cds/DEFINITION=HSRBAP48)

3.1 Cluster Incl. M64174:Human protein-tyrosine kinase (JAK1) mRNA, complete cds (cds=(75,3503)/gb=M64174/gi=190734/ug=Hs.50651/len=3541)

3.1 Cluster Incl. AI862521:wj15a06.x1 H. sapiens cDNA, 3 end/clone=IMAGE-2402866/clone_end=3 (gb=AI862521/gi=5526628/ug=Hs.146861/len=606)

3.1 Cluster Incl. W27517:31h6 H. sapiens cDNA (gb=W27517/gi=1307321/ug=Hs. 13662/len=732)

3 Human rab GDI mRNA, complete cds (D13988/FEATURE=/DEFINITION=HUMRABGDI)

3 Cluster Incl. AL080119:H. sapiens mRNA; cDNA DKFZp564M2423 (from clone DKFZp564M2423) (cds=(85,1248)/gb=AL080119/gi=5262550/ug=Hs.165998/len=2183)

3 Human cAMP-dependent protein kinase type I-α subunit (PRKAR1A) mRNA, complete cds (M33336/FEATURE=/DEFINITION=HUMCAMPPK)

3 Cluster Incl. L75847:Human zinc finger protein 45 (ZNF45) mRNA, complete cds (cds=(103,2151)/gb=L75847/gi=1480436/ug=Hs.41728/len=2409)

3 Cluster Incl. M21154:Human S-adenosylmethionine decarboxylase mRNA, complete cds (cds=(248,1252)/gb=M21154/gi=178517/ug=Hs.75744/len=1805)

3 Cluster Incl. AA675900:g02504r H. sapiens cDNA, 5 end/clone=g02504/clone_end=5 (gb=AA675900/gi=2775247/ug=Hs. 119325/len=647)

3 Cluster Incl. M97936:Human transcription factor ISGF-3 mRNA sequence (cds=UNKNOWN/gb=M97936/gi=475254/ug=Hs.21486/len=2607)

2 M33336/DEFINITION=HUMCAMPPK Human cAMP-dependent protein kinase type I-α subunit (PRKAR1A) mRNA, complete cds

2 U16720/FEATURE=mRNA/DEFINITION=HSU16720 Human interleukin 10 (IL10) gene, complete cds

2 M33336 HUMCAMPPK Human cAMP-dependent protein kinase type I-α subunit (PRKAR1A) mRNA

2 U50079/FEATURE=/DEFINITION=HSU50079 Human histone deacetylase HD1 mRNA, complete cds

2 U16720/FEATURE=mRNA/DEFINITION=HSU16720 Human interleukin 10 (IL10) gene, complete cds

2 X87212/FEATURE=cds/DEFINITION=HSCATHCGE H. sapiens mRNA for cathepsin C

2 Cluster Incl. AI740522:wg16b07.x1 H. sapiens cDNA, 3 end/clone=IMAGE-2365237/clone_end=3/gb=AI740522

2 M21154/FEATURE=mRNA/DEFINITION=HUMAMD Human S-adenosylmethionine decarboxylase mRNA, complete cds

2 X00737/FEATURE=cds/DEFINITION=HSPNP Human mRNA for purine nucleoside phosphorylase (PNP; EC 2.4.2.1)

2.1 Cluster Incl. AF034956:H. sapiens RAD51D mRNA, complete cds/cds=(124,993)/gb=AF034956/gi=2920581

2.1 Ras Inhibitor Inf

2.1 Cluster Incl. M27749:Human immunoglobulin-related 14.1 protein mRNA, complete cds/cds=(118,759)/gb=M27749

2.1 Ras-Like Protein Tc4

2.1 X2106/FEATURE=cds/DEFINITION=HSBLEO H. sapiens mRNA for bleomycin hydrolase

2.1 D88674/FEATURE=/DEFINITION=D88674 H. sapiens mRNA for antizyme inhibitor, complete cds

2.1 Cluster Incl. H15872:ym22b12.r1 H. sapiens cDNA, 5 end/clone=IMAGE-48838/clone_end=5/gb=H15872

2.1 Cluster Incl. L07541:Human replication factor C, 38-kDa subunit mRNA, complete cds/cds=(9,1079)/gb=L07541

2.1 V01512/FEATURE=mRNA#1/DEFINITION=HSCFOS Human cellular oncogene c-fos (complete sequence)

2.1 Cluster Incl. L23959:H. sapiens E2F-related transcription factor (DP-1) mRNA, complete cds/cds=(37,1269)

2.1 Stimulatory Gdp/Gtp Exchange Protein For C-Ki-Ras P21 And 5 mg P21

2.1 Cluster Incl. L13943:Human glycerol kinase (GK) mRNA exons 1-4, complete cds/cds=(66,1640)/gb=L13943/gi=348166

2.1 Cluster Incl. X78925:H. sapiens HZF2 mRNA for zinc finger protein/cds=(0.2198)/gb=X78925/gi=498722/ug=Hs.2480

2.1 X74794/FEATURE=cds/DEFINITION=HSP1CDC21 H. sapiens P1-Cdc21 mRNA

2.1 U78733/FEATURE=mRNA#1/DEFINITION=HSSMAD2S8 H. sapiens mad protein homolog Smad2 gene, exon 11

2.2 Cluster Incl. L07540:Human replication factor C, 36-kDa subunit mRNA, complete cds/cds=(9,1031)/gb=L07540

2.2 Cluster Incl. AF040958:H. sapiens lysosomal neuraminidase precursor, mRNA, complete cds/cds=(129,1376)

2.2 D00596/FEATURE=cds/DEFINITION=HUMTS1 H. sapiens gene for thymidylate synthase, exons 1, 2, 3, 4, 5, 6, 7,

2.2 Cluster Incl. AI659108:tu08c09.x1 H. sapiens cDNA, 3 end/clone=IMAGE-2250448/clone_end=3/gb=AI659108

2.2 Cluster Incl. AF042083:H. sapiens BH3 interacting domain death agonist (BID) mRNA, complete cds/cds=(140,727)

2.2 Cluster Incl. W28907:53e12 H. sapiens cDNA/gb=W28907/gi=1308855/ug=Hs. 111429/len=989

2.3 Cluster Incl. AF073362:H. sapiens endo/exonuclease Mre11 (MRE11A) mRNA, complete cds/cds=(0.2126)

2.3 E. coli/REF=J04423/DEF=E. coli bioD gene dethiobiotin synthetase/LEN=676 (−5 and −3 represent transcript

2.3 Cluster Incl. D59253:Human mRNA for NCBP interacting protein 1, complete cds/cds=(36,506)/gb=D59253

2.3 M21154/FEATURE=mRNA/DEFINITION=HUMAMD Human S-adenosylmethionine decarboxylase mRNA, complete cds

2.3 Proto-Oncogene C-Myc, Alt. Splice 3, Orf 114

2.3 Cluster Incl. W26787:15d8 H. sapiens cDNA/gb=W26787/gi=1306078/ug=Hs. 195188/len=768

2.4 L12002/FEATURE=/DEFINITION=HUMITGA4A Human integrin alpha 4 subunit mRNA, complete cds

2.4 Cluster Incl. M55536:Human glucose transporter pseudogene/cds=UNKNOWN/gb=M55536/gi=183299/ug=Hs.121583 2.4 X98743/FEATURE=cds/DEFINITION=HSRNAHELC H. sapiens mRNA for RNA helicase (Myc-regulated dead box protein)

2.4 S75881/FEATURE=/DEFINITION=S75881 A-myb=DNA-binding transactivator {3 region} [human, CCRF-CEM T leukemia line, mRNA Partial, 831 nt]

Cluster Incl. AF050110:H. sapiens TGFb inducible early protein and early growth response protein alpha genes, complete cds/cds=(123,1565)/gb=AF050110/gi=3523144/ug=Hs.82173/len=2899

Cluster Incl. M86667:H. sapiens NAP (nucleosome assembly protein) mRNA, complete cds/cds=(75,1250)/gb=M86667/gi=189066/ug=Hs.179662/len=1560

U17743/FEATURE=/DEFINITION=HSU17743 Human JNK activating kinase (JNKK1) mRNA, complete cds

2.5 Cluster Incl. U90549:Human non-histone chromosomal protein (NHC) mRNA, complete cds/cds=(691,963)/gb=U90549/gi=2062699/ug=Hs.63272/len=1981

Cluster Incl. U31382:Human G protein gamma-4 subunit mRNA, complete cds/cds=(98,325)/gb=U31382/gi=995916/ug=Hs.32976/len=670

Cluster Incl. S81916:phosphoglycerate kinase {alternatively spliced} [human, phosphoglycerate kinase deficient patient with episodes of muscle, mRNA Partial Mutant, 307 nt]/cds=(0.143)/gb=S81916/gi=1470308/ug=Hs.169313/len=307

Cluster Incl. M64595:Human small G protein (Gx) mRNA, 3 end/cds=(0.542)/gb=M64595/gi=183708/ug=Hs.173466/len=757

Serine Hydroxymethyltransferase, Cytosolic, Alt. Splice 3

2.5 U88629/FEATURE=cds/DEFINITION=HSU88629 Human RNA polymerase II elongation factor ELL2, complete cds

2.5 Cluster Incl. U72518:Human destrin-2 pseudogene mRNA, complete cds/cds=(268,798)/gb=U72518/gi=1673523/ug=Hs.199299/len=1057

2.5 Cluster Incl. L14595:Human alanine/serine/cysteine/threonine transporter (ASCT1) mRNA, complete cds

2.5 Cluster Incl. AB014584:H. sapiens mRNA for KIAA0684 protein, partial cds/cds=(0.2711)/gb=AB014584/gi=3327181/ug=Hs.24594/len=4124

2.5 Cluster Incl. AI924594:wn57a11.x1 H. sapiens cDNA, 3 end/clone=IMAGE-2449532/clone_end=3/gb=AI924594/gi=5660558/ug=Hs.122540/len=685

2.5 U68111/FEATURE=mRNA/DEFINITION=HSPPP1R2E6 Human protein phosphatase inhibitor 2 (PPP1R2) gene, exon 6

2.5 Cluster Incl. AL009179:dJ97D16.4 (Histone H₂B)/cds=(25,405)/gb=AL009179/gi=3217024/ug=Hs.137594/len=488

2.6 Cluster Incl. AF091077:H. sapiens clone 558 unknown mRNA, complete sequence/cds=(1,300)/gb=AF091077/gi=3859991/ug=Hs.40368/len=947

2.7 Cluster Incl. M28211:H. sapiens GTP-binding protein (RAB4) mRNA, complete cds/cds=(70,711)/gb=M28211/gi=550067/ug=Hs.234038/len=735

2.6 X69549/FEATURE=cds/DEFINITION=HSRHO2 H. sapiens mRNA for rho GDP-dissociation Inhibitor 2

2.6 Cluster Incl. Z85986:Human DNA sequence from clone 108K11 on chromosome 6p21 Contains SRP20 (SR protein family member), Ndr protein kinase gene similar to yeast suppressor protein SRP40, EST and GSS/cds=(0.932)/gb=Z85986 gi=403-4056/ug=Hs. 152400/len=933

2.6 Zinc Finger Protein, Kruppel-Like

2.7 D10656/FEATURE=/DEFINITION=HUMCRK Human mRNA for CRK-II, complete cds

2.7 M28211/FEATURE=/DEFINITION=HUMRAB4A H. sapiens GTP-binding protein (RAB4) mRNA, complete cds

2.7 Cluster Incl. AB019435:H. sapiens mRNA for putative phospholipase, complete cds/cds=(72,3074)/gb=AB019435/gi=4760646/ug=Hs.125670/len=3088

2.8 U39318/FEATURE=/DEFINITION=HSU39318 Human E2 ubiquitin conjugating enzyme UbCH5C (UBCH5C) mRNA, complete cds

2.9 Cluster Incl. X78711:H. sapiens mRNA for glycerol kinase testis specific 1/cds=(26,1687)/gb=X78711/gi=515028/ug=Hs. 1466/len=1838

2.8 Cluster Incl. W27594:34h4 H. sapiens cDNA/gb=W27594/gi=1307542/ug=Hs.8258/len=702

2.8 X05360/FEATURE=cds/DEFINITION=HSCDC2 Human CDC2 gene involved in cell cycle control

2.8 V00568/FEATURE=cds/DEFINITION=HSMYC1 Human mRNA encoding the c-myc oncogene

2.8 Cluster Incl. L24804:Human (p23) mRNA, complete cds/cds=(232,714)/gb=L24804/gi=438651/ug=Hs.75839/len=782

2.10 Cluster Incl. Y09443:H. sapiens mRNA for alkyl-dihydroxyacetonephosphate synthase precursor/cds=(15,1991)/gb=Y09443/gi=1922284/ug=Hs.22580/len=2074

2.8 Cluster Incl. Z82200:Human DNA sequence from clone 333E23 on chromosome Xq21.1 Contains putative purinergic receptor P2Y10/cds=(0.1019)/gb=Z82200/gi=2370075/ug=Hs. 166137/len=1020

2.9 L05624/FEATURE=/DEFINITION=HUMMKK H. sapiens MAP kinase kinase mRNA, complete cds

2.9 Cluster Incl. D88357:H. sapiens mRNA for CDC2 delta T, complete cds/cds=(27,749)/gb=D88357/gi=3126638/ug=Hs. 184572/len=780

### Up-Regulated Genes Due to LQGV Treatment

### Fold Change/Descriptions

4.9 Cluster Incl. AF043324:H. sapiens N-myristoyltransferase 1 mRNA, complete cds (cds=(10,1500)/gb=AF043324/gi=3005062/ug=Hs.111039/len=4378)

3.3 Cluster Incl. L08096:Human CD27 ligand mRNA, complete cds/cds=(150,731) (gb=L08096/gi=307127/ug=Hs.99899/len=926)

2 Cluster Incl. AF043325:H. sapiens N-myristoyltransferase 2 mRNA, complete cds/cds=(46,1542)/gb=AF043325/gi=3005064/ug=Hs.122647/len=2838

2.1 Cluster Incl. AL031681:dJ862K6.2.2 (splicing factor, arginine/serine-rich 6 (SRP55-2)(isoform 2))/cds=(106,513)

2.1 Cluster Incl. X87838:H. sapiens mRNA for beta-catenin/cds=(214,2559)/gb=X87838/gi=1154853/ug=Hs.171271

2.2 Cluster Incl. AW024285:wt69d06.x1 H. sapiens cDNA, 3 end/clone=IMAGE-2512715/clone_end=3/gb=AW024285

2.2 Cluster Incl. D38524:Human mRNA for 5-nucleotidase/cds=(83,1768)/gb=D38524/gi=633070/ug=Hs.138593

2.2 Cluster Incl. L38935:H. sapiens GT212 mRNA/cds=UNKNOWN/gb=L38935/gi=1008845/ug=Hs.83086/len=1165

2.5 Cluster Incl. L12711:H. sapiens transketolase (tk) mRNA, complete cds/cds=(98,1969)/gb=L12711/gi=388890

2.6 Cluster Incl. AF026029:H. sapiens poly(A) binding protein II (PABP2) gene, complete cds/cds=(1282,2202)

2.8 Cluster Incl. X70683:H. sapiens mRNA for SOX-4 protein/cds=(350,1774)/gb=X70683/gi=36552/ug=Hs.83484

### Further Examples of Use

Examples of different receptor-intracellular signaling pathways involved in different disease pathogenesis where signaling molecules according to the invention find their use include LPS stimulation of antigen-presenting cells (like DC, macrophages, monocytes) through different Toll-like receptors activates different signaling pathways, including MAPK pathways, ERK, JNK and p38 pathways. These pathways directly or indirectly phosphorylate and activate various transcription factors, including Elk-1, c-Jun, c-Fos, ATF-1, ATF-2, SRF, and CREB. In addition, LPS activates the IKK pathway of MyD88, IRAK, and TRAF6. TAK1-TAB2 and MEKK1-ECSIT complexes phosphorylate IKKb, which in turn phosphorylates IκBs. Subsequent degradation of IκBs permits nuclear translocation of NF-κB/Rel complexes, such as p50/p65. Moreover, the PI3K-Akt pathway phosphorylates and activates p65 via an unknown kinase. Some of these pathways could also be regulated by other receptor signaling molecules such as hormones/growth factor receptor tyrosine kinases (PKC/Ras/IRS pathway) and cytokine receptors (JAK/STAT pathway). In the genomic experiment with the T-cell line, several of these genes appeared to be down-regulated or up-regulated by the peptide used (LQGV (SEQ ID NO:1)). It is now clear that other peptides in T-cells and the same and other peptides in other cell types similarly down-regulate or up-regulate several of these transcription factors and signaling molecules. In DC and fertilized egg experiments, NMPF had the ability to modulate growth factor (GM-CSF, VEGF) and LPS signaling. Some diseases associated with dysregulation of NF-κB and related transcription factors are: Atherosclerosis, asthma, arthritis, anthrax, cachexia, cancer, diabetes, euthyroid sick syndrome, AIDS, inflammatory bowel disease, stroke, (sepsis) septic shock, inflammation, neuropathological diseases, autoimmunity, thrombosis, cardiovascular disease, psychological disease, post-surgical depression, wound healing, burn-wounds healing and neurodegenerative disorders.

PKC plays an essential role in T-cell activation via stimulation of, e.g., AP-1 and NF-κB that selectively translocate to the T-cell synapse via the Vav/Rac pathway. PKC is involved in a variety of immunological and non-immunological diseases as is clear from standard text books of internal medicine (examples are metabolic diseases, cancer, angiogenesis, immune-mediated disorders, diabetes etc.).

LPS and ceramide induce differential multimeric receptor complexes, including CD14, CD11b, Fc-gRIII, CD36, TAPA, DAF and TLR4. This signal transduction pathway explains the altered function of monocytes in hypercholesterolemia and lipid disorders.

Oxidized, low-density lipoproteins contribute to stages of the atherogenic process and certain concentrations of oxidized, low-density lipoproteins induce apoptosis in macrophages through signal transduction pathways. These pathways are involved in various vascular diseases such as atherosclerosis, thrombosis, etc.

Bacterial DNA is recognized by cells of the innate immune system. This recognition requires endosomal maturation and leads to activation of NF-κB and the MAPK pathway. Recently, it has been shown that signaling requires the Toll-like receptor 9 (TLR9) and the signaling adaptor protein MyD88. Recognition of dsRNA during viral infection seems to be dependent on intracellular recognition by the dsRNA-dependent protein kinase PKR. TLRs play an essential role in the immune system and they are important in bridging and balancing innate immunity and adaptive immunity. Modulation of these receptors or their downstream signaling pathways is important for the treatment of various immunological conditions such as infections, cancer, immune-mediated diseases, autoimmunity, certain metabolic diseases with immunological component, vascular diseases, inflammatory diseases, etc.

Effect of growth factor PDGF-AA on NF-κB and proinflammatory cytokine expression in rheumatoid synoviocytes: PDGF-AA augmented NF-κB activity and mRNA expression of IL-1b, IL-8 and MIP-1α. Therefore, PDGF-AA may play an important role in progression of inflammation as well as proliferation of synoviocytes in RA.

Dendritic cell (DC) activation is a critical event for the induction of immune responses. DC activation induced by LPS can be separated into two distinct processes: first, maturation, leading to up-regulation of MHC and costimulatory molecules, and second, rescue from immediate apoptosis after withdrawal of growth factors (survival). LPS induces NF-κB transcription factor. Inhibition of NF-κB activation blocked maturation of DCs in terms of up-regulation of MHC and co-stimulatory molecules. In addition, LPS activates the extracellular signal-regulated kinases (ERK), and specific inhibition of MEK1, the kinase which activates ERK, abrogates the ability of LPS to prevent apoptosis but does not inhibit DC maturation or NF-κB nuclear translocation. This shows that ERK and NF-κB regulate different aspects of LPS-induced DC activation. Our DC data and NF-κB data also show the various effects of NMPF peptides on DC maturation and proliferation in the presence or absence of LPS. NMPF peptides modulate these pathways and are novel tools for the regulation of DC function and immunoregulation. This opens new ways for the treatment of immune diseases, particularly those in which the immune system is in disbalance (DC1-DC2, Th1-Th2, regulatory cell, etc.).

DC mediate NK cell activation which can result in tumor growth inhibition. DC and other antigen-presenting cells (e.g., macrophages, B-cells) play an essential role in the immune system and they also help in bridging and balancing innate immunity and adaptive immunity. Modulation of these cells or their downstream signaling pathways is important for the treatment of various immunological conditions such as infections, cancer, immune-mediated diseases, autoimmunity, certain metabolic diseases with immunological component, vascular diseases, inflammatory diseases, etc. There is also evidence in the literature that mast cells play important roles in exerting the innate immunity by releasing inflammatory cytokines and recruitment of neutrophils after recognition of infectious agents through TLRs on mast cells.

In murine macrophages infected with Mycobacterium tuberculosis through the JAK pathway activate STAT1 and activation of STAT1 may be the main transcription factor involved in IFN-g-induced MHC class II inhibition.

Recognition of mannose-binding lectin (MBL) through TLRs influences multiple immune mechanisms in response to infection and is involved in innate immunity. Balance between innate and adoptive immunity is crucial for a balanced immune system and dysregulation in the immune system leads to a different spectrum of diseases such as inflammatory diseases, autoimmunity, infectious diseases, pregnancy-associated diseases (like miscarriage and pre-eclampsia), diabetes, atherosclerosis and other metabolic diseases.

NF-κB is critical for the transcription of multiple genes involved in myocardial ischemia-reperfusion injury. Clinical and experimental studies have shown that myocardial ischemia-reperfusion injury results in activation of the TLRs and the complement system through both the classical and the alternative pathway in myocardial infarction, atherosclerosis, intestinal ischemia, hemorrhagic shock pulmonary injury, and cerebral infarction, etc.

Peroxisome proliferator-activated receptors (PPARs) are ligand-activated transcription factors that function as regulators of lipid and lipoprotein metabolism, glucose homeostasis, influence cellular proliferation, differentiation and apoptosis, and modulation of inflammatory responses. PPARα is highly expressed in liver, muscle, kidney and heart, where it stimulates the beta-oxidative degradation of fatty acids. PPARγ is predominantly expressed in intestine and adipose tissue, where it triggers adipocyte differentiation and promotes lipid storage. Recently, the expression of PPARα and PPARγ was also reported in cells of the vascular wall, such as monocyte/macrophages, endothelial and smooth muscle cells. The hypolipidemic fibrates and the antidiabetic glitazones are synthetic ligands for PPARα and PPARγ, respectively. Furthermore, fatty acid-derivatives and eicosanoids are natural PPAR ligands: PPARα is activated by leukotriene B4, whereas prostaglandin J2 is a PPARγ ligand, as well as of some components of oxidized LDL, such as 9- and 13-HODE. These observations suggested a potential role for PPARs not only in metabolic but also in inflammation control and, by consequence, in related diseases such as atherosclerosis. More recently, PPAR activators were shown to inhibit the activation of inflammatory response genes (e.g., IL-2, IL-6, IL-8, TNF-α and metalloproteases) by negatively interfering with the NF-κB, STAT and AP-1 signaling pathways in cells of the vascular wall. Furthermore, PPARs may also control lipid metabolism in the cells of the atherosclerotic plaque. PPARs are also involved in a variety of immunological and non-immunological diseases as is clear from standard text books of internal medicine (examples are metabolic diseases, cancer, angiogenesis, immune-mediated disorders, diabetes, etc.).

As mentioned above, the nuclear receptor PPARγ is important in adipogenesis and lipid storage and is involved in atherosclerosis. While expressed in adipose tissue, this receptor is also expressed in macrophages and in the colon. In addition, PPARγ is implicated in a number of processes such as cancer and inflammation. Moreover, microbes, via its cognate receptors, typified by the TLRs, possess the capacity to regulate PPARγ-dependent metabolic functions and as such illustrates the intricate interplay between the microbial flora and metabolic control in the alimentary tract.

Cyclo-oxygenase 2 (COX2), an inducible isoform of prostaglandin H synthase, which mediates prostaglandin synthesis during inflammation and which is selectively overexpressed in colon tumors, is thought to play an important role in colon carcinogenesis. Induction of COX2 by inflammatory cytokines or hypoxia-induced oxidative stress can be mediated by nuclear factor κ B (NF-κB). So, inhibition of NF-κB modulates the COX pathway and this inhibition of NF-κB can be therapeutically useful in diseases in which COXs are involved, such as inflammation, pain, cancer (especially colorectal cancer), inflammatory bowel disease and others.

Neuronal subsets in normal brains constitutively express functionally competent C5a receptors. The functional role of C5a receptors revealed that C5a triggered rapid activation of protein kinase C and activation and nuclear translocation of the NF-κB transcription factor. In addition, C5a was found to be mitogenic for undifferentiated human neuroblastoma cells, a novel action for the C5aR. In contrast, C5a protects terminally differentiated human neuroblastoma cells from toxicity mediated by the amyloid A beta peptide. This shows that normal hippocampal neurons as well as undifferentiated and differentiated human neuroblastoma cells express functional C5a receptors. These results show the role of neuronal C5aR receptors in normal neuronal development, neuronal homeostasis, and neuroinflammatory conditions such as Alzheimer's disease.

Activation of the complement system also plays an important role in the pathogenesis of atherosclerosis. The proinflammatory cytokine interleukin (IL)-6 is potentially involved in the progression of the disease. Here, the complement system induces IL-6 release from human vascular smooth-muscle cells (VSMC) by a Gi-dependent pathway involving the generation of oxidative stress and the activation of the redox sensitive transcription factors NF-κB and AP-1. Modulation of the complement system is important for broad ranges of disorders such as blood disorders, infections, some metabolic diseases (diabetes), vascular diseases, transplant rejection and related disorders, autoimmune diseases, and other immunological diseases.

Different transcription factors like NF-κB and intracellular signaling molecules such as different kinases are also involved in multiple drug resistance. So, it is reasonable to believe that NMPF peptides will be effective against multiple drug resistance. Moreover, our genomic data shows that a number of genes and signaling molecules involved in tumorogenesis and metastasis are modulated. In addition, since oligopeptides also have an effect on angiogenesis, these peptides will also be used for the treatment of cancer and related diseases whereby angiogenesis requires modulation.

Proliferative diabetic retinopathy (PDR) is one of the major causes of acquired blindness. The hallmark of PDR is neovascularization (NV), abnormal angiogenesis that may ultimately cause severe vitreous cavity bleeding and/or retinal detachment. Since NMPF peptides have angiogenesis stimulatory as well as inhibitory effects and have the ability to modulate intracellular signaling involved in growth factors (like insulin), pharmacologic therapy with certain NMPF peptides can improve metabolic control (like glucose) or blunt the biochemical consequences of hyperglycemia (through mechanisms such as in which aldose reductase, protein kinase C (PKC), or PPARs are involved). For metabolic control of Type 2 diabetes, NMPF ((SEQ ID NO:1), (SEQ ID NO:3), (SEQ ID NO:29), (SEQ ID NO:33), AQG, LAG, LQA, (SEQ ID NO:2), (SEQ ID NO:22), (SEQ ID NO:173), (SEQ ID NO:31), (SEQ ID NO:41), MTR, (SEQ ID NO:48), LQG, and (SEQ ID NO:175)) are considered. The angiogenesis in PDR could be also treated with the above-mentioned oligopeptides.

Example: Treatment of Trauma-Hemorrhage with Short Oligopeptides

Severe hemorrhage and hemorrhagic shock are common causes of morbidity and mortality in critically ill patients in intensive care. Patients in shock have impaired macro- and microcirculation in various tissue beds. Impaired splanchnic perfusion plays an important role in the development of multiple organ dysfunction owing to enhanced bacterial translocation from the gut and activation of an exacerbated inflammatory cascade. Decreased splanchnic perfusion also leads to the low blood supply to the downstream organs, such as the liver, leading to hepatic dysfunction, which also contributes to multiple organ failure after shock.

### A Fine Balance Between Vasodilators and Vasoconstrictors Maintains Splanchnic Perfusion

Increased systemic production of vasoconstrictors such as epinephrine, angiotensin II, endothelin, and thromboxane A₂ has been observed in experimental models of trauma-hemorrhage and sepsis. These vasoconstrictors not only contribute to the increased total peripheral resistance but also act on the splanchnic vessels and reduce their perfusion rate. The reduced production of vasodilators or the attenuated response of the splanchnic vessel to the vasodilators (endothelial dysfunction) is also observed after severe hemorrhagic or septic shock. Both of these factors contribute to the circulatory disturbance. In addition, these effects induce intestinal hypoxia, reduce nutrient supply, increase production of oxygen free radicals, and increase neutrophil accumulation, leading to damage of the intestinal mucosal barrier and thereby resulting in increased bacterial translocation.

The aim of this study was to determine whether administration of short oligopeptides has any effect on deleterious immune functional parameters after trauma-hemorrhage.

### Materials and Methods

Adult Male-specific pathogen-free Wistar rats (Harlan CPB, Zeist, NL), weighing 350 to 400 g were used after a minimum 7-day acclimation period. The animals were housed under barrier conditions and kept at 25° C. with a 12-hour light/dark cycle. Rats were allowed free access to water and chow (−). All procedures were performed in accordance with the Principles of Laboratory Animal Care (NIH publication No. 86-23, revised 1985) under a protocol approved by the Committee on Animal Research of the Erasmus University (protocol EUR 365).

The rats were fasted overnight but were allowed free access to water before the experiment. Subsequent to endotracheal intubation the rats were mechanically ventilated with an isofluorane (−) N₂O/O₂ mixture at 60 breaths/minute. Body temperature was continuously maintained at 37.5° C. by placing the animals on a thermo controlled “half-pipe” (UNO, NL). Polyethylene tubes (PE-50, Becton Dickinson; St. Michielsgestel, NL) were flushed with heparin and placed via the right carotid artery in the aorta and in the right internal jugular vein. The animals received no heparin before or during the experiment.

Mean arterial pressure (MAP) was measured using transducers (Becton Dickinson) that were connected in line to an electronic recorder (Hewlett Packard, 78354-A, DE) for electronically calculated mean pressures and continuous measurement of the animal's blood pressure. Under semi-sterile conditions a median laparotomy was performed and ultrasonic perivascular flow probes (Transonic Systems Inc, Maastricht, NL) were placed on the common hepatic artery and the portal vein. A supra pubic catheter was placed to monitor the urine production during and after resuscitation.

After an acclimatization period of 20 minutes, the rats were randomized into the following five groups:

Hemorrhagic shock group were bled within 10 minutes to a mean arterial pressure (MAP) of 40 mmHg and maintained at this level for 60 minutes by withdrawing or re-infusing shed blood as needed. Thereafter, the animals were resuscitated with plus/minus four times the volume of the withdrawn blood over 30 minutes with a 0.9% NaCl solution.

The hemorrhagic shock group+peptide A (SEQ ID NO:26) underwent the same procedure as the hemorrhagic shock group but received a single bolus injection of 5 mg/kg peptide A intravenously 30 minutes after the induction of shock.

The hemorrhagic shock group+peptide B (SEQ ID NO:2) underwent the same procedure as the hemorrhagic shock group and received a single bolus injection of 5 mg/kg peptide B intravenously, 30 minutes after the induction of shock.

The hemorrhagic shock group+peptide C (SEQ ID NO:1) underwent the same procedure as the hemorrhagic shock group and received a single bolus injection of 5 mg/kg peptide C intravenously, 30 minutes after the induction of shock.

Sham group underwent the same procedure as the hemorrhagic shock group without performing the hemorrhage or administration of any kind of peptides.

The hepatic arterial blood flow (QHA) and hepatic portal venous blood flow (QVP) were measured with transit time ultrasonic perivascular flow probes, connected to an ultrasonic meter (T201; Transonic Systems Inc, Maastricht). Systemic and hepatic hemodynamics were continuously measured. At regular timepoints arterial blood samples were taken. The animals were killed by withdrawal of arterial blood via the carotid artery.

### Blood, Tissue, and Cell Harvesting Procedure

Plasma collection and storage: Whole arterial blood was obtained at −15, 30, 60, 90, 120, 150 and 180 minutes after induction of shock via the right carotid artery and collected in duplo. 0.2 ml was placed in tubes (Eppendorf EDTA KE/1.3) to be assayed in the coulter counter (−). 0.5 ml was placed in Minicollect tubes (Bio-one, Greiner) centrifuged for 5 minutes, immediately frozen, and stored at −80° C., until assayed. All assays were corrected for the hematocrit.

Measurement of cytokines (still in progress): The levels of IL-6, and IL-10 in the serum were determined by an ELISA (R&D Systems Europe Ltd) according to the manufacturer's instructions.

Histology (still in progress): The alterations in lung, liver, sigmoid and small bowel morphology were examined in sham-operated animals, in animals after trauma-hemorrhage and in animals after trauma-hemorrhage treated with peptide A, B or C. All tissues were collected in duplo. One part was harvested and fixed in formalin (Sigma) and later embedded in paraffin. The other part was placed in tubes (NUNC Cryo Tube™ Vials), quick frozen in liquid nitrogen and stored at −80° C. until assayed.

### Results

Mean Arterial Pressure: MAP dropped in all shock groups significant during the shock phase compared to the control group.

Hematocrit: The hematocrit following trauma-hemorrhage was similar in the different peptide A, B and C treated and non-treated groups. During the shock phase there was a difference of hematocrit in the control group in comparison with the other groups. From the resuscitation phase (90 minutes) there was no significant difference in hematocrit among the control, trauma-hemorrhage, and peptide groups.

Leukocyte Recruitment: During trauma-hemorrhage, leukocytes dropped from 100% at TO in all groups to a minimum of 40.0±11.9%, 42.0±8.7%, 47.3±12.4%, 38.2±7.4% in, respectively, the non-treated, peptide A treated, peptide B treated, and peptide C treated group due to leukocyte accumulation in the splanchnic microcirculation. There was a significant difference in leukocyte concentration between all treated and non-treated trauma-hemorrhage groups, and the control group during the shock phase. No significant difference was noticed between the peptide A, B or C treated animals and the non-treated animals.

Blood Concentrations of Macrophage and Granulocytes: At 180 minutes after the onset of trauma-hemorrhage, concentrations of circulating macrophages (M_{Φ}) and granulocytes were significantly lower in the peptide B and C treated animals compared with the corresponding experimental group. Blood levels of circulating M_{Φ} and granulocytes were 5.556±1.698 10⁹/l in sham-operated animals whereas blood levels were 6.329±1.965 10⁹/l after trauma-hemorrhage, and decreased by 29.9% after administration of peptide B (4.432±0.736 10⁹/l) and 39.2% after administration of peptide C (3.846±0.636 10⁹/l) compared with concentrations after trauma-hemorrhage.

Arterial Hepatic Blood Flow: There was a decrease in the arterial hepatic blood flow in the shock group (18.3±14.3%) and in the peptide A (21.3±9.1%), B (18.1±9.0%) and C (21.2±8.6%) group during the shock period compared with the control group (102.6±23.5%). An increase in blood flow was observed during the reperfusion in the hepatic artery of the shock group (128.9±75.4%) compared with control animals (83.7±24.2%) and the animals treated with peptide B (78.4±28.3%).

Trauma-hemorrhage results in hypoxic stress owing to the absolute reduction in circulating blood volume. In contrast, sepsis is an inflammatory state mainly mediated by bacterial products. It is interesting that these divergent insults reveal similar pathophysiologic alterations in terms of the splanchnic circulation.

Hemorrhagic shock significantly increases leukocyte accumulation in the splanchnic microcirculation owing to the up-regulation of P selectin. The expression of intercellular adhesion molecule within the intestinal muscular vasculature after hemorrhagic shock promotes the local recruitment of leukocytes, and this inflammatory response is accompanied by subsequent impairment of intestinal function.

The adhesion and extravasation of neutrophils not only contribute to the inflammatory response in the splanchnic tissue bed but also induce intestinal microcirculatory failure and dysfunction after severe stress. This is mediated by the induced expression of adhesion molecules, such as selectins and endothelial cell adhesion molecules, on the surface of neutrophils and endothelial cells.

In our shock experiments, leukocyte concentration significantly decreases during hemorrhagic shock compared to the control animals. However, a single dose of peptide B or C administered during resuscitation, decreased concentrations of circulating macrophages and granolocytes 120 min. after the onset of hemorrhagic shock compared to the non-treated animals.

Because some female sex hormones effectively protect the organs from circulatory failure after various adverse circulatory conditions, numerous studies have been performed to clarify the molecular mechanism of, e.g., estradiol action with regard to tissue circulation. Herein, a single dose of peptide was administered following trauma-hemorrhage and various parameters were measured at 3 hours following the induction of sepsis. Treatment with peptides improved or restored immune functional parameters and cardiovascular functions. Therefore, our results show that administration of short oligopeptides (NMPFs) is beneficial in the treatment of critically ill trauma victims experiencing hemorrhagic shock.

### Renal Ischemia-Reperfusion Experiments

Six oligopeptides (i.e., A: (SEQ ID NO:26), B: (SEQ ID NO:2), C: LAG, D: AQG, E: MTR, and F: (SEQ ID NO:42) were tested and compared with PBS (control) in a double blind animal study for each peptide's relative ability to aid recovery in a mouse renal ischemia reperfusion test. In this test, the mice were anesthetized, and one kidney from each mouse was removed. The other kidney was tied off for 25 minutes, and the serum urea levels were allowed to increase. Both before and after tying off, each of the separate peptides was administered to thirty (30) different mice (5 mg oligopeptide/kg body mass intravenously), after which, the mortality of the mice was determined for each oligopeptide as well as was the BUN concentration at two hours, 24 hours and 72 hours. The results are shown in (excluding the results of peptide A (LAGV (SEQ ID NO:26)) obtained in example 1) in Table 7 below.

Under inhalation anesthesia, the left kidney with its artery and vein was isolated and occluded for 25 minutes using a microvascular clamp. During surgery, animals were placed on a heating pad to maintain body temperature at 37° C. Five minutes before placing the clamp, and 5 minutes before releasing the clamp, 5 mg/kg of peptide, dissolved in 0.1 mL of sterile saline, was administered intravenously. After reperfusion of the left kidney the right kidney was removed. Kidney function was assessed by measuring blood urea nitrogen before clamping, and at 2, 24, and 72 hours after reperfusion.

TABLE 7
Results - mortality at 72 hours post-reperfusion

| | A | B | | | | F |
|---|---|---|---|---|---|---|
| | (LAGV) | (AQGV) | | | | (MTRV) |
| | (SEQ ID | (SEQ ID | C | D | E | (SEQ ID |
| PBS | NO: 26) | NO: 2) | (LAG) | (AQG) | (MTR) | NO: 42) |
| 6/10 | 6/10 | 0/10 | 4/10 | 4/10 | 4/10 | 2/10 |
| *P < (vs PBS) | NS | 0.01 | 0.01 | 0.01 | 0.01 | 0.01 |

*2 × 2 Chi-square test. df = 1

Peptide A was the first peptide administered in the renal ischemia reperfusion test. The personnel who performed the experiments went through a learning curve while working with peptide A (LAGV (SEQ ID NO:26)). During administration of the peptide in the inferior caval vein, some animals experienced moderate blood loss from the site of injection, whereas others did not. Inadvertently the animals were returned to the stable without drinking water present in their cages the first night after surgery. Also, by mistake, the animals that were intended to be sacrificed at 72 hours were killed 48 hours after reperfusion. None of these or other problems were encountered during the experiments with peptides B-F.

As can be seen, mice administered the oligopeptides MTRV (SEQ ID NO:42) and especially (SEQ ID NO:2) did much better in terms of both survival (a significant reduction in mortality versus the PBS control group) and reduced BUN concentration than the control group (PBS) or the group administered the other oligopeptides, with more mice surviving and the serum urea levels being much lower than in the other groups. However, the oligopeptides LAG, AQG, and MTR, in this experiment having no reducing effect on BUN concentration, each caused a significant reduction of mortality compared to the PBS control, where MTR did significantly raise BUN levels in the tested mice at 72 hours.

One oligopeptide (A: LAGV (SEQ ID NO:26)) was retested for its capacity to reduce BUN levels in the mice test for the reasons as described above. The results are shown in Table 10 below. As can be seen, mice administered the oligopeptide LAGV (SEQ ID NO:26) now did much better in terms of both survival (a significant reduction in mortality versus the PBS control group) and reduced BUN concentration than the control group (PBS).

Four additional oligopeptides (G (SEQ ID NO:29), H (SEQ ID NO:3), I (SEQ ID NO:1) and J (LQG) were tested for there capacity to reduce BUN levels in the mice test as described above. The results are shown in Table 10 below. As can be seen, mice administered the oligopeptide LQG did show reduced BUN concentration early in the experiment (at 24 hours post-reperfusion) and mice administered VLPALPQ (SEQ ID NO:29) did much better in terms of reduced BUN concentration late in the experiment (at 72 hours post-reperfusion) than the control group (PBS) or the group administered the other oligopeptides, with more mice surviving and the serum urea levels being much lower than in the other groups.

TABLE 8
BUN after 25 minutes renal ischemia tested in mice with peptides A-J

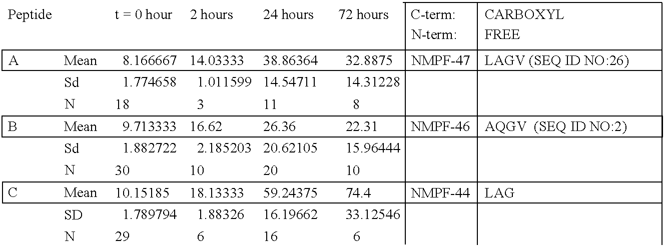

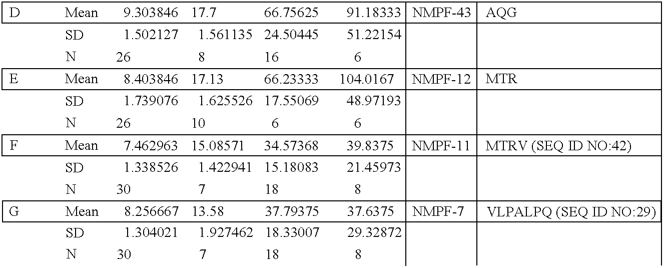

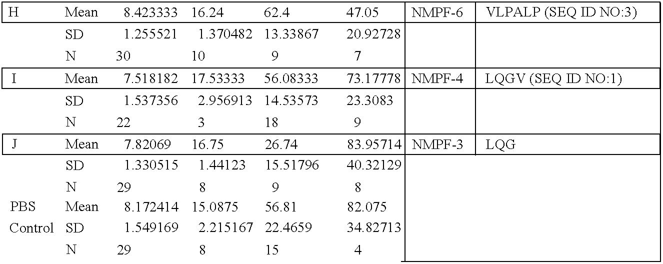

### At 24 Hours Post-Reperfusion Statistical Analyses Revealed P-Values of:

| | | | |
|---|---|---|---|
| A | p = 0.0491 NMPF-47 | LAGV | (SEQ ID NO:26) |
| B | p = 0.0008 NMPF-46 | AQGV | (SEQ ID NO:2) |
| C | p = 0.9248 NMPF-44 | LAG | |
| D | p = 0.4043 NMPF-43 | AQG | |
| E | p = 0.1848 NMPF-12 | MTR | |
| F | p = 0.0106 NMPF-11 | MTRV | (SEQ ID NO:42) |
| G | p = 0.1389 NMPF-7 | VLPALPQ | (SEQ ID NO:29) |
| H | p = 0.5613 NMPF-6 | VLPALP | (SEQ ID NO:3) |
| I | p = 0.9301 NMPF-4 | LQGV | (SEQ ID NO:1) |
| J | p = 0.0030 NMPF-3 | LQG | |

### At 24 Hours Post-Reperfusion Statistical Analyses Revealed P-Values of:

| | | | |
|---|---|---|---|
| A | p = 0.0017 NMPF-47 | LAGV | (SEQ ID NO:26) |
| B | p < 0.0001 NMPF-46 | AQGV | (SEQ ID NO:2) |
| C | p = 0.8186 NMPF-44 | LAG | |
| D | p = 0.2297 NMPF-43 | AQG | |
| E | p = 0.0242 NMPF-12 | MTR | |
| F | p = 0.0021 NMPF-11 | MTRV | (SEQ ID NO:42) |
| G | p = 0.0049 NMPF-7 | VLPALPQ | (SEQ ID NO:29) |
| H | p = 0.3297 NMPF-6 | VLPALP | (SEQ ID NO:3) |
| I | p = 0.8328 NMPF-4 | LQGV | (SEQ ID NO:1) |
| J | p = 0.9445 NMPF-3 | LQG | |

P values were calculated by Mann Whitney U-test (SPSS for Windows).

To determine dose-response relationships, two peptides (D (AQG, having a good effect on mortality on the mice tested in Example 1) and B (AQGV (SEQ ID NO:2)), also having superior effect on BUN of the mice tested in Example 1) were also tested in a dose-response manner in the mice test as described above. Peptides were tested at 0.3, 1, 3, 10 and 30 mg/kg dosages given as described in Example 1. P values (calculated by Mann Whitney U-test (SPSS for Windows)) of serum urea levels of PBS compared to peptide D groups at 72 hours post-clamping were at 0.3 mg/kg 0.001, at 1 mg/kg 0.009, at 3 mg/kg 0.02, at 10 mg/kg 0.000, and at 30 mg/kg 0.23, for peptide B groups these P-values were 0.88, 0.054, 0.000, 0.001 and 0.003. As can be seen, peptide D (AQG) did reduce BUN levels surprisingly well at the lower dosages tested, as compared with peptide B (AQGV (SEQ ID NO:2)), while the beneficial effect on mortality was also still notable at the lower dosages tested.

TABLE 9
Mortality in dose-response experiment

| | 24 hours | 72 hours |
|---|---|---|
| PBS | 0-9 | 4-8 |
| AQG 0.3 mg/kg | 0-10 | 2-8 |
| AQG 1.0 mg/kg | 0-10 | 1-8 |
| AQG 3.0 mg/kg | 0-10 | 0-10 |
| AQG 10.0 mg/kg | 0-8 | 1-10 |
| AQG 30.0 mg/kg | 0-8 | 1-8 |
| AQGV (SEQ ID NO: 2) 0.3 mg/kg | 0-9 | 2-10 |
| AQGV (SEQ ID NO: 2) 1.0 mg/kg | 0-10 | 1-8 |
| AQGV (SEQ ID NO: 2) 3.0 mg/kg | 1-10 | 0-10 |
| AQGV (SEQ ID NO: 2) 10.0 mg/kg | 0-10 | 0-8 |
| AQGV (SEQ ID NO: 2) 30.0 mg/kg | 0-8 | 3-10 |

TABLE 10
Urea Levels in dose-response experiment

| | 24 hours | 72 hours |
|---|---|---|
| PBS | 57.8 | 85.4 |
| Peptide D (AQG) 0.3 mg/kg | 38.4 | 30.4 |
| Peptide D (AQG) 1.0 mg/kg | 48.4 | 38.4 |
| Peptide D (AQG) 3.0 mg/kg | 39.3 | 40.3 |
| Peptide D (AQG) 10.0 mg/kg | 46.8 | 25.8 |
| Peptide D (AQG) 30.0 mg/kg | 52.8 | 58.9 |
| Peptide B (AQGV) (SEQ ID NO: 2) 0.3 mg/kg | 62.4 | 86.7 |
| Peptide B (AQGV) (SEQ ID NO: 2) 1.0 mg/kg | 50.0 | 52.6 |
| Peptide B (AQGV) (SEQ ID NO: 2) 3.0 mg/kg | 37.4 | 19.6 |
| Peptide B (AQGV) (SEQ ID NO: 2) 10.0 mg/kg | 41.2 | 37.1 |
| Peptide B (AQGV) (SEQ ID NO: 2) 30.0 mg/kg | 47.8 | 38.0 |
| standard error | | |
| PBS | 7.1 | 14.7 |
| Peptide D (AQG) 0.3 mg/kg | 8.6 | 3.5 |
| Peptide D (AQG) 1.0 mg/kg | 7.2 | 10.2 |
| Peptide D (AQG) 3.0 mg/kg | 3.5 | 10.7 |
| Peptide D (AQG) 10.0 mg/kg | 8.0 | 3.4 |
| Peptide D (AQG) 30.0 mg/kg | 9.5 | 12.9 |
| Peptide B (AQGV) (SEQ ID NO: 2) 0.3 mg/kg | 10.8 | 14.1 |
| Peptide B (AQGV) (SEQ ID NO: 2) 1.0 mg/kg | 11.7 | 14.3 |
| Peptide B (AQGV) (SEQ ID NO: 2) 3.0 mg/kg | 7.6 | 2.6 |
| Peptide B (AQGV) (SEQ ID NO: 2) 10.0 mg/kg | 8.5 | 6.9 |
| Peptide B (AQGV) (SEQ ID NO: 2) 30.0 mg/kg | 5.8 | 7.8 |

TABLE 11
Statistical significance/p values (Mann Whitney U-Test) of serum
urea levels in dose-response experiment 72 hours post-clamping.
PBS control compared to peptide administered groups. (See, FIG. 3.)

| | 72 hours |
|---|---|
| PBS | NA |
| AQG 0.3 mg/kg | 0.001 |
| AQG 1.0 mg/kg | 0.009 |
| AQG 3.0 mg/kg | 0.02 |
| AQG 10.0 mg/kg | 0.000 |
| AQG 30.0 mg/kg | 0.23 |
| AQGV (SEQ ID NO: 2) 0.3 mg/kg | 0.88 |
| AQGV (SEQ ID NO: 2) 1.0 mg/kg | 0.054 |
| AQGV (SEQ ID NO: 2) 3.0 mg/kg | 0.000 |
| AQGV (SEQ ID NO: 2) 10.0 mg/kg | 0.001 |
| AQGV (SEQ ED NO: 2) 30.0 mg/kg | 0.003 |

What is claimed is:

1. A method for treating an ischemia-reperfusion injury, said method comprising:
diagnosing an ischemia-reperfusion injury in a subject, and
administering to the subject a composition comprising an oligopeptide, or pharmaceutically acceptable salt or ester thereof, said oligopeptide, salt or ester constituting a means for reducing production of NO by a cell, together with a pharmaceutically acceptable excipient.

2. A method for treating an ischemia-reperfusion injury, said method comprising:
diagnosing an ischemia-reperfusion injury in a subject, and
administering to the subject a composition comprising an oligopeptide, or pharmaceutically acceptable salt or ester thereof, said oligopeptide salt or ester constituting a means for modulating translocation and/or activity of a gene transcription factor present in a cell, together with a pharmaceutically acceptable excipient.

3. The method according to claim 1, wherein said means for reducing production of NO by a cell additionally modulates translocation and/or activity of a gene transcription factor present in the cell.

4. The method according to claim 2, wherein said gene transcription factor comprises an NF-κB/Rel protein.

5. The method according to claim 3, wherein said modulating translocation and/or activity of a gene transcription factor allows modulation of TNF-α production by the cell.

6. The method according to claim 5, wherein said TNF-α production is reduced.

7. The method according to claim 1, wherein said ischemia-reperfusion injury comprises acute ischemia-reperfusion injury.

8. The method according to claim 7, wherein said ischemia-reperfusion injury comprises cerebro-ischemia-reperfusion injury, renal-ischemia-reperfusion injury, and/or cardiovascular ischemia-reperfusion injury.

9. The method according to claim 1, wherein said method further comprises:
administering to the subject, in said composition, means for reducing production of NO by a cell.

10. The method according to claim 9, wherein said means for reducing production of NO by a cell comprises at least two oligopeptides able to reduce NO production by a cell.

11. The method according to claim 10, wherein said at least two oligopeptides are selected from the group LQGV (SEQ ID NO:1), AQGV (SEQ ID NO:2), and VLPALP (SEQ ID NO:3).

12. A medicament comprising:
an oligopeptide constituting a means for reducing production of NO in a cell, said oligopeptide present in an amount sufficient to reduce NO production in a cell when administered to a cell, said oligopeptide admixed with a pharmaceutically acceptable excipient, together with printed instructions directing treatment of a subject resulting in NO production in a cell.

13. The composition of claim 13, comprising at least two oligopeptides able to reduce NO production by a cell.

14. The method according to claim 2, wherein said ischemia-reperfusion injury comprises cerebro-ischemia-reperfusion injury, renal-ischemia-reperfusion injury, and/or cardiovascular ischemia-reperfusion injury.

15. The method according to claim 2, wherein said method further comprises:
reducing NO production by a cell of the subject.

16. The method according to claim 3, wherein said gene transcription factor comprises an NF-κB/Rel protein.

17. A method of treating ischemia-reperfusion injury in a subject by reducing NO production by the subject's macrophages, the method comprising:
administering to the subject a composition comprising an oligopeptide, or pharmaceutically acceptable salt or ester thereof, constituting a means for reducing production of NO by a cell, and a pharmaceutically acceptable excipient.

18. The method according to claim 1, wherein the oligopeptide is selected from the group consisting of LQGV (SEQ BD NO:1), AQGV (SEQ ID NO:2), MTRV (SEQ ID NO:42), QVVC (SEQ ID NO:43), and combinations of any thereof.

19. The method according to claim 2, wherein the oligopeptide is selected from the group consisting of LQGV (SEQ ID NO:1), AQGV (SEQ ID NO:2), MTRV (SEQ ID NO:42), QVVC (SEQ ID NO:43), and combinations of any thereof.

20. The method according to claim 19, wherein the oligopeptide is selected from the group consisting of LQGV (SEQ BD NO:1), AQGV (SEQ BD NO:2), MTRV (SEQ ID NO:42), QVVC (SEQ ID NO:43), and combinations of any thereof.
